(19) United States
(12) Patent Application Publication
(10) Pub. No.: US 2024/0156391 A1
(43) Pub. Date: May 16, 2024

(54) NOVEL NEURAL CONTROL SIGNALS FOR THERAPEUTIC BEHAVIORAL MODULATION IN EATING-RELATED DISORDERS

(71) Applicant: The Board of Trustees of the Leland Stanford Junior University, Stanford, CA (US)

(72) Inventors: Daniel Alves Neiva Barbosa, Stanford, CA (US); Vivek Buch, Stanford, CA (US); Sandra Gattas, Stanford, CA (US); Casey Halpern, Stanford, CA (US); Yuhao Huang, Stanford, CA (US); Rajat Shivacharan, Stanford, CA (US)

(21) Appl. No.: 18/552,570

(22) PCT Filed: Apr. 1, 2022
(86) PCT No.: PCT/US22/23129

§ 371 Date: Sep. 26, 2023

Related U.S. Application Data
(60) Provisional application No. 63/220,432, filed on Jul. 9, 2021.
(60) Provisional application No. 63/170,404, filed on Apr. 2, 2021.

(51) Int. Cl. A61B 5/374; A61B 5/00

(52) U.S. Cl. CPC A61B 5/374; A61B 5/4836

(57) ABSTRACT

Provided herein, inter alia, are methods for detecting brain structure modulations associated with impaired inhibitory control disorders (ICD). The methods are useful for treating of a variety of neurological and psychiatric conditions, and can include neurostimulation to specific regions of the brain.

### RELATED APPLICATIONS

This application claims the benefit of priority under 35 U.S.C. § 119(e) to U.S. Provisional Application No. 63/170,404, filed Apr. 2, 2021, and U.S. Provisional Application No. 63/220,432, filed Jul. 9, 2021, the entire contents of each of which is incorporated herein by reference in their entireties.

### STATEMENT AS TO RIGHTS TO INVENTIONS MADE UNDER FEDERALLY SPONSORED RESEARCH AND DEVELOPMENT

This invention was made with government support under contract NS103446 awarded by the National Institutes of Health. The government has certain rights in the invention.

### BACKGROUND

Over the past several decades obesity has increased in prevalence worldwide to epidemic proportions. In the U.S. alone 80 million adults are categorized as obese (BMI >30 kg/m). Along with this alarming rise in obesity, the U.S. has experienced a concomitant rise in obesity-related comorbidities, including hypertension, dyslipidemia, type-2 diabetes, atherosclerotic heart disease, obstructive sleep apnea, and several malignancies, all adversely affecting quality of life and decreasing overall lifespan. Obesity is a major public health challenge costing the US $150 billion annually with obese patients experiencing 46% higher inpatient costs, 27% more physician visits and outpatient costs, and 80% higher spending on prescription drugs than normal-weight individuals.

Despite improved preventative care and treatment efforts, the obesity epidemic continues. Contributing to the epidemic are lack of effective therapies for many persons with LOC eating and obesity, the lack of fundamental understanding of human reward circuitry, that no physiological biomarker for disordered eating has as yet been identified, and that stimulation approaches to treatment of this disorder are not yet successful and, if successful, optimal approaches to responsive stimulation are not known.

Further, current treatments for LOC eating in obese patients are not effective for all. Although bariatric surgery effectively induces weight loss and may ameliorate eating disorder comorbidities of obesity including sensations of LOC, these data are inconsistent-indeed a recent study of bariatric outcomes revealed compromised weight loss following bypass in patients with LOC eating. Moreover, novel cases of LOC eating have been reported to occur after bariatric surgery. One study examining postoperative outcomes in obese patients with pathological eating habits found that short-term improvements in eating behaviors after gastric bypass surgery could erode after 2 years and were associated with weight regain. Additionally, there are serious risks to gastric bypass, including anastomotic leakage, vitamin deficiencies, and death. Many remain obese with persisting comorbidities or regain weight due to LOC eating.

Current treatments for individuals with suboptimal outcomes after bariatric surgery are limited. Also, many patients with obesity refuse to undergo gastric bypass surgery due to the requirement for large gut reconstruction. The surgery is morbid with up to 100% of patients having nutritional deficiencies at long-term follow-up, and an overall surgical complication rate of about 33%. Without surgery, patients are offered CBT and weight loss management options, such as life style interventions, but these are often unsuccessful with typical losses of only about 10% of initial body weight, and continued significant impairment in quality of life.

Several therapies may reduce LOC eating, such as cognitive-behavioral therapy, but lack long-term efficacy. Pharmacological options include antidepressant (i.e., SSRIs, bupropion), anti-seizure (i.e., topiramate), anti-ADHD (i.e., lisdexamfetamine dimesylate), and anti-obesity drugs (i.e., orlistat, liraglutide). These agents can modestly reduce LOC eating, but with return to baseline when discontinued. Antidepressants can complement behavioral therapy and reduce the frequency of food binges, but fail to induce substantial weight-loss and are limited in long-term efficacy. The anti-obesity agent sibutramine hydrochloride demonstrated some efficacy in clinical trials but was removed from the market due to safety concerns. Antiepileptic medications used for pathological eating habits are associated with high rates of discontinuation due to side effects. While new pharmacotherapies are being developed, the long history of polypharmacy failures likely precludes dramatic amelioration of these eating disorders in obesity.

Some of the deficiencies in current treatments are due to the lack of awareness that the cravings and compulsive behaviors common to substance-use disorders and addiction are also seen in obesity, and that these behaviors may be as significant a problem as energy balance and food intake. Thus, examining other addiction-related constructs as they relate to obesity and other food-related conditions seems relevant. Indeed, patients with LOC eating unequivocally exhibit obsessive thoughts and compulsions around food and these sensations may be the most detrimental to obese patients' quality of life. It is possible that a therapeutic intervention that addresses LOC overeating would also be relevant to other addictive behaviors.

Disclosed herein, inter alia, are solutions to these and other problems in the art.

### BRIEF SUMMARY OF THE INVENTION

In an aspect is provided a method of detecting a low frequency modulation in the insular cortex and the hippocampus of a subject, wherein the subject is diagnosed with, or suspected of having, an impaired inhibitory control disorder (ICD), the method including: inserting at least one electrode into each the insular cortex and the hippocampus of the subject; and recording brain wave activity in the insular cortex and the hippocampus of the subject.

In an aspect is provided a method of detecting a low frequency modulation in the insular cortex and the hippocampus of a subject, wherein said subject is diagnosed with, or suspected of having, an impaired inhibitory control disorder (ICD), the method comprising: inserting at least one electrode into each the insular cortex and the hippocampus of the subject; and recording brain wave activity in the insular cortex and the hippocampus of the subject.

In some embodiments, the low frequency modulation comprises a modulation having a frequency between about 0 hertz-8 hertz. In some embodiments, the low frequency modulation comprises a modulation having a frequency between about 3.5 to 7.5 Hz. In some embodiments, low frequency modulation comprises a modulation having a frequency between about 0.1-4 Hz. In some embodiments, the low frequency modulation is an increase of a frequency between about 0 hertz-8 hertz relative to a standard control.

In some embodiments, the ICD comprises a disorder that is associated with a lack of impulse control. In some embodiments, the ICD includes one or more of binge eating, substance abuse, sex addiction or compulsive sexuality, kleptomania, pyromania, trichotillomania, panic disorder, Intermittent Explosive Disorder, compulsive behaviors including gambling, night eating, loss of control eating, emotional or stress eating, compulsive eating, purge behaviors, or suicidal ideation/attempt. In some embodiments, the ICD comprises a disorder that is associated with a lack of impulse control, and includes one or more of binge eating, substance abuse, sex addiction or compulsive sexuality, kleptomania, pyromania, trichotillomania, panic disorder, Intermittent Explosive Disorder, compulsive behaviors including gambling, night eating, loss of control eating, emotional or stress eating, compulsive eating, purge behaviors, or suicidal ideation/attempt.

In some embodiments, the method described herein further comprises administering, in response to the low frequency modulation, a stimulation to the insular cortex and the hippocampus of the subject. In some embodiments, the stimulation is transcranial direct current stimulation (TDCS), transcranial magnetic stimulation (TMS), or low intensity ultrasound stimulation.

### BRIEF DESCRIPTION OF THE DRAWINGS

FIG. 1. Example of an LFP display on the PDMS (patient data repository) from an epilepsy patient. The lower panel is taken from the upper panel with an expanded time scale. An epileptiform discharge is detected (B2 on Channel 4) and treated with a responsive stimulation (Tr). A second detection occurs about 4 seconds later (Channel 4) and responsive stimulation is delivered again.

FIGS. 2A-2F. Mice exhibited increased intake until stabilizing (FIG. 2A). Deep brain stimulation (DBS)-like stimulation administered only acutely blocked intake on 2 alternate days (FIG. 2B). Nucleus accumbens (NAc) DBS suppressed daily food intake when delivered chronically, but there was loss of significance after Day 2 (FIG. 2C). NAc stimulation did not induce a place preference, suggesting the effects were not reinforcing (FIG. 2D). Mice provided with a 1 h of exposure to high fat exhibit episodic binge behavior (FIG. 2E). Manually triggering stimulation at these binge onsets under synchronized video surveillance blocks intake with <10% less stim-on time. HF, high-fat; DBS, deep brain stimulation; *p<0.05 (FIG. 2F).

FIGS. 3A-3F. Continuous time-frequency spectrogram indicating increased poser in low frequency oscillations prior to binge onset (dashed white line) using an off-the-shelf system (FIG. 3A). The corresponding power spectrogram (top) and LFPs (bottom) recorded by integrated RNS System revealing similar changes during binge period (bottom, the box area) (FIG. 3B). This change delta band specifically was detected by a predefined biomarker detector, indicated by marker “A2” and was used for RNS (as shown in FIG. 4 below) (FIG. 3B). These spectrograms and LFPs represent data recorded immediately before and during chow intake (the dotted white line in FIG. 3C and the box area in FIG. 3D) and social interaction (the dotted white line in FIG. 3E and the box area in FIG. 3F), none of which revealed such a robust delta power change and minimal RNS detections and stimulations occurred.

FIGS. 4A-4C. Scheduled stim (10 s on, 20 s off) blocked intake somewhat but this blockade was only significant when stim was delivered manually or in response to delta power change (RNS). Threshold in delta at 20% increase was the power change (RNS) (FIG. 4A). Threshold in delta at 20% increase was the power change biomarker (FIG. 4B). Social interaction with a juvenile was not altered using this biomarker (FIG. 4C). Sch, scheduled stimulation; RNS, responsive stimulation, RND, random *p<0.05.

FIGS. 5A-5C. Gain ($5.00) contrast with significant NAc BOLD signal (p<0.005) (FIG. 5A). Continuous time-frequency spectrograms show increased power (proportional to gain magnitude) in delta during same anticipation phase as fMRI (FIG. 5B). LFPs were run through a benchtop RNS to detect delta power increases. The traces in the solid boxes represent detection. Adjusting parameters resulted in earlier detections (the traces in the dotted boxes) (FIG. 5C).

FIGS. 6A-6C. Components of the RNS System. 3D schematic of neurostimulator and NAc depths (FIG. 6A). Depth contacts superimposed and to-scale on the NAc derived from the Schaltenbran and Wahren Atlas (FIG. 6B). Close-up with dimensions. Cd, caudate, Put, putamen, NAc, nucleus accumbens, ALIC, anterior limb of the internal capsule (FIG. 6C).

FIGS. 7A-7E. Dissecting the human hippocampal-LH feeding circuit with neuroimaging. Tractography on high-resolution data from 178 HCP subjects revealed LH streamlines converge in the dlHPC (“hot spot”) within the hippocampal area (FIG. 7A). Significantly higher normalized streamline counts were observed between the LH and the left (p<0.0001) and right (p<0.0001) dlHPC as compared to the whole hippocampal areas in a clinical dataset (FIG. 7B). rsFC between the dlHPC and LH was significantly different between groups (H=8.3919, df=2, p=0.0151), with a decrease in obese binge eaters (n=10) compared to lean controls (n=14) (p=0.009, adjusted for false discovery rate (FDR) of multiple comparisons) but not lean binge eaters (n=15). Each group had 2 outliers identified and removed in this analysis (FIG. 7C). Tractography-CI between the left dlHPC and the LH was significantly different between groups (F=3.418, p=0.043), with a decrease in obese binger (n=11) compared to lean controls (n=14) (Tukey's post hoc test, p=0.034) but not lean binge eaters (n=15). One outlier was identified and removed from the group of obese binge eaters and two outliers were identified and removed for each of the two other groups in this analysis (FIG. 7D). No differences in tractography-CI between the right dlHPC and LH were found between groups. No outlier was identified among the obese binge eaters in this analysis, while 1 outlier was identified and removed from each other group (FIG. 7E).

FIGS. 8A-8D. Elucidating the Tractography-defined dlHPC. The subsection corresponding to the rostral dentate gyrus (DGR) and the hippocampal proper (rostral CA1 and CA2 subregions) according to the Allen Reference Brain Atlas (see the World Wide Web site at atlas.brain-map.org) was used in the iDISCO brain-clearing procedure (FIG. 8A). The iDISCO cleared sample (indicated by the magnifying glass) was overlaid to the group average hotspot based on streamline probability between the hippocampal area (seed) and lateral hypothalamus (FIG. 8B). Staining for melanin-concentrating hormone containing (MCH+) (Phoenix Pharmaceuticals, Inc) and Alexa Fluor 647 (Thermo Fisher) appear in red and the autofluorescence (channel 480) is depicted in green with the hotspot of high streamline probability to the LH (FIG. 8C). MCH+ projections within hippocampal hotspot in detail (FIG. 8D). Image acquired at light sheet microscope (UltraMicroscope II, LaVision BioTec, Germany). Scale bar=100 um.

FIGS. 9A-9D. Differential hippocampal slow-frequency (˜3-8 Hz) power recruitment during anticipation of highly palatable compared to water-based solution. Group hippocampal coverage (FIG. 9A). Difference spectrograms (palatable-neutral item) in the hotspot (left) and non-hotspot (right) hippocampal contacts are shown in FIG. 9B. The right-side bar indicates mean z-score power difference across channels between the two conditions compared to a null distribution. Outlined clusters reflect significant contiguous time-frequency voxels (p<0.05, cluster-based permutation testing, 1000 permutations) before correction for multiple comparisons. Insets display significant time-frequency clusters after correction for multiple comparisons (FIG. 9B). Quantified dlHPC hotspot slow frequency 3-8 Hz power on a group level (p=0.002, paired non-parametric permutation testing, using channels as observations) (FIG. 9C). 3-8 Hz and 5-8 Hz power time traces in the dlHPC (left) and vmHPC (right) hippocampal sites, respectively (FIG. 9D).

FIG. 10A. Milkshake paradigm design.

FIG. 10B. Topography of exemplary electrodes recordings anticipation of milkshake, tractography-defined hotspot of lateral hypothalamic connections in the hippocampal area and sample used in the iDISCO protocol.

FIGS. 11A-11E. The milkshake paradigm evokes responses in the human frontal opercular and insular cortices. The task paradigm. All subjects performed a food task which involved responding to cues of either palatable (chocolate milkshake) or taste-neutral (water-based solution) liquid and subsequently receiving 3 mL bolus. The anticipatory phase of the task was defined as the onset of the cue to immediately prior to solution delivery (0 to 3 s). The receipt phase of the task was defined from solution delivery to immediately prior to swallowing (3 s to 6 s) (FIG. 11A). Freesurfer parcellation of the insular/opercular cortex. The insula was divided into anterior-posterior subregions based on the central sulcus of the insula. The anterior insula includes the short gyri, the anterior circular sulcus, and the posterior circular sulcus; the posterior insula includes the long gyrus and the inferior circular sulcus. The frontal operculum is defined as the inferior frontal gyrus (FIG. 11B). Magnetic resonance image reconstruction of the insular cortex and the frontal operculum overlaid with translucent cortical surface. All insular/opercular recording contacts are plotted as 3D spheres, color-coded according to subjects, in the Montreal Neurological Institute (MNI) space (FIG. 11C). Time-frequency spectrogram and high-frequency broadband (HFB) activity waveform from an insular electrode in a left long gyrus (left-side circled electrode in FIG. 11C) showing differential responses to palatable vs. taste-neutral cue during anticipation. Time points significantly different between palatable and neutral conditions (p<0.05, cluster-based permutation testing, alpha <0.05) are marked in red (indicated by ovals) along the horizontal axis (FIG. 11D). Effect size was calculated using time periods that were considered significant as marked by red. The vertical dashed lines denote the onset of the solution cue (t=0 s) and solution delivery (t=3 s), respectively. Inset: Distribution of mean HFB activity stratified by palatable and neutral trials (FIG. 11D). Identical data representation as in FIG. 11D is shown in FIG. 11E for an electrode in the right superior circular sulcus of the insula (black circled electrode in FIG. 11B showing differential responses to palatable versus taste-neutral solution during receipt (FIG. 11E).

FIGS. 12A-12D. Topographic differences in insular and frontal opercular neural activity when anticipating and receiving task solutions. Site-by-site differences in anticipatory neural activity. Effect size between the anticipatory responses to palatable versus neutral solution is shown per electrode. Shades of red (the left section of the bar with readings sliding from approximately 0.6 to 1.0, cue-specific) indicate significantly greater response in palatable trials whereas shades of blue (the right section of the bar with readings sliding from approximately −0.6 and −1.0, cue-specific) indicate significantly greater response in taste-neutral trials (FIG. 12A). Green (the middle section of the bar with readings sliding from approximately 0.6 to −0.6, cue-responsive) indicates a significant increase in activity from pre-cue baseline activity, but no difference between palatable or neutral conditions (cue-responsive but not cue-specific) (FIG. 12A). Grey (the sections in the left or the right 3-D models in FIG. 12A having the lighter shading) indicates activity was not different from pre-cue baseline activity (inactive) (FIG. 12A). Proportion of inactive (the left bar for each location), responsive but not discriminative (the middle bar for each location), and discriminative (the right bar for each location) electrodes during anticipation stratified by anatomical location and sidedness (FIG. 12B). Same set of plots as in FIGS. 12A and 12B showing site-by-site differences in receipt neural activity (FIGS. 12C and 12D).

FIGS. 13A-13C. Insular HFB activity is sufficient to classify between anticipation for palatable and taste-neutral solutions on a single trial basis. Parallel coordinates plot displaying normalized HFB power for each observation (n=250 taste-neutral and n=250 palatable trials) as a function of the four features used (4 anticipation time epochs: 0-0.5 and 0.5-1 sec of cue presentation, and 1-2 and 2-3 sec of post-cue fixation). Note that at the second feature, a cluster of taste neutral anticipation trials is observed (FIG. 13A). FIG. 13B shows inter (“group”) and intra-individual Receiver Operating Curves (ROCs) for positive class 1 (neutral anticipation). Inter-individual mean TPR and FPR and AUC were 64%, 36% and 69%, respectively. Intra-individual mean TPR and FPR, and AUC for the three subjects were (4) 69%, 31%, and 74% (7) 64%, 36%, and 69% and (8) 57%, 43%, and 58% respectively (FIG. 13B). Results of statistical testing on classifier performance are shown in FIG. 13C (TPR in blue, FPR in red). First set of values represent group classification performance on observed data: 64% mean TPR and 36% mean FPR across the two classes. Same performance measures were computed following permutations (n=100) of values for a given feature across the two classes. Shuffled features 1, 2, 3, and 4 values yielded the following mean TPR and FPR values: (1) 58.84%, 41.06, (2) 52.56, 47.46, (3) 58.43, 41.46, and (4) 61.94, 38.06. Error bars represent S.E.M. across performance measures on shuffled data (n=100). Observed TPR and FPR significantly differed from features 1, 2, and 3 shuffled data (p<0.01, p<0.01, and p=0.01, respectively). Feature 4 shuffled data did not significantly affect classification performance (p=0.08). Performance using feature 2 shuffled data was significantly diminished compared to performance on shuffled data for any other feature (p=0.02 for features 2 vs. 1, 3, and 4, p_{FDR}=0.002) (FIG. 13C). *p<0.05, **p<0.01

FIGS. 14A and 14B. Comparison of HFB response onset by anatomical location during task anticipation. Histogram showing distribution of response onset latency (ROL) of HFB at the frontal operculum, the anterior insula, the posterior insula and visual regions. Each observation represents the ROL of HFB at the single trial level calculated for all trials across all subjects (palatable and taste-neutral conditions). The vertical line denotes the median ROL time (FIG. 14A). Schematic representing the sequence of HFB activity onset: an early visual response to cue with subsequent delay in activation of the frontal operculum, followed by involvement of anterior insula and subsequently posterior insula (FIG. 14B). The traces in FIG. 14B represent, from left to right, visual, frontal operculum, anterior insula, and posterior insula, respectively.

FIGS. 15A and 15B. Time-locked HFB activity during natural eating stratified by type of food consumed. Simultaneous HFB activity trace and video capture during meal consumption. For each subject, HFB activity from an exemplar electrode in the left posterior insula is shown. Dotted vertical lines denote the moment food was about to enter the subject's mouth, with accompanying images showing the time point that was used for time-locking (FIG. 15A). Mean time-locked HFB waveforms stratified by type of food consumed. Dotted vertical lines denote the immediate time point as food was about to enter the subject's mouth. Time points significantly different between entree versus non-entree near the time of food entering the mouth (p<0.05, cluster-based permutation testing, alpha <0.05) are marked by lines on top of the two traces (“fruit” and “entree”) and along the horizontal axis (FIG. 15B).

FIGS. 16A and 16B. Food-specific responses during anticipation are maintained in task-based and natural eating settings. Topography of response types in the insular/opercular cortex during task-based and natural eating. During eating of a standard meal, a food-specific site (blue electrodes, such as those indicated by solid arrows in the left panel in FIG. 16A) was defined as a significant difference in HFB time-locked activity between food types near the time that food was about to enter a subject's mouth, whereas grey electrodes indicate non-responsive response. During a task, a cue-specific response (red electrodes, such as those indicated by solid arrows in the middle panel in FIG. 16A) was defined as a significant difference between palatable and neutral conditions for either anticipation or receipt of these solutions. Green electrodes (cue-responsive sites, such as those indicated by dotted arrows in the middle panel in FIG. 16A) denote significant HFB activity compared to pre-cue baseline activity, but no difference between palatable and taste-neutral conditions. Grey electrodes (such as those indicated by ovals in the left, the middle, and the right panels in FIG. 16A) denote activity was not different from pre-cue baseline activity. Electrodes visualized are from the three subjects with left posterior insular coverage as shown in FIG. 14 (FIG. 16A). The probability of a channel being food-specific during eating with and without significant response during task anticipation. Left panel of FIG. 16B: Cue-responsive contacts without discriminatory activity during anticipation was not associated with food-specific eating response. However, the presence of cue-specific activity was significantly associated with the presence of food-specific eating response. Right panel of FIG. 16B: Amongst all electrodes, neither receipt-responsive nor receipt-specific responses were associated with food-specific channels.

FIGS. 17A and 17B. Time-frequency spectrogram for mean insulo-opercular activity and effect size quantification by conventional frequency ranges. Time-frequency spectrogram computed separately for palatable and taste-neutral conditions using all insulo-opercular channels (N=168). The vertical dashed lines denote the onset of the solution cue (t=0 s) and solution delivery (t=3 s) (FIG. 17A). Effect size (Cohen's d) calculated between palatable and taste-neutral conditions across all insulo-opercular channels (N=168) were averaged per conventional frequency bands: delta (1-4 Hz), theta (4-8 Hz), alpha (8-12 Hz), beta(15-25 Hz), gamma bands (25-50 Hz) and HFB (70-170 Hz). The first second after stimulus onset was used. Significantly higher effect size (favoring palatable conditions) were found in gamma and HFB power after cue presentation and solution delivery. Theta power during anticipation was also associated with significantly higher effect size favoring the palatable condition (FIG. 17B).

FIGS. 18A-18C. Site-by-site differential insulo-opercular activity when anticipating and receiving task solutions. Unthresholded site-by-site differences in anticipatory neural activity as measured by effect size calculated using average HFB within is of cue onset. Shades of red (the left section of the bar with readings sliding from approximately 0.6 to 1.0) indicate greater response in palatable trials whereas shades of blue (the right section of the bar with readings sliding from approximately −0.6 to −1.0) indicate greater response in taste-neutral trials (FIG. 18A). Same set of plots as in FIG. 18A showing site-by-site differences in receipt neural activity (FIG. 18B). Mean HFB waveforms in exemplar sites that show either discriminative neural activity during anticipation and receipt, or non-discriminatory activity. Time points significantly different between palatable and neutral conditions (p<0.05, cluster-based permutation testing, alpha <0.05) are marked in red along the horizontal axis (such as those lines on top of the traces and indicated by dotted ovals in FIG. 18C), while time points significantly above the pre-cue baseline activity are marked in black (such as those lines on top of the traces along the horizontal axis and indicated by solid ovals in FIG. 18C) (p<0.05, cluster-based permutation testing, alpha <0.05). Transparent lines indicate activity was not different from pre-cue baseline activity. The vertical dashed lines in each column denote the onset of the solution cue (“Cue”, t=0) and solution delivery (“Liquid”, t=3) (FIG. 18C). Error bars show ±SEM.

FIGS. 19A and 19B. Proportion taste-neutral specific channels and effect size in the left anterior and posterior insula. The proportion of cue-specific channels that are favored toward the taste-neutral cue was significantly higher in the left posterior insula (27%) as compared to the left anterior insula (5%) (FIG. 19A). Mean effect size of cue-specific channels in the left anterior insula as compared to the left posterior insula. The left anterior insula had a mean positive effect size, thus favoring the palatable cue. This is significantly different than the mean negative effect size found in the left posterior insula (FIG. 19B).

FIGS. 20A and 20B. Proportion of task-responsive and task-specific electrodes as determined by HFB (70-170 Hz) or gamma (25-50 Hz) activity. Each dot represents a single subject. Proportion of cue-responsive and cue-specific channels out of total channels as indexed by HFB or gamma power. A significantly higher proportion of cue-responsive channels was found using HFB as compared to gamma activity. The proportion of cue-specific channels determined by HFB was also higher than that determined by gamma activity (FIG. 20A). Proportion of receipt-responsive and receipt-specific channels out of total channels as indexed by HFB or gamma power (“Y”). A significantly higher proportion of receipt-responsive channels was found using HFB as compared to gamma activity. The proportion of receipt-specific channels determined by HFB was also higher than that determined by gamma activity. HFB: high-frequency broadband (FIG. 20B).

FIG. 21. Individual subject parcellation of insular and frontal opercular cortices and responses during the milkshake paradigm. Automatic parcellation of the insular cortex was performed using Freesurfer. Electrodes responses were categorized as inactive (grey), cue or receipt-responsive (green, referring to readings sliding from approximately 0.6 to −0.6 in the middle section of the bar at the bottom of each column), or cue or receipt-specific (shades of red or blue respectively, while the red refers to readings sliding from approximately 0.6 to 1.0 in the left section of the bar at the bottom of each column, and the blue refers to readings sliding from approximately −0.6 to −1.0 in the right side of the bar at the bottom of each column).

FIG. 22. Comparison of HFB response onset by anatomical location during task receipt. Histogram showing distribution of ROL of HFB at the visual region, the frontal operculum, the anterior insula, the posterior insula, and visual regions. Each observation represents the ROL of HFB at the single trial level calculated for all trials across all subjects (palatable and neutral conditions). The vertical line in each panel denotes the median ROL time.

FIG. 23. Time-locked HFB activity in the pre-motor region during natural eating stratified by the pre-bite and post-bite (i.e. chewing) periods. Two subjects had superficial electrodes in the pre-motor region (S4, yellow contacts, such as those indicated by dotted ovals, N=4; S7, green contacts, such as those indicated by solid ovals, N=8). For each subject, the mean time-locked waveforms stratified by type of food consumed are shown. Dotted vertical lines in the upper-right and the bottom-right panels denote the immediate time point as food was about to enter the subject's mouth. Time points significantly different between entree versus fruit near the time of food entering the mouth (p<0.05, cluster-based permutation testing, alpha <0.05) are marked in red along the horizontal axis (as lines on top of the traces).

### DETAILED DESCRIPTION

Novel behaviorally relevant neural signatures in both superficial and deep brain regions can serve as distinct neural control signals for the therapeutic modulation of circuitry involved in impaired inhibitory control disorders (ICD). Two target cortical areas include the insula and the hippocampus. Both areas display a similar neurophsyiological signal, delta-theta (˜3-8 Hz) power, which is significantly increased when individuals anticipate feeding of palatable items. Applicants present herein results indicating that the hippocampus is structurally and functionally involved in the feeding circuitry, and that changes in its feeding circuitry connections are observed in obese-binge eaters. Thus, targeting of the hippocampus can be a solution to the feeding circuitry. In addition to the delta-theta waveform, there is a second target signal, gamma, which is recruited in the posterior insula when subjects anticipate palatable items.

Adding to the knowledge of deeper nucleus accumbens (NAc) activity involvement in eating-related disorders, using spectral and new graph-theory network analyses methods, Applicants observed increases in delta local activity and network graph architecture (˜1-4 Hz) to loss of control eating in obese individuals. These methods can be implemented in a closed-loop approach to deliver therapy at the most vulnerable moment in individual suffering from eating-related disorders.

Thus, provided herein, inter alia, are methods for treating impaired inhibitory control disorders. The methods include accessible (both surgically and non-invasively) therapeutic anatomical targets. The methods further include defining the associated electrical activity to detect in these areas, and introduction of novel methods of delivering behavior-specific therapy.

### Definitions

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “disorder” or “disease” as provided herein are used interchangeably and refer to any deviation from the normal health of a mammal and include a state when disease/disorder symptoms are present, as well as conditions in which a deviation (e.g., chemical imbalance, infection, gene mutation, genetic defect, etc.) has occurred, but symptoms are not yet manifested or are not yet fully manifested. According to the present invention, the methods disclosed herein are suitable for use in a patient that is a member of the Vertebrate class, Mammalia, including, without limitation, primates, livestock and domestic pets (e.g., a companion animal). Typically, a patient will be a human patient.

The term “impaired inhibitory control disorder” or “loss of control disorder” as used herein refers to a disordered pattern of behavior characterized by diminished impulse control or compulsions. Impaired inhibitory control disorders (ICD) or loss of control disorders (LOC) include substance abuse, sex addiction or compulsive sexuality, kleptomania, pyromania, trichotillomania, panic disorder, Intermittent Explosive Disorder, compulsive behaviors including gambling, binge eating, night eating, loss of control eating, emotional or stress eating, compulsive eating, purge behaviors, suicidal ideation/attempt and other compulsive behaviors.

Substance abuse refers to compulsive, pathological use of drugs and/or alcohol, including an inability to reduce or prevent consumption. Substance abuse may additionally include impairment in social or occupational functioning as result of substance abuse.

Sex addiction refers to compulsive engagement in sexual activities (e.g., sexual intercourse) despite negative consequences (e.g., negative effects on health, work performance, relationships, or other parts of life).

Compulsive sexuality, also referred to as compulsive sexual behavior, refers to an obsession with sexual thoughts, urges, or behaviors that cause distress and negatively impact or disrupt health, work performance, relationships, or other parts of one's life.

Kleptomania is an impulse control disorder wherein an individual experiences a recurrent urge, and an inability to resist the urge, to steal items which are not needed or have little value. Kleptomania can cause severe emotional pain to the subject and negatively impact relationships.

Pyromania is an impulse control disorder wherein an individual experiences an irresistible impulse to start fires or set fire to objects.

Trichotillomania is an impulse control disorder characterized by a long term urge to pull out one's own hair. Trichotillomania may result in noticeable hair loss. Trichotillomania may also fall within the spectrum of obsessive compulsive disorders.

Panic disorder refers to a type of anxiety disorder wherein an individual experiences recurrent and often unexpected panic attacks. Panic attacks may include heart palpitations or accelerated heart rate, sweating, trembling, sensation of shortness of breath, chest pain or discomfort, nausea or abdominal distress, dizziness, feelings of unreality, fear of losing control, fear of dying, numbness or tingling sensations, and/or chills or hot flushes. An individual suffering from panic disorder may fear the onset of a panic attack, resulting in a change in the person's behavior in an effort to avoid triggering a panic attack.

Intermittent Explosive Disorder (IED) refers to a type of behavioral disorder characterized by explosive outburst of anger and/or violence that are disproportionate to a situation.

Compulsive behaviors contemplated herein include, but are not limited to, gambling characterized by an uncontrollable urge to continue gambling despite negative consequences; eating disorders, such as binge eating which is characterized by recurrent episodes of eating large quantities of food quickly and to the point of discomfort, which may be followed by feelings of depression, disgust, or guilt; night eating which is characterized by a delayed circadian pattern of food intake often accompanied by a sense of shame and/or inability to control one's eating pattern; loss of control eating which is characterized by a sense of loss of control over eating similar to that experienced in binge eating, but not necessarily accompanied by consumption of a large quantity of food; emotional or stress eating which is eating in an effort to alleviate negative emotions; compulsive eating which refers to a compulsion to overeat resulting in consumption of abnormally large quantities of food while simultaneously feeling unable to stop consumption; purge behaviors, for example self-induced vomiting, misuse of laxatives, excessive exercise; suicidal thoughts, also known as suicidal ideation, wherein an individual may consider or formulate plans to kill oneself, and suicidal attempts wherein an individual will engage in a non-fatal, self-directed injurious behavior with the intent of killing oneself.

ICD over eating is common to all binge eaters, and is known to predict poor weight loss following gastric bypass surgery.^{15,31} While this behavior is undoubtedly multifactorial, one of the most obvious environmental factors is the societal overabundance of high-energy, highly refined foods. The reinforcing properties of such food are thought to be mediated by the insular cortex and/or the hippocampus.

Commonly described symptoms of binge eating disorder include frequent dieting and weight loss, hoarding of food, hiding empty food containers, eating late at night, attribution of one's successes and failures to weight, avoiding social situations where food may be present, and feeling depressed or anxious. Binge eating also may cause rapid and unhealthy weight gain (or loss), weight fluctuations, and chronic erratic eating behavior. Binge eating disorder and symptoms associated with binge eating disorder may result in obesity though obesity is not necessarily a result of binge eating disorder. Further, patients with binge eating disorder are often not obese and may even have a below normal weight.

The term “insular cortex”, also referred to as insula or insular lobe, as used herein refers to a portion of the cerebral cortex folded within the lateral sulcus (the fissure separating the temporal lobe from the parietal and frontal lobes) within each hemisphere of the mammalian brain. The insular cortex is believed to be involved in functions linked to emotion or regulation of homeostasis. Functions include compassion, empathy taste, motor control, cognitive function.

The term “hippocampus” as used herein refers to a brain structure located in the temporal lobe. The hippocaampus is associated with consolidation of information from short-term to long-term memory and in spatial memory. The hippocampus is also thought to be involved in approach-avoidance conflict.

The term “brain region(s)” is used according to its plain and ordinary meaning and refers to a brain anatomical region following standard neuroanatomy hierarchies (e.g. a functional, connective or developmental region). Exemplary brain regions include, but are not limited to, brainstem, Medulla oblongata, Medullary pyramids, Olivary body, Inferior olivary nucleus, Rostral ventrolateral medulla, Respiratory center, Dorsal respiratory group, Ventral respiratory group, Pre-Botzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Lateral parabrachial nucleus, Subparabrachial nucleus (Kolliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyrus, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyrus, Middle frontal gyrus, Inferior frontal gyrus, Brodmann areas: 4, 6, 8, 9, 10, 11, 12, 24, 25, 32, 33, 44, 45, 46, 47, Parietal lobe, Parietal cortex, Primary somatosensory cortex (S1), Secondary somatosensory cortex (S2), Posterior parietal cortex, postcentral gyrus, precuneus, Brodmann areas 1, 2, 3 (Primary somesthetic area); 5, 7, 23, 26, 29, 31, 39, 40, Occipital lobe, Primary visual cortex (V1), V2, V3, V4, V5/MT, Lateral occipital gyrus, Cuneus, Brodmann areas 17 (V1, primary visual cortex); 18, 19, temporal lobe, Primary auditory cortex (A1), secondary auditory cortex (A2), Inferior temporal cortex, Posterior inferior temporal cortex, Superior temporal gyrus, Middle temporal gyrus, Inferior temporal gyrus, Entorhinal Cortex, Perirhinal Cortex, Parahippocampal gyrus, Fusiform gyrus, Brodmann areas: 9, 20, 21, 22, 27, 34, 35, 36, 37, 38, 41, 42, Medial superior temporal area (MST), insular cortex, cingulate cortex, Anterior cingulate, Posterior cingulate, dorsal cingulate, Retrosplenial cortex, Indusium griseum, Subgenual area 25, Brodmann areas 23, 24; 26, 29, 30 (retrosplenial areas); 31, 32, cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), and neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract. Brain regions and specific parts of brain regions may be referred to according to their rostral/caudal, dorsal/ventral, medial/lateral, and/or anterior/posterior positions within the brain region with respect to the skull.

The term “brain network”, “brain circuit”, “brain (or neural) connection” or “brain region connectivity” refers to a plurality of brain regions having activity correlated with each other.

The term “brain wave activity” as provided herein refers to a repetitive and/or rhythmic neural activity produced by the central nervous system. Brain wave activity can be detected, for example, through the use of an electrode positioned within brain tissue such that the electrode senses voltage fluctuations driven by neural activity. The structure of voltage fluctuations in brain tissue gives rise to oscillatory activity that can be parsed into different frequencies and/or different frequency bands, wherein each frequency band includes a range of frequencies (e.g., delta band including from about 1 Hz to about 4 Hz). “Low frequency” as provided herein refers to brain wave activity including frequencies within a frequency band spanning between 0 Hz to about 38 Hz.

Non-limiting examples of methods for characterizing brain wave activity include power spectral analyses and cross-frequency coupling measures. Power spectral analysis quantifies the power in each frequency or frequency band per unit time. This analysis allows the power in a particular frequency or frequency band (e.g., low frequency) at a given time (e.g., during or immediately prior to manifestation of a disorder symptom) to be compared against the power in the same frequency or frequency band (e.g., low frequency) at a different period in time (e.g., in the absence of a disorder symptom manifestation), thereby allowing detection of power modulations. Alternatively, changes in power in each frequency band may be visually displayed over time by plotting a spectrogram, thereby allowing detection of changes (e.g., modulations) in power in frequencies or frequency bands of interest (e.g., low frequency) to be analyzed over time (e.g., across time periods including or immediately preceding a symptom manifestation, as well as symptom free time periods).

Cross-frequency coupling measures may be used to describe statistical relationships between frequencies. For example, the phase of low frequency brain wave activity and power of higher frequency (i.e., frequencies faster than those included in low frequency) brain wave activity may have a statistical dependence. Cross-frequency coupling can be assessed at different time points to determine if the statistical dependence of frequencies or frequency bands is modulated by certain conditions (e.g., symptom manifestation).

Brain wave activity may also be related to the activity of individual neurons. A non-limiting example of characterizing the relationship of individual neural activity with brain wave activity is known as spike-field coherence or spike-field coupling. Spike-field coherence quantifies the propensity of action potentials (i.e., spikes) from a given neuron or group of neurons to align with a particular phase of a given frequency of brain wave activity (e.g., low frequency). Spike-field coherence can be assessed at different time points (e.g., periods preceding or concurrent with symptom manifestation and periods temporally distinct from symptom manifestations) such that modulations in spike-field coherence can be determined in response to certain conditions (e.g., symptom manifestation).

As used herein, the term “brain activity level” refers to measurable (e.g., quantifiable) neural activity. Measurable neural activity includes, but is not limited to, a magnitude of activity, a frequency of activity, a delay of activity, or a duration of activity. Brain activity levels may be measured (e.g., quantified) during periods in which no stimulus is presented. In embodiments, the brain activity level measured in the absence of a stimulus is referred to as a baseline brain activity level. Alternatively, brain activity levels may be measured (e.g., quantified) when one or more stimuli are delivered (e.g., an emotional conflict task). In embodiments, the brain activity level measured in the presence of a stimulus is referred to as a brain activity level response. Brain activity levels may be measured simultaneously or sequentially throughout the whole brain, or restricted to specific brain regions (e.g., frontopolar cortex, lateral prefrontal cortex, dorsal anterior cingulate, anterior insula, nucleus accumbens). In embodiments, the brain activity level is determined relative to a baseline brain activity level taken during a baseline period. The baseline period is typically a period during which a stimulus is not presented or has not been presented for a sufficient amount of time (e.g., great than at least 0.05, 0.1, 0.15, 0.25, 0.5, 1, 2, 3, 4, 5, 10, 15, 30, 60 seconds or more). In embodiments, the brain activity level is an oscillatory frequency. In embodiments, the brain activity level is an oscillatory frequency in the ventral striatum. In embodiments, the brain activity level is an oscillatory frequency in the insular cortex and the hippocampus.

A brain activity level may also encompass evaluating functional brain region connectivity. For example, neural activity recorded in a plurality of brain regions may have a specific time course across brain regions that can be correlated to reveal a functional brain connectivity pattern (e.g., at a first time point a first brain regions shows an increase in neural activity and at a second time point a second brain region shows an increase in activity). In embodiments, a brain activity level is a measurement (e.g., quantification) of a time course of neural activity across a plurality of brain regions. In embodiments, a brain activity level is a measurement (e.g., quantification) of a time course of neural activity of one brain region (e.g., insular cortex and/or hippocampus).

It is contemplated that any suitable method of measuring brain activity levels (e.g., neural activity) including, but not limited to, EEG, MEG, fMRI, and fNIRS may be used for practicing the methods described herein, including embodiments thereof.

In embodiments, the frequency of the brain activity level is detected. In embodiments, the brain activity level detected is a frequency of, for example, delta (0.5-4 Hz), theta (5-8 Hz), alpha (8-12 Hz), beta (12-30 Hz), or gamma (30-60 Hz) or a combination thereof. Similarly, the brain activity level can be measured by detecting the amplitude (e.g., power) of oscillations at delta (0.5-4 Hz), theta (5-8 Hz), alpha (8-12 Hz), beta (12-30 Hz), or gamma (30-60 Hz) frequencies or a combination thereof. In embodiments, the brain activity level frequency, amplitude (e.g., power), and phase can be measured. In embodiments, a duration of the brain activity level is measured. In embodiments, a presence or absence of a brain activity level is measured. In embodiments, a brain activity level may be an average brain activity level. In embodiments, a brain activity level may be a median brain activity level.

In embodiments, a brain activity level is an electrical potential or magnetic field recorded from the nervous system, e.g. brain, of a human or other animal, following presentation of a stimulus (e.g., a trial in an emotional conflict task) that is distinct from spontaneous potentials or fields as detected by electroencephalography (EEG), magnetoencephalography (MEG), or other electrophysiological or neurophysiological recording methods. Such potentials and fields are useful for monitoring brain function in health and disease, and, as described herein, may be used for prognostic purposes. The recorded electrical potential or magnetic field is often presented with an amplitude, phase and/or frequency, including the amplitude or power of the response frequency, which generally indicates an intensity and/or pattern of the response.

As used herein, the term “electroencephalography (EEG)” refers to a non-invasive neurophysiological technique that uses an electronic monitoring device to measure and record electrical activity in the brain.

As used herein, the term “magnetoencephalography (MEG)” refers to a non-invasive neurophysiological technique that measures the magnetic fields generated by neuronal activity of the brain. The spatial distributions of the magnetic fields are analyzed to localize the sources of the activity within the brain.

The term “modulate” is used in accordance with its plain ordinary meaning and refers to the act of changing or varying one or more properties. “Modulation” refers to the process of changing or varying one or more properties (e.g., power, cross-frequency coupling, spike-field-coherence). A modulation may be determined by comparing a test sample to a control sample or value.

A “control” sample or value refers to a sample that serves as a reference or baseline, usually a known reference, for comparison to a test sample. For example, a test sample (e.g., low frequency brain wave activity) can be taken from a patient suffering from an ICD during a time period immediately preceding or concurrent with a disorder symptom manifestation (e.g., binge eating) and compared to a sample from the same patient during a period temporally distinct from a symptom manifestation. A control value can be obtained from the same individual, e.g., from an earlier-obtained sample, prior to disease, or prior to treatment. One of skill will recognize that controls can be designed for assessment of any number of parameters.

“Low frequency modulation” as provided herein refers to a change in low frequency brain wave activity (e.g., a change in frequencies between 0 to about 38 Hz) compared to a control. A control may be a baseline low frequency brain wave activity. In embodiments, the baseline low frequency brain wave activity is defined as a time period which is different (longer or shorter (e.g., greater or smaller than 2 seconds)) from the time of manifestation of a disorder symptom. In embodiments, the baseline low frequency brain wave activity is defined as a brain wave frequency different from the frequency characteristic for the manifestation of a disorder symptom. Detection of a low frequency modulation may include methods for characterizing low frequency brain wave activity as described above. Thus, in embodiments, a low frequency modulation is a change in low frequency power relative to a baseline low frequency power. In embodiments, a low frequency modulation is an increase in low frequency power compared to baseline low frequency power. In embodiments, a low frequency modulation is an about 10% to about 45% increase in low frequency power compared to baseline low frequency power. In embodiments, a low frequency modulation is a 10% to 45% increase in low frequency power compared to baseline low frequency power. In embodiments, a low frequency modulation is an about 10% increase in low frequency power compared to baseline low frequency power. In embodiments, a low frequency modulation is a 10% increase in low frequency power compared to baseline low frequency power. In embodiments, a low frequency modulation is an about 15% increase in low frequency power compared to baseline low frequency power. In embodiments, a low frequency modulation is a 15% increase in low frequency power compared to baseline low frequency power. In embodiments, a low frequency modulation is an about 20% increase in low frequency power compared to baseline low frequency power. In embodiments, a low frequency modulation is a 20% increase in low frequency power compared to baseline low frequency power. In embodiments, a low frequency modulation is an about 25% increase in low frequency power compared to baseline low frequency power. In embodiments, a low frequency modulation is a 25% increase in low frequency power compared to baseline low frequency power. In embodiments, a low frequency modulation is an about 30% increase in low frequency power compared to baseline low frequency power. In embodiments, a low frequency modulation is a 30% increase in low frequency power compared to baseline low frequency power. In embodiments, a low frequency modulation is an about 35% increase in low frequency power compared to baseline low frequency power. In embodiments, a low frequency modulation is a 35% increase in low frequency power compared to baseline low frequency power. In embodiments, a low frequency modulation is an about 40% increase in low frequency power compared to baseline low frequency power. In embodiments, a low frequency modulation is a 40% increase in low frequency power compared to baseline low frequency power. In embodiments, a low frequency modulation is an about 45% increase in low frequency power compared to baseline low frequency power. In embodiments, a low frequency modulation is a 45% increase in low frequency power compared to baseline low frequency power.

In embodiments, a low frequency modulation includes a modulation in cross-frequency coupling between low frequency brain wave activity and higher frequency brain wave activity.

In embodiments, a low frequency modulation is a modulation in low frequency spike-field coherence. In embodiments, a low frequency modulation is an increase in low frequency spike-field coherence.

In embodiments, a low frequency modulation precedes the onset of a disorder symptom manifestation. In embodiments, a low frequency modulation precedes the onset of a disorder symptom manifestation by about 2 seconds. In embodiments, a low frequency modulation precedes the onset of a disorder symptom manifestation by 2 seconds. In embodiments, a low frequency modulation precedes the onset of a disorder symptom manifestation by about 1.5 seconds. In embodiments, a low frequency modulation precedes the onset of a disorder symptom manifestation by 1.5 seconds. In embodiments, a low frequency modulation precedes the onset of a disorder symptom manifestation by about 1 second. In embodiments, a low frequency modulation precedes the onset of a disorder symptom manifestation by 1 second. In embodiments, a low frequency modulation precedes the onset of a disorder symptom manifestation by about 0.5 seconds. In embodiments, a low frequency modulation precedes the onset of a disorder symptom manifestation by 0.5 seconds. In embodiments, a low frequency modulation precedes the onset of a disorder symptom manifestation by about 0.1 seconds. In embodiments, a low frequency modulation precedes the onset of a disorder symptom manifestation by 0.1 seconds. Thus, the low frequency modulation is predictive of a disease symptom manifestation (e.g., binge eating). In embodiments, the low frequency modulation is a biomarker.

A “biomarker” as provided herein refers to any assayable characteristics or compositions that are used to identify, predict, or monitor a condition (e.g., symptom of an LOC disorder) or a therapy for said condition in a subject or sample. A biomarker is, for example, a brain wave activity pattern (e.g., low frequency modulation) whose presence is used to identify a condition (e.g. an ICD) or status of a condition (e.g. onset of a disorder symptom manifestation) in a subject or sample. Biomarkers identified herein are measured to determine the onset of disease symptoms and to serve as a trigger for delivering (e.g., administering) a therapeutic stimulation (i.e., electrical stimulation).

The term “electrical stimulation” as used herein refers to an electromagnetic energy administered to the brain in a precise location using an electrode, wherein said electromagnetic energy is capable of modulating an electrical impulse in the brain (e.g., reducing low frequency power in brain region). The electromagnetic energy may be administered at specific parameters which include, for example, frequency, time (burst duration), duty cycle and repetition or any combination thereof. The term “burst duration” as used herein refers to the length of time during which the electrical impulses at a given frequency are administered. Likewise, a “burst” as referred to herein corresponds to the electrical impulse administered at a given frequency. A “duty cycle” as used herein refers to the number and sequence of burst durations (e.g., time-on) followed by the time wherein no burst is administered (e.g., time-off).

As used herein, the term “Transcranial direct current stimulation” refers to a form of neuromodulation that uses constant, low direct current delivered via electrodes on the head. Transcranial direct current stimulation (TDCS) stimulates brain cells by delivering electrical signals and can cause neuron resting membrane potential to depolarize or hyperpolarize.

As used herein, the term “transcranial magnetic stimulation” refers to a noninvasive form of brain stimulation in which a changing magnetic field is used to cause electric current at a specific area of the brain through electromagnetic induction. In transcranial magnetic stimulation (TMS), an electric pulse generator, or stimulator, is connected to a magnetic coil that is connected to the scalp. The stimulator generates a changing electric current within the coil which induces a magnetic field; this field then causes a second inductance of inverted electric charge within the brain.

As used herein, the term “low intensity ultrasound stimulation” refers to low intensity and/or pulsed mechanical waves. Low intensity ultrasound stimulation can be used to non-invasively modulate neuronal activity.

The terms “dose” and “dosage” are used interchangeably herein and are defined by the specific parameters of administering an electrical stimulation. Therefore, a dose as provided herein refers to an electrical stimulus administered at a given frequency, burst duration, duty cycle, repetition or any combination thereof. The dose will vary depending on a number of factors, including the range of normal doses for a given therapy; frequency of administration; size and tolerance of the individual; severity of the condition; and risk of side effects. One of skill will recognize that the dose can be modified depending on the above factors or based on therapeutic progress. In the present invention, the dose may undergo multiple iterations in order to optimize a therapeutic effect.

As used herein, the terms “treat” and “prevent” are not intended to be absolute terms. Treatment can refer to any delay in onset, reduction in the frequency or severity of symptoms, amelioration of symptoms, and/or improvement in patient comfort (e.g., quality of life), etc. The effect of treatment can be compared to the same patient prior to, or after cessation of, treatment.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disorder. Treatment may prevent the disorder from occurring; relieve the disorder's symptoms, fully or partially remove the disorder's underlying cause, shorten a disorder's symptom duration, or do a combination of these things.

“Treating” and “treatment” as used herein include prophylactic treatment. Treatment methods include administering to a subject a therapeutically effective amount of an active agent (i.e., electrical stimulation). The administering step may consist of a single administration or may include a series of administrations. The length of the treatment period depends on a variety of factors, such as the severity of the condition, the age of the patient, the concentration of active agent (e.g., electrical stimulation), the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, electrical stimulations are administered to the subject in an amount and for a duration sufficient to treat the patient.

The term “prevent” refers to a decrease in the occurrence of LOC-associated disorder symptoms in a patient. As indicated above, the prevention may be complete (no detectable symptoms) or partial, such that fewer symptoms are observed than would likely occur absent treatment.

The term “therapeutically effective amount,” as used herein, refers to the amount or dose of a therapeutic agent (i.e., electrical stimulation) sufficient to ameliorate the disorder, as described above. For example, for the given dose, a therapeutically effective amount will show an increase of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

The term “administering” as provided herein, refers to the delivery of an electrical stimulation via one or more electrodes positioned within a specific brain structure (e.g., insular cortex, hippocampus). In the present invention, administration is commenced following detection of a biomarker (e.g., low frequency modulation). In embodiments, administration is accomplished by the apparatus and system provided herein, including embodiments thereof. The same device used to administer electrical stimulation can be used to record brain wave activity to detect a disorder biomarker. In embodiments, administration is triggered automatically by detection of a biomarker (e.g., low frequency modulation). This method of biomarker detection followed by automatic electrical stimulation administration may be referred to herein as “closed-loop” neurostimulation or responsive neurostimulation (RNS). This form of stimulation differs from deep brain stimulation (DBS) in that deep brain stimulation is not a closed-loop system, but rather sends chronic and continuous electrical impulses through the implanted electrodes to specific brain targets. Thus, DBS may be referred to herein as an “open-loop” type of therapeutic treatment, because it involves continuous electrical stimulation that is not preceded by detection of or triggered by specific biomarkers. Where a dose provided herein is compared to a dose administered in DBS, the dose is generally compared to a dose in an open-loop type system.

### Methods

In an aspect is provided a method of detecting a low frequency modulation in the insular cortex and the hippocampus of a subject, wherein the subject is diagnosed with, or suspected of having, an impaired inhibitory control disorder (ICD), the method including: inserting at least one electrode into each the insular cortex and the hippocampus of the subject; and recording brain wave activity in the insular cortex and the hippocampus of the subject.

In embodiments, the low frequency modulation includes a modulation having a frequency between about 0 hertz-8 hertz. In embodiments, the low frequency modulation includes a modulation having a frequency between about 0.25 hertz-8 hertz. In embodiments, the low frequency modulation includes a modulation having a frequency between about 0.5 hertz-8 hertz. In embodiments, the low frequency modulation includes a modulation having a frequency between about 0.75 hertz-8 hertz. In embodiments, the low frequency modulation includes a modulation having a frequency between about 1 hertz-8 hertz. In embodiments, the low frequency modulation includes a modulation having a frequency between about 1.25 hertz-8 hertz. In embodiments, the low frequency modulation includes a modulation having a frequency between about 1.5 hertz-8 hertz. In embodiments, the low frequency modulation includes a modulation having a frequency between about 1.75 hertz-8 hertz. In embodiments, the low frequency modulation includes a modulation having a frequency between about 2 hertz-8 hertz. In embodiments, the low frequency modulation includes a modulation having a frequency between about 2.25 hertz-8 hertz. In embodiments, the low frequency modulation includes a modulation having a frequency between about 2.5 hertz-8 hertz. In embodiments, the low frequency modulation includes a modulation having a frequency between about 2.75 hertz-8 hertz. In embodiments, the low frequency modulation includes a modulation having a frequency between about 3 hertz-8 hertz. In embodiments, the low frequency modulation includes a modulation having a frequency between about 3.25 hertz-8 hertz. In embodiments, the low frequency modulation includes a modulation having a frequency between about 3.5 hertz-8 hertz. In embodiments, the low frequency modulation includes a modulation having a frequency between about 3.75 hertz-8 hertz. In embodiments, the low frequency modulation includes a modulation having a frequency between about 4 hertz-8 hertz. In embodiments, the low frequency modulation includes a modulation having a frequency between about 4.25 hertz-8 hertz. In embodiments, the low frequency modulation includes a modulation having a frequency between about 4.5 hertz-8 hertz. In embodiments, the low frequency modulation includes a modulation having a frequency between about 4.75 hertz-8 hertz. In embodiments, the low frequency modulation includes a modulation having a frequency between about 5 hertz-8 hertz. In embodiments, the low frequency modulation includes a modulation having a frequency between about 5.25 hertz-8 hertz. In embodiments, the low frequency modulation includes a modulation having a frequency between about 5.5 hertz-8 hertz. In embodiments, the low frequency modulation includes a modulation having a frequency between about 5.75 hertz-8 hertz. In embodiments, the low frequency modulation includes a modulation having a frequency between about 6 hertz-8 hertz. In embodiments, the low frequency modulation includes a modulation having a frequency between about 6.25 hertz-8 hertz. In embodiments, the low frequency modulation includes a modulation having a frequency between about 6.5 hertz-8 hertz. In embodiments, the low frequency modulation includes a modulation having a frequency between about 6.75 hertz-8 hertz. In embodiments, the low frequency modulation includes a modulation having a frequency between about 7 hertz-8 hertz. In embodiments, the low frequency modulation includes a modulation having a frequency between about 7.25 hertz-8 hertz. In embodiments, the low frequency modulation includes a modulation having a frequency between about 7.5 hertz-8 hertz. In embodiments, the low frequency modulation includes a modulation having a frequency between about 7.75 hertz-8 hertz.

In embodiments, the low frequency modulation includes a modulation having a frequency between about 0 hertz-7.75 hertz. In embodiments, the low frequency modulation includes a modulation having a frequency between about 0 hertz-7.5 hertz. In embodiments, the low frequency modulation includes a modulation having a frequency between about 0 hertz-7.25 hertz. In embodiments, the low frequency modulation includes a modulation having a frequency between about 0 hertz-7 hertz. In embodiments, the low frequency modulation includes a modulation having a frequency between about 0 hertz-6.75 hertz. In embodiments, the low frequency modulation includes a modulation having a frequency between about 0 hertz-6.5 hertz. In embodiments, the low frequency modulation includes a modulation having a frequency between about 0 hertz-6.25 hertz. In embodiments, the low frequency modulation includes a modulation having a frequency between about 0 hertz-6 hertz. In embodiments, the low frequency modulation includes a modulation having a frequency between about 0 hertz-5.75 hertz. In embodiments, the low frequency modulation includes a modulation having a frequency between about 0 hertz-5.5 hertz. In embodiments, the low frequency modulation includes a modulation having a frequency between about 0 hertz-5.25 hertz. In embodiments, the low frequency modulation includes a modulation having a frequency between about 0 hertz-5 hertz. In embodiments, the low frequency modulation includes a modulation having a frequency between about 0 hertz-4.75 hertz. In embodiments, the low frequency modulation includes a modulation having a frequency between about 0 hertz-4.5 hertz. In embodiments, the low frequency modulation includes a modulation having a frequency between about 0 hertz-4.25 hertz. In embodiments, the low frequency modulation includes a modulation having a frequency between about 0 hertz-4 hertz. In embodiments, the low frequency modulation includes a modulation having a frequency between about 0 hertz-3.75 hertz. In embodiments, the low frequency modulation includes a modulation having a frequency between about 0 hertz-3.5 hertz. In embodiments, the low frequency modulation includes a modulation having a frequency between about 0 hertz-3.25 hertz. In embodiments, the low frequency modulation includes a modulation having a frequency between about 0 hertz-3 hertz. In embodiments, the low frequency modulation includes a modulation having a frequency between about 0 hertz-2.75 hertz. In embodiments, the low frequency modulation includes a modulation having a frequency between about 0 hertz-2.5 hertz. In embodiments, the low frequency modulation includes a modulation having a frequency between about 0 hertz-2.25 hertz. In embodiments, the low frequency modulation includes a modulation having a frequency between about 0 hertz-2 hertz. In embodiments, the low frequency modulation includes a modulation having a frequency between about 0 hertz-1.75 hertz. In embodiments, the low frequency modulation includes a modulation having a frequency between about 0 hertz-1.5 hertz. In embodiments, the low frequency modulation includes a modulation having a frequency between about 0 hertz-1.25 hertz. In embodiments, the low frequency modulation includes a modulation having a frequency between about 0 hertz-1 hertz. In embodiments, the low frequency modulation includes a modulation having a frequency between about 0 hertz-0.75 hertz. In embodiments, the low frequency modulation includes a modulation having a frequency between about 0 hertz-0.5 hertz. In embodiments, the low frequency modulation includes a modulation having a frequency between about 0 hertz-0.25 hertz. In embodiments, the low frequency modulation includes a modulation having a frequency of 0 hertz, 0.25 hertz, 0.5 hertz, 0.75 hertz, 1 hertz, 1.25 hertz, 1.5 hertz, 1.75 hertz, 2 hertz, 2.25 hertz, 2.5 hertz, 2.75 hertz, 3 hertz, 3.25 hertz, 3.5 hertz, 3.75 hertz, 4 hertz, 4.25 hertz, 4.5 hertz, 4.75 hertz, 5 hertz, 5.25 hertz, 5.5 hertz, 5.75 hertz, 6 hertz, 6.25 hertz, 6.5 hertz, 6.75 hertz, 7 hertz, 7.25 hertz, 7.5 hertz, 7.75 hertz, or 8 hertz.

In embodiments, the frequency is a theta brain wave. In embodiments, the low frequency modulation includes a modulation having a frequency between about 3.5 to 7.5 Hz. In embodiments, the low frequency modulation includes a modulation having a frequency between about 3.75 to 7.5 Hz. In embodiments, the low frequency modulation includes a modulation having a frequency between about 4 to 7.5 Hz. In embodiments, the low frequency modulation includes a modulation having a frequency between about 4.25 to 7.5 Hz. In embodiments, the low frequency modulation includes a modulation having a frequency between about 4.5 to 7.5 Hz. In embodiments, the low frequency modulation includes a modulation having a frequency between about 4.75 to 7.5 Hz. In embodiments, the low frequency modulation includes a modulation having a frequency between about 5 to 7.5 Hz. In embodiments, the low frequency modulation includes a modulation having a frequency between about 5.25 to 7.5 Hz. In embodiments, the low frequency modulation includes a modulation having a frequency between about 5.5 to 7.5 Hz. In embodiments, the low frequency modulation includes a modulation having a frequency between about 5.75 to 7.5 Hz. In embodiments, the low frequency modulation includes a modulation having a frequency between about 6 to 7.5 Hz. In embodiments, the low frequency modulation includes a modulation having a frequency between about 6.25 to 7.5 Hz. In embodiments, the low frequency modulation includes a modulation having a frequency between about 6.5 to 7.5 Hz. In embodiments, the low frequency modulation includes a modulation having a frequency between about 6.75 to 7.5 Hz. In embodiments, the low frequency modulation includes a modulation having a frequency between about 7 to 7.5 Hz. In embodiments, the low frequency modulation includes a modulation having a frequency between about 7.25 to 7.5 Hz.

In embodiments, the low frequency modulation includes a modulation having a frequency between about 3.5 to 7.25 Hz. In embodiments, the low frequency modulation includes a modulation having a frequency between about 3.5 to 7 Hz. In embodiments, the low frequency modulation includes a modulation having a frequency between about 3.5 to 6.75 Hz. In embodiments, the low frequency modulation includes a modulation having a frequency between about 3.5 to 6.5 Hz. In embodiments, the low frequency modulation includes a modulation having a frequency between about 3.5 to 6.25 Hz. In embodiments, the low frequency modulation includes a modulation having a frequency between about 3.5 to 6 Hz. In embodiments, the low frequency modulation includes a modulation having a frequency between about 3.5 to 5.75 Hz. In embodiments, the low frequency modulation includes a modulation having a frequency between about 3.5 to 5.5 Hz. In embodiments, the low frequency modulation includes a modulation having a frequency between about 3.5 to 5.25 Hz. In embodiments, the low frequency modulation includes a modulation having a frequency between about 3.5 to 5 Hz. In embodiments, the low frequency modulation includes a modulation having a frequency between about 3.5 to 4.75 Hz. In embodiments, the low frequency modulation includes a modulation having a frequency between about 3.5 to 4.5 Hz. In embodiments, the low frequency modulation includes a modulation having a frequency between about 3.5 to 4.25 Hz. In embodiments, the low frequency modulation includes a modulation having a frequency between about 3.5 to 4 Hz. In embodiments, the low frequency modulation includes a modulation having a frequency between about 3.5 to 3.75 Hz. In embodiments, the low frequency modulation includes a modulation having a frequency of 3.5 Hz, 3.75 Hz, 4 Hz, 4.25 Hz, 4.5 Hz, 4.75 Hz, 5 Hz, 5.25 Hz, 5.5 Hz, 5.75 Hz, 6 Hz, 6.25 Hz, 6.5 Hz, 6.75 Hz, 7 Hz, 7.25 Hz or 7.5 Hz.

In embodiments, the frequency is a delta brain wave. In embodiments, the low frequency modulation includes a modulation having a frequency between about 0.1-4 Hz. In embodiments, the low frequency modulation includes a modulation having a frequency between about 0.4-4 Hz. In embodiments, the low frequency modulation includes a modulation having a frequency between about 0.7-4 Hz. In embodiments, the low frequency modulation includes a modulation having a frequency between about 1-4 Hz. In embodiments, the low frequency modulation includes a modulation having a frequency between about 1.3-4 Hz. In embodiments, the low frequency modulation includes a modulation having a frequency between about 1.6-4 Hz. In embodiments, the low frequency modulation includes a modulation having a frequency between about 1.9-4 Hz. In embodiments, the low frequency modulation includes a modulation having a frequency between about 2.2-4 Hz. In embodiments, the low frequency modulation includes a modulation having a frequency between about 2.5-4 Hz. In embodiments, the low frequency modulation includes a modulation having a frequency between about 2.8-4 Hz. In embodiments, the low frequency modulation includes a modulation having a frequency between about 3.1-4 Hz. In embodiments, the low frequency modulation includes a modulation having a frequency between about 3.4-4 Hz. In embodiments, the low frequency modulation includes a modulation having a frequency between about 3.7-4 Hz.

In embodiments, the low frequency modulation includes a modulation having a frequency between about 0.4-3.7 Hz. In embodiments, the low frequency modulation includes a modulation having a frequency between about 0.4-3.4 Hz. In embodiments, the low frequency modulation includes a modulation having a frequency between about 0.4-3.1 Hz. In embodiments, the low frequency modulation includes a modulation having a frequency between about 0.4-2.8 Hz. In embodiments, the low frequency modulation includes a modulation having a frequency between about 0.4-2.5 Hz. In embodiments, the low frequency modulation includes a modulation having a frequency between about 0.4-2.2 Hz. In embodiments, the low frequency modulation includes a modulation having a frequency between about 0.4-1.9 Hz. In embodiments, the low frequency modulation includes a modulation having a frequency between about 0.4-1.6 Hz. In embodiments, the low frequency modulation includes a modulation having a frequency between about 0.4-1.3 Hz. In embodiments, the low frequency modulation includes a modulation having a frequency between about 0.4-1 Hz. In embodiments, the low frequency modulation includes a modulation having a frequency between about 0.4-0.7 Hz. In embodiments, the low frequency modulation includes a modulation having a frequency of 0.1, 0.4, 0.7, 1, 1.3, 1.6, 1.9, 2.2, 2.5, 2.8, 3.1, 3.4, 3.7 or 4 Hz.

In embodiments, the low frequency modulation is an increase of a frequency between about 0 hertz-8 hertz relative to a standard control. In embodiments, the low frequency modulation is an increase of a frequency between about 0.25 hertz-8 hertz relative to a standard control. In embodiments, the low frequency modulation is an increase of a frequency between about 0.5 hertz-8 hertz relative to a standard control. In embodiments, the low frequency modulation is an increase of a frequency between about 0.75 hertz-8 hertz relative to a standard control. In embodiments, the low frequency modulation is an increase of a frequency between about 1 hertz-8 hertz relative to a standard control. In embodiments, the low frequency modulation is an increase of a frequency between about 1.25 hertz-8 hertz relative to a standard control. In embodiments, the low frequency modulation is an increase of a frequency between about 1.5 hertz-8 hertz relative to a standard control. In embodiments, the low frequency modulation is an increase of a frequency between about 1.75 hertz-8 hertz relative to a standard control. In embodiments, the low frequency modulation is an increase of a frequency between about 2 hertz-8 hertz relative to a standard control. In embodiments, the low frequency modulation is an increase of a frequency between about 2.25 hertz-8 hertz relative to a standard control. In embodiments, the low frequency modulation is an increase of a frequency between about 2.5 hertz-8 hertz relative to a standard control. In embodiments, the low frequency modulation is an increase of a frequency between about 2.75 hertz-8 hertz relative to a standard control. In embodiments, the low frequency modulation is an increase of a frequency between about 3 hertz-8 hertz relative to a standard control. In embodiments, the low frequency modulation is an increase of a frequency between about 3.25 hertz-8 hertz relative to a standard control. In embodiments, the low frequency modulation is an increase of a frequency between about 3.5 hertz-8 hertz relative to a standard control. In embodiments, the low frequency modulation is an increase of a frequency between about 3.75 hertz-8 hertz relative to a standard control. In embodiments, the low frequency modulation is an increase of a frequency between about 4 hertz-8 hertz relative to a standard control. In embodiments, the low frequency modulation is an increase of a frequency between about 4.25 hertz-8 hertz relative to a standard control. In embodiments, the low frequency modulation is an increase of a frequency between about 4.5 hertz-8 hertz relative to a standard control. In embodiments, the low frequency modulation is an increase of a frequency between about 4.75 hertz-8 hertz. In embodiments, the low frequency modulation is an increase of a frequency between about 5 hertz-8 hertz. In embodiments, the low frequency modulation is an increase of a frequency between about 5.25 hertz-8 hertz relative to a standard control. In embodiments, the low frequency modulation is an increase of a frequency between about 5.5 hertz-8 hertz relative to a standard control. In embodiments, the low frequency modulation is an increase of a frequency between about 5.75 hertz-8 hertz relative to a standard control. In embodiments, the low frequency modulation is an increase of a frequency between about 6 hertz-8 hertz relative to a standard control. In embodiments, the low frequency modulation is an increase of a frequency between about 6.25 hertz-8 hertz relative to a standard control. In embodiments, the low frequency modulation is an increase of a frequency between about 6.5 hertz-8 hertz relative to a standard control. In embodiments, the low frequency modulation is an increase of a frequency between about 6.75 hertz-8 hertz relative to a standard control. In embodiments, the low frequency modulation is an increase of a frequency between about 7 hertz-8 hertz relative to a standard control. In embodiments, the low frequency modulation is an increase of a frequency between about 7.25 hertz-8 hertz relative to a standard control. In embodiments, the low frequency modulation is an increase of a frequency between about 7.5 hertz-8 hertz relative to a standard control. In embodiments, the low frequency modulation is an increase of a frequency between about 7.75 hertz-8 hertz relative to a standard control.

In embodiments, the low frequency modulation is an increase of a frequency between about 0 hertz-7.75 hertz relative to a standard control. In embodiments, the low frequency modulation is an increase of a frequency between about 0 hertz-7.5 hertz relative to a standard control. In embodiments, the low frequency modulation is an increase of a frequency between about 0 hertz-7.25 hertz relative to a standard control. In embodiments, the low frequency modulation is an increase of a frequency between about 0 hertz-7 hertz relative to a standard control. In embodiments, the low frequency modulation is an increase of a frequency between about 0 hertz-6.75 hertz relative to a standard control. In embodiments, the low frequency modulation is an increase of a frequency between about 0 hertz-6.5 hertz relative to a standard control. In embodiments, the low frequency modulation is an increase of a frequency between about 0 hertz-6.25 hertz relative to a standard control. In embodiments, the low frequency modulation is an increase of a frequency between about 0 hertz-6 hertz relative to a standard control. In embodiments, the low frequency modulation is an increase of a frequency between about 0 hertz-5.75 hertz relative to a standard control. In embodiments, the low frequency modulation is an increase of a frequency between about 0 hertz-5.5 hertz relative to a standard control. In embodiments, the low frequency modulation is an increase of a frequency between about 0 hertz-5.25 hertz relative to a standard control. In embodiments, the low frequency modulation is an increase of a frequency between about 0 hertz-5 hertz relative to a standard control. In embodiments, the low frequency modulation is an increase of a frequency between about 0 hertz-4.75 hertz relative to a standard control. In embodiments, the low frequency modulation is an increase of a frequency between about 0 hertz-4.5 hertz relative to a standard control. In embodiments, the low frequency modulation is an increase of a frequency between about 0 hertz-4.25 hertz relative to a standard control. In embodiments, the low frequency modulation is an increase of a frequency between about 0 hertz-4 hertz relative to a standard control. In embodiments, the low frequency modulation is an increase of a frequency between about 0 hertz-3.75 hertz relative to a standard control. In embodiments, the low frequency modulation is an increase of a frequency between about 0 hertz-3.5 hertz relative to a standard control. In embodiments, the low frequency modulation is an increase of a frequency between about 0 hertz-3.25 hertz relative to a standard control. In embodiments, the low frequency modulation is an increase of a frequency between about 0 hertz-3 hertz relative to a standard control. In embodiments, the low frequency modulation is an increase of a frequency between about 0 hertz-2.75 hertz relative to a standard control. In embodiments, the low frequency modulation is an increase of a frequency between about 0 hertz-2.5 hertz relative to a standard control. In embodiments, the low frequency modulation is an increase of a frequency between about 0 hertz-2.25 hertz relative to a standard control. In embodiments, the low frequency modulation is an increase of a frequency between about 0 hertz-2 hertz relative to a standard control. In embodiments, the low frequency modulation is an increase of a frequency between about 0 hertz-1.75 hertz relative to a standard control. In embodiments, the low frequency modulation is an increase of a frequency between about 0 hertz-1.5 hertz relative to a standard control. In embodiments, the low frequency modulation is an increase of a frequency between about 0 hertz-1.25 hertz relative to a standard control. In embodiments, the low frequency modulation is an increase of a frequency between about 0 hertz-1 hertz relative to a standard control. In embodiments, the low frequency modulation is an increase of a frequency between about 0 hertz-0.75 hertz relative to a standard control. In embodiments, the low frequency modulation is an increase of a frequency between about 0 hertz-0.5 hertz relative to a standard control. In embodiments, the low frequency modulation is an increase of a frequency between about 0 hertz-0.25 hertz relative to a standard control. In embodiments, the low frequency modulation is an increase of a frequency of 0 hertz, 0.25 hertz, 0.5 hertz, 0.75 hertz, 1 hertz, 1.25 hertz, 1.5 hertz, 1.75 hertz, 2 hertz, 2.25 hertz, 2.5 hertz, 2.75 hertz, 3 hertz, 3.25 hertz, 3.5 hertz, 3.75 hertz, 4 hertz, 4.25 hertz, 4.5 hertz, 4.75 hertz, 5 hertz, 5.25 hertz, 5.5 hertz, 5.75 hertz, 6 hertz, 6.25 hertz, 6.5 hertz, 6.75 hertz, 7 hertz, 7.25 hertz, 7.5 hertz, 7.75 hertz, or 8 hertz relative to a standard control.

In embodiments, the low frequency modulation is an increase of a frequency between about 3.5 to 7.5 Hz relative to a standard control. In embodiments, the low frequency modulation is an increase of a frequency between about 3.75 to 7.5 Hz relative to a standard control. In embodiments, the low frequency modulation is an increase of a frequency between about 4 to 7.5 Hz relative to a standard control. In embodiments, the low frequency modulation is an increase of a frequency between about 4.25 to 7.5 Hz relative to a standard control. In embodiments, the low frequency modulation is an increase of a frequency between about 4.5 to 7.5 Hz relative to a standard control. In embodiments, the low frequency modulation is an increase of a frequency between about 4.75 to 7.5 Hz relative to a standard control. In embodiments, the low frequency modulation is an increase of a frequency between about 5 to 7.5 Hz relative to a standard control. In embodiments, the low frequency modulation is an increase of a frequency between about 5.25 to 7.5 Hz relative to a standard control. In embodiments, the low frequency modulation is an increase of a frequency between about 5.5 to 7.5 Hz relative to a standard control. In embodiments, the low frequency modulation is an increase of a frequency between about 5.75 to 7.5 Hz relative to a standard control. In embodiments, the low frequency modulation is an increase of a frequency between about 6 to 7.5 Hz relative to a standard control. In embodiments, the low frequency modulation is an increase of a frequency between about 6.25 to 7.5 Hz relative to a standard control. In embodiments, the low frequency modulation is an increase of a frequency between about 6.5 to 7.5 Hz relative to a standard control. In embodiments, the low frequency modulation is an increase of a frequency between about 6.75 to 7.5 Hz relative to a standard control. In embodiments, the low frequency modulation is an increase of a frequency between about 7 to 7.5 Hz relative to a standard control. In embodiments, the low frequency modulation is an increase of a frequency between about 7.25 to 7.5 Hz relative to a standard control.

In embodiments, the low frequency modulation is an increase of a frequency between about 3.5 to 7.5 Hz relative to a standard control. In embodiments, the low frequency modulation is an increase of a frequency between about 3.5 to 7.25 Hz relative to a standard control. In embodiments, the low frequency modulation is an increase of a frequency between about 3.5 to 7 Hz relative to a standard control. In embodiments, the low frequency modulation is an increase of a frequency between about 3.5 to 6.75 Hz relative to a standard control. In embodiments, the low frequency modulation is an increase of a frequency between about 3.5 to 6.5 Hz relative to a standard control. In embodiments, the low frequency modulation is an increase of a frequency between about 3.5 to 6.25 Hz relative to a standard control. In embodiments, the low frequency modulation is an increase of a frequency between about 3.5 to 6 Hz relative to a standard control. In embodiments, the low frequency modulation is an increase of a frequency between about 3.5 to 5.75 Hz relative to a standard control. In embodiments, the low frequency modulation is an increase of a frequency between about 3.5 to 5.5 Hz relative to a standard control. In embodiments, the low frequency modulation is an increase of a frequency between about 3.5 to 5.25 Hz relative to a standard control. In embodiments, the low frequency modulation is an increase of a frequency between about 3.5 to 5 Hz relative to a standard control. In embodiments, the low frequency modulation is an increase of a frequency between about 3.5 to 4.75 Hz relative to a standard control. In embodiments, the low frequency modulation is an increase of a frequency between about 3.5 to 4.5 Hz relative to a standard control. In embodiments, the low frequency modulation is an increase of a frequency between about 3.5 to 4.25 Hz relative to a standard control. In embodiments, the low frequency modulation is an increase of a frequency between about 3.5 to 4 Hz relative to a standard control. In embodiments, the low frequency modulation is an increase of a frequency between about 3.5 to 3.75 Hz relative to a standard control. In embodiments, the low frequency modulation is an increase of a frequency of 3.5 Hz, 3.75 Hz, 4 Hz, 4.25 Hz, 4.5 Hz, 4.75 Hz, 5 Hz, 5.25 Hz, 5.5 Hz, 5.75 Hz, 6 Hz, 6.25 Hz, 6.5 Hz, 6.75 Hz, 7 Hz, 7.25 Hz, or 7.5 Hz relative to a standard control.

In embodiments, the low frequency modulation is an increase of a frequency between about 0.1-4 Hz relative to a standard control. In embodiments, the low frequency modulation is an increase of a frequency between about 0.4-4 Hz relative to a standard control. In embodiments, the low frequency modulation is an increase of a frequency between about 0.7-4 Hz relative to a standard control. In embodiments, the low frequency modulation is an increase of a frequency between about 1-4 Hz relative to a standard control. In embodiments, the low frequency modulation is an increase of a frequency between about 1.3-4 Hz relative to a standard control. In embodiments, the low frequency modulation is an increase of a frequency between about 1.6-4 Hz relative to a standard control. In embodiments, the low frequency modulation is an increase of a frequency between about 1.9-4 Hz relative to a standard control. In embodiments, the low frequency modulation is an increase of a frequency between about 2.2-4 Hz relative to a standard control. In embodiments, the low frequency modulation is an increase of a frequency between about 2.5-4 Hz relative to a standard control. In embodiments, the low frequency modulation is an increase of a frequency between about 2.8-4 Hz relative to a standard control. In embodiments, the low frequency modulation is an increase of a frequency between about 3.1-4 Hz relative to a standard control. In embodiments, the low frequency modulation is an increase of a frequency between about 3.4-4 Hz relative to a standard control. In embodiments, the low frequency modulation is an increase of a frequency between about 3.7-4 Hz relative to a standard control.

In embodiments, the low frequency modulation is an increase of a frequency between about 0.4-3.7 Hz relative to a standard control. In embodiments, the low frequency modulation is an increase of a frequency between about 0.4-3.4 Hz relative to a standard control. In embodiments, the low frequency modulation is an increase of a frequency between about 0.4-3.1 Hz relative to a standard control. In embodiments, the low frequency modulation is an increase of a frequency between about 0.4-2.8 Hz relative to a standard control. In embodiments, the low frequency modulation is an increase of a frequency between about 0.4-2.5 Hz relative to a standard control. In embodiments, the low frequency modulation is an increase of a frequency between about 0.4-2.2 Hz relative to a standard control. In embodiments, the low frequency modulation is an increase of a frequency between about 0.4-1.9 Hz relative to a standard control. In embodiments, the low frequency modulation is an increase of a frequency between about 0.4-1.6 Hz relative to a standard control. In embodiments, the low frequency modulation is an increase of a frequency between about 0.4-1.3 Hz relative to a standard control. In embodiments, the low frequency modulation is an increase of a frequency between about 0.4-1 Hz relative to a standard control. In embodiments, the low frequency modulation is an increase of a frequency between about 0.4-0.7 Hz relative to a standard control. In embodiments, the low frequency modulation is an increase of a frequency of 0.1, 0.4, 0.7, 1, 1.3, 1.6, 1.9, 2.2, 2.5, 2.8, 3.1, 3.4, 3.7, or 4 Hz relative to a standard control.

For the methods provided herein, in embodiments, the subject suffers from refractory eating-related disorder. In embodiments, the refractory eating-related disorder is binge eating.

In embodiments, the frequency is increased when the subject is contacted with a visceral or a gustatory input. In embodiments, the frequency is increased when the subject is contacted with a visceral input. In embodiments, the frequency is increased when the subject is contacted with a gustatory input. In embodiments, the frequency is increased when the subject anticipates feeding of a palatable item.

In embodiments, the standard control is frequency in the absence of a visceral or a gustatory input. In embodiments, the standard control is frequency in the absence of a visceral input. In embodiments, the standard control is frequency in the absence of a gustatory input.

For the methods provided herein, in embodiments, the ICD includes a disorder that is associated with a lack of impulse control, and wherein the ICD includes one or more of substance abuse, sex addiction or compulsive sexuality, kleptomania, pyromania, trichotillomania, panic disorder, Intermittent Explosive Disorder, compulsive behaviors including gambling, binge eating, night eating, loss of control eating, emotional or stress eating, compulsive eating, purge behaviors, or suicidal ideation/attempt. In embodiments, the ICD is substance abuse. In embodiments, the ICD is sex addiction. In embodiments, the ICD is compulsive sexuality. In embodiments, the ICD is kleptomania. In embodiments, the ICD is pyromania. In embodiments, the ICD is trichotillomania. In embodiments, the ICD is panic disorder. In embodiments, the ICD is Intermittent Explosive Disorder. In embodiments, the ICD is a compulsive behavior. In embodiments, the compulsive behaviors include gambling, binge eating, night eating, loss of control eating, emotional or stress eating, compulsive eating, purge behaviors, or suicidal ideation/attempt. In embodiments, the compulsive behavior is gambling. In embodiments, the compulsive behavior is binge eating. In embodiments, the compulsive behavior is night eating. In embodiments, the compulsive behavior is loss of control eating. In embodiments, the compulsive behavior is emotional eating. In embodiments, the compulsive behavior is stress eating. In embodiments, the compulsive behavior is compulsive eating. In embodiments, the compulsive behavior is purge behaviors. In embodiments, the compulsive behavior is suicidal ideation/attempt.

In embodiments, the method further includes administering, in response to the low frequency modulation, a stimulation to the insular cortex and the hippocampus of the subject.

In embodiments, the stimulation is transcranial direct current stimulation (TDCS), transcranial magnetic stimulation (TMS), or low intensity ultrasound stimulation. In embodiments, the stimulation is transcranial direct current stimulation (TDCS). In embodiments, the stimulation is transcranial magnetic stimulation (TMS). In embodiments, the stimulation is low intensity ultrasound stimulation.

In embodiments, the stimulation is an electrical stimulation. In embodiments, a dose of the electrical stimulation is less than a dose corresponding to deep brain stimulation. In embodiments, the frequency of the electrical stimulation is 5 hertz, 10 hertz, 12 hertz, 160 hertz, 212 hertz, or 333 hertz. In embodiments, the frequency of the electrical stimulation is 5 hertz. In embodiments, the frequency of the electrical stimulation is 10 hertz. In embodiments, the frequency of the electrical stimulation is 12 hertz. In embodiments, the frequency of the electrical stimulation is 160 hertz. In embodiments, the frequency of the electrical stimulation is 212 hertz. In embodiments, the frequency of the electrical stimulation is 333 hertz.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Evaluation of Responsive Neurostimulation of Nucleus Accumbens (NAc) in Patients with Severe Loss of Control (LOC) Eating

Introduction

Loss of control (LOC) eating is common in obesity and complicates treatments in all binge eaters. Animal studies, as well as neuroimaging studies in humans, have provided insight into the neuroanatomy of LOC eating. That LOC eating is a neuropsychiatric disorder is demonstrated by the modest success of cognitive-behavioral therapy (CBT) and weight-control medications that target neurotransmitters in brain regions that regulate food intake. Bariatric surgery is the most effective treatment for refractory morbid obesity, but does not work for all patients. Applicants previously examined patient characteristics that predict unsuccessful postsurgical outcomes. Only preoperative variables were found to predict suboptimal surgical outcomes and one was poor dietary adherence, including LOC eating.

Methods provided herein address the clinical need for a new treatment for obese subjects with LOC eating. The propensity for uncontrolled eating is multifactorial, with risk factors including adverse mood states such as anxiety and even hedonic properties of highly palatable, calorically dense food, mediated by the dopamine system's projections from the ventral tegmental area to the nucleus accumbens (NAc). Moreover, obesity has been associated with decreased dopamine type 2 receptor availability in portions of the striatum, including the NAc, which may predispose to LOC eating to compensate for attenuated dopamine signaling.

Electrical stimulation targeting small regions in the brain such as the NAc is reported to be safe and efficacious for other compulsive-like disorders. In fact, one obese patient with comorbid obsessive compulsive disorder successfully stopped smoking and lost weight after NAc stimulation, supporting common circuitry underlying the processing of diverse rewards. Before this can be applied to LOC eating in obese humans, however, nonclinical studies are important to assess potential efficacy and troubleshoot different stimulation approaches.

High frequency electrical stimulation of the NAc in mice blocks binge-like eating behaviors and induces weight loss in obese mice was found. In studies proposed here, Detection algorithm needs were tested of an available neurostimulation system in a limited cohort of human subjects to ensure software capabilities of the implanted system are optimized. In view of prior preclinical studies, treatment strategy was designed for LOC overeating in obese humans who have not responded to other treatments. These individuals will be treated in the NAc with responsive neurostimulation, which could have greater efficacy, safety and tolerability than continuous stimulation.

A human condition was identified that is extremely medically underserved. A novel therapeutic approach is proposed with an off-the-shelf, currently available system that is supported by both basic and translational science investigations. It is proposed to only modify the software of this existing sensing technology should the current device not be capable of detecting an LFP-biomarker of LOC eating. The device to be developed would be the “Research RNS” (rRNS) neurostimulator. This device will build upon the experience gained with NeuroPace's RNS System that is FDA approved for treatment of medically intractable partial onset seizures. The rRNS System will use NeuroPace's current hardware platform for which new indication-specific software features may be developed. A major benefit of adapting the RNS System to provide responsive stimulation for this novel indication is that there is already extensive human safety experience with the existing device. The RNS neurostimulator and NeuroPace leads have been implanted in close to 700 epilepsy patients (clinical trial+post-approval). Long-term results for the 256 patients who participated in the epilepsy clinical trials demonstrate durable seizure reductions and long-term safety with an average follow-up of 7 years as of February 2016. Like the RNS System, the rRNS System will provide a user-friendly opportunity to obtain chronic recordings of the brain while providing a therapeutic intervention for a pathologic eating behavior in obesity.

Closed-loop, or responsive, stimulation may provide clinical advantages over open-loop stimulation, especially for episodic disorders such as disordered eating. Open-loop stimulation typically delivers continuous or scheduled stimulation, which results in high duty cycles and significant energy requirements that use large batteries and therefore larger devices. Closed-loop (responsive) systems such as the RNS System are power efficient because stimulation is provided only when needed. For example, for the treatment of epilepsy, the RNS neurostimulator typically delivers less than 5 mins of stimulation in total per day, or 0.3% of the 1,440 mins of stimulation per day delivered by open-loop DBS. Responsive stimulation as provided by the RNS System incorporates signal processing, pattern detection, and decision-making capabilities. The data provided by the neurostimulator can be used by the clinician to monitor the electrophysiological state and electrophysiological response to stimulation. Responsive stimulation is therefore likely to better meet the needs of patients and open the possibility of using electrophysiological markers to direct treatment in real time, rather than waiting for the patient to display and report symptoms.

A major goal of this investigation is to acquire chronic ambulatory neural data from the brain's salience system. The sensing and signal processing capabilities of the RNS neurostimulator will be used to acquire and store neural data from patients in real life settings. This chronic data is likely to be more representative of the physiological state than acute recordings, which are impacted by the implant procedure for as long as several months after implantation. Long-term longitudinal neural data collected from individual patients will contain a wealth of information about neurophysiological biomarkers, dynamic changes in disease state, and electrophysiological responses to treatment. These data will be analyzed to gain a better understanding of the brain, define the requirements and guide the evolution of neurotechnologies, and to develop personalized treatment strategies for individual patients. Eventually, the general public will benefit from a better understanding of the brain and the availability of improved medical care.

The Brain Research through Advancing Innovative Neurotechnologies (B.R.A.I.N.) Initiative highlights the need for new innovative neurotechnologies that will revolutionize the understanding of the human brain and accelerate the development of new medical treatments for neurological disorders. This project meets that intent and is comprised of a team that can successfully accomplish its goals. The investigators have demonstrated expertise in a nonclinical model of obesity, in development of a responsive neurostimulator, in successful conduct of feasibility and pivotal trials of a Class III medical device in a large cohort of patients with epilepsy, in successful submission and FDA approval of a PMA, and in clinical treatment of patients with severe obesity. Close work with the NIH and FDA provides confidence to successfully complete the technology development early clinical work to establish whether responsive stimulation should be taken into further investigational trials as a therapy for humans suffering from LOC overeating and refractory obesity.

There are compelling reasons to evaluate new approaches to optimizing long-term weight control and quality of life in the obese population where eating disorders are prevalent and destructive. The preliminary non-clinical data support the use of brain stimulation that targets reward circuitry to ameliorate LOC eating behaviors. Initial human testing will be performed with a chronically implanted device to best examine potential feasibility and safety of this novel therapeutic strategy for a major unmet medical need.

The beneficiaries of this work include patients with treatment refractory obesity and LOC eating that lead to noncompliance with dietary and behavioral regimens. The majority of obese patients will report histories of LOC eating, ranging from overeating and compulsive binge eating to emotional eating and night eating, all of which are strongly related to cravings for calorically dense foods. Forty percent of these individuals will meet formal criteria for binge eating disorder or food addiction. Although binge eating may be physically impossible following bariatric surgery due to the physical constraints of a gastric sleeve or pouch, a different facet of binge-eating pathology with persistent sensations of LOC may remain or even emerge postoperatively. Indeed, consumption of large portions of high-fat foods after bariatric surgery typically results in vomiting and/or dumping syndrome. Recent research suggests that LOC eating is clinically meaningful due to the caloric density of the chosen foods, regardless of the amount of food consumed. For these reasons, LOC eating is the phenotype in obesity which is targeted by this therapy. It has tremendous relevance to obese patients both before and after bariatric surgery and it is a well-established construct with a validated scale and a direct causal relationship with eating disorders common to obesity.

Multiple studies have examined the impact of pathologic eating habits on patients' ability to comply with weight loss measures and outcomes of bariatric surgery. Specifically, the subjective sense of LOC is what obese subjects experience prior to these pathological eating behaviors. LOC eating is a negative prognostic indicator for weight loss, both at short and long-term follow-up. In fact, 50% of patients with LOC eating before bariatric surgery exhibited the same disorder postoperatively. LOC also significantly impairs quality of life due to patients' inability to manage their eating patterns. Associations with other psychiatric comorbidities, including depression, are reported, as well as with metabolic syndrome and obesity.

A treatment that intervenes before a pathological behavior occurs offers tremendous advantages to patients. The guilt and shame after a LOC meal could be avoided if an intervention prevented that behavior from occurring. There is an opportunity for such an intervention for pathological episodic eating. Nonclinical work provides strong evidence that these behaviors arise from the brain reward circuitry and discrete anatomic targets are identified that can be targeted for episodic stimulation. Because patients experience an urge before the LOC, stimulation can be triggered by the patient. Nonclinical data suggest that an electrophysiological biomarker to trigger responsive stimulation can be identified as well.

Beneficiaries of this technology also include the clinical and scientific communities as well as the general public due to new knowledge and insight into the neuroanatomy and neurophysiology of LOC eating in obesity, new neurotechnology, and the availability of new medical treatments. Users of the proposed medical device technology will include patients, scientists, clinicians, and caregivers who interact directly with the RNS System. Design features for the RNS neurostimulator are driven by the needs of the users and beneficiaries. In general, these users require a device that is safe, robust, easy to use, and affordable. The clinical and scientific communities require useful and comprehensive knowledge, while the general public desires effective and intuitive medical treatments. In past decades, brain implants were viewed suspiciously in the media and popular literature and some of these misgivings still remain. With the pervasiveness of handheld computing devices, the growing interest in wearable personal monitors and the positive response earned by the approved RNS System, closed-loop neurostimulation therapy is likely to generate wide interest. Last, the medical device industry and U.S. economy will benefit from new scientific knowledge and from the development of a new therapeutic technology that is safe, effective, and readily acceptable.

Many patients with obesity and co-morbid pathologic eating disorders are suboptimally treated. At present, CBT and gastric bypass surgery are 2 therapeutic options that can provide significant benefit to some but not all of this patient population. CBT does not have long-term effects, and gastric bypass surgery is highly morbid and may be limited in its long-term durability for eating disorders in obesity. The subset of patients who are nonresponders or have recurrent or persistent disease or complications of therapy may require escalation of therapy, correction of surgical complications, or a new treatment modality. Thus, what is needed for patients with obesity and pathological eating who do not respond to traditional therapies is a safe, alternative treatment option such as a direct brain-targeted, non-lesional, non-respective therapy, as is the case with the RNS System. The RNS neurostimulator will also provide the unique opportunity to record from the human brain with a chronically implanted and internalized system found to be safe over years of follow-up as well as effective at long-term storage of electrographic data. The RNS System is expected to provide patients with real-time control over their eating disorder by empowering them or their caregivers to trigger stimulation to the NAc with a magnet swipe, and to allow clinicians to monitor brain recordings remotely to examine how the brain responds or changes to exposures to calorically dense food. This quantitative data could be more sensitive and informative than the EMA that is the current standard of care.

The project described herein is designed to: 1) explore if responsive stimulation of the NAc can serve as a treatment for LOC eating associated with obesity; 2) identify electrophysiological signals associated with the pathologic episodic behavior that could serve as a biomarker that triggers stimulation delivery; 3) optimize the technology's software needed to achieve the therapeutic goal; and 4) determine whether this responsive stimulation is sufficiently safe and tolerated; and 5) show enough evidence for potential efficacy to warrant industry investment in a full development and clinical trial program leading to regulatory approval for this indication for clinical use.

The RNS® System is available for treating epilepsy with closed-loop responsive neurostimulation, and based on animal and human studies that predated this application, it appears to have a wide range of capabilities that may be required in this clinical application. Should no LFP-biomarker be detected in the initial study, NeuroPace will develop the “Research RNS” (rRNS) neurostimulator. The rRNS neurostimulator will utilize the same mechanical design and hardware as the currently approved RNS.

The RNS System has been designed to be easily used by the physician and patient. The physician uses a wand held over the implanted neurostimulator to transfer data to a physician used programmer. Data includes information regarding electrode impedance and battery voltage, time and date stamped records of all detections and stimulations, and the history of all detection and stimulation programmings. The physician reviews electrophysiological data stored by the neurostimulator. Using the NeuroPace detection algorithms, doctors program the device to detect the electrical patterns that appear to predict LOC related eating behaviors, and then program the stimulation settings. In the early feasibility study, patients will have a magnet to swipe over the neurostimulator in order to store an LFP record and to trigger stimulation to the NAc. This will be used by each subject when a craving occurs or LOC is anticipated.

The RNS System has been designed to be easily used by the physician and patient. The physician uses a wand held over the implanted neurostimulator to transfer data to a physician used programmer. Data includes information regarding electrode impedance and battery voltage, time and date stamped records of all detections and stimulations, and the history of all detection and stimulation programmings. The physician reviews electrophysiological data stored by the neurostimulator. Using the NeuroPace detection algorithms, doctors program the device to detect the electrical patterns that appear to predict LOC related eating behaviors, and then program the stimulation settings. In the early feasibility study, patients will have a magnet to swipe over the neurostimulator in order to store an LFP record and to trigger stimulation to the NAc. This will be used by each subject when a craving occurs or LOC is anticipated.

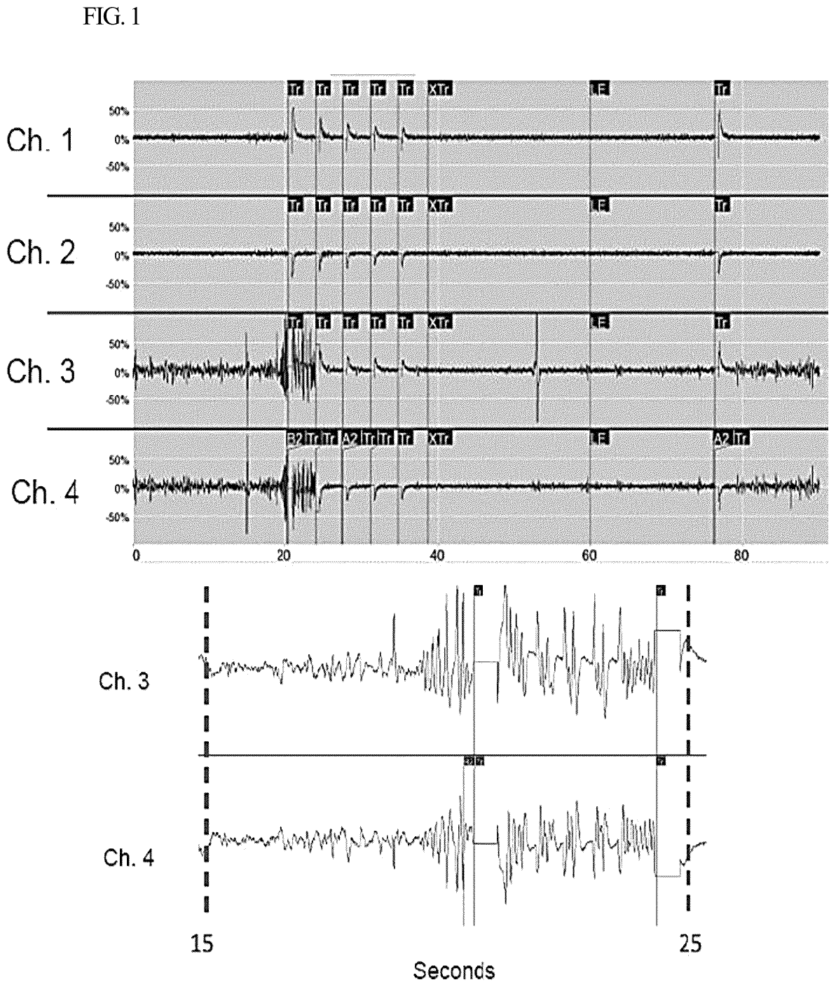

A feature of the RNS System is the internet accessible patient data repository, or PDMS. Data from every patient implanted with the neurostimulator are transferred from the programmer to the secure PDMS database for storage. In addition, all patients have a remote monitor, which allows them to obtain data from their neurostimulator in between clinic visits to transfer this data to the PDMS over the internet or a telephone line (the remote monitor cannot be used to program the neurostimulator). Clinicians with the appropriate credentials access the PDMS to review the neurostimulator data for their patients. New detection settings are simulated on stored electrophysiological data and can be transmitted directly to the programmer to use at the patient's next office visit. This facilitates data acquisition, data storage and data review. The programmer, remote monitor and the PDMS have been successfully utilized throughout the clinical development of the RNS System as well as in commercial use. An example of an LFP display on the PDMS from an epilepsy patient is shown in FIG. 1. The overarching goal of this closed-loop system is to let doctors take their expert knowledge—their ability to evaluate a patient's condition and adjust therapy accordingly—and embed it in an implanted device. The rRNS neurostimulator system if needed in this study will react faster than current devices, provide more tailored therapy to individuals, and provide more information to clinicians, including how physiological states relate to clinical outcomes. From this information, scientists and engineers hope to learn more about how the reward circuitry works, how it is affected by disease, and how to design better treatments. In addition, studies will examine whether patient controlled magnet triggered stimulation can block LOC overeating. Enhancing patient empowerment improves outcomes in nearly all facets of medicine.

Methods and Results

LOC eating is common to all binge eaters, and has been reported to predict 50% of failed bariatric surgery cases. While the cause of aberrant behavior is multifactorial, one of the most obvious factors is societal overabundance of high-energy, highly palatable foods. The reinforcing properties of such food are thought to be mediated by the NAc, a striatal brain region central to regulating goal-directed actions toward salient stimuli. Delivering electrical stimulation to the NAc immediately before exposure to high fat food attenuates binge-like eating behavior in mice was found. This provides preclinical support for stimulating the NAc to block disordered and non-homeostatic feeding patterns. Such a therapeutic approach would require a closed-loop, or responsive neurostimulation device capable of anticipating and responding to a predictive signal of an upcoming binge. An increase in low frequency oscillatory power was recently discovered in the NAc during anticipation of a high fat meal that was used as a biomarker to trigger stimulation delivery in mice using a prototype of the RNS® System. This intervention blocked binge-like eating without adversely affecting homeostatic feeding, locomotor or social behavior in mice. These findings are expected to be translatable as functional neuroimaging studies in humans have demonstrated similar increases in NAc activity during anticipation of highly rewarding stimuli including palatable food. Moreover, using these very tasks during human NAc local field potential recordings (LFPs), temporally similar increases were found in low frequency power that were directly proportional to the magnitude of the reward. While these data are promising, further testing is needed to refine stimulatory parameters and test safety of intermittently stimulating the NAc, and confirm reliability and specificity of these LFP recordings in humans.

Results herein describe the potential of responsively stimulating the NAc for LOC eating using an FDA approved technology for another indication (the RNS® System) because: 1) responsive stimulation may be better tolerated than DBS due to less cumulative stimulation; 2) the neurostimulator is placed cranially without general anesthesia (unlike DBS); 3) preclinical studies revealed robust blockade of binge-like eating in mice with intermittently delivered NAc stimulation, but adverse effects on social behavior and a tolerance effect (i.e. loss of benefit) with open-loop DBS; 4) recent data in mice reveal an increase in low frequency oscillatory power in the NAc that is detected immediately prior to a binge, and effectively triggers stimulation-blockade of that binge; 5) similar NAc oscillatory changes were found in a human anticipating conventional rewards, and these changes in power were directly proportional to reward magnitude; 6) prior NAc region DBS studies typically utilized leads with large 4-mm spacing between contacts, precluding highly precise stimulation delivery to the NAc and its subregions. The planned depth leads are expected to have up to 3 separate contacts modulating the ventral, central, and dorsal regions of the NAc territory which should induce more predictable clinical effects.

The NAc region has been targeted previously for psychiatric disorders with deep brain stimulation (DBS), which is continuous and non-responsive (open-loop). Here, data are summarized from randomized controlled trials performed in treatment-refractory depression (TRD) and obsessive-compulsive disorder (OCD). Short-term follow-up during a blinded, 16-week treatment phase for TRD revealed improvement with active DBS, but this was not statistically significant. Adverse effects were tolerable and short-lived-in less than ⅓ of participants, worsening depression improved with parameter adjustment. Other less common adverse effects that resolved with re-programming included suicidal ideation, insomnia, irritability, disinhibition, hypomania, and mania. Mood elevation also occurred in a randomized trial of NAc DBS for severe OCD in 66% of participants, but in all cases mood elevation abated over time or responded to parameter adjustment. One hospitalization for OCD worsening occurred. Overall, the most concerning adverse effects in these representative studies were worsening depression and suicidal ideation, but these only occurred in the TRD study. In contrast, depression scores significantly improved for the entire OCD group during active DBS. OCD studies with long-term follow-up have reported durable levels of safety, and also significant relief, leading to a humanitarian device exemption by the Food and Drug Administration (FDA). This prior experience with NAc region DBS provides robust safety data that have guided planned intervention, as the adverse events reported have informed the design of an Early Feasibility Study (EFS).

Data described herein provides potential of responsively stimulating the NAc for LOC eating using an FDA-approved technology for another indication (the RNS© System) because: 1) responsive stimulation may be better tolerated than DBS due to less cumulative stimulation; 2) the neurostimulator is placed cranially without general anesthesia (unlike DBS); 3) preclinical studies revealed robust blockade of binge-like eating in mice with intermittently delivered NAc stimulation, but adverse effects on social behavior and a tolerance effect (i.e. loss of benefit) with open-loop DBS; 4) recent data in mice reveal an increase in low frequency oscillatory power in the NAc that is detected immediately prior to a binge, and effectively triggers stimulation-blockade of that binge; 5) similar NAc oscillatory changes were found in a human anticipating conventional rewards, and these changes in power were directly proportional to reward magnitude; 6) prior NAc region DBS studies typically utilized leads with large 4-mm spacing between contacts, precluding highly precise stimulation delivery to the NAc and its subregions. Planned depth leads are expected to have up to 3 separate contacts modulating the ventral, central, and dorsal regions of the NAc territory which should induce more predictable clinical effects.

Clinically meaningful device outcome measures. These include assessing battery life, biomarker detection sensitivity/specificity, triggering capability, data storage capacity, and stimulation tolerability. These features have been tested in the FDA-approved device in epilepsy patients in both pivotal and long-term trials, but first-in-human EFS will evaluate them in a novel indication. Feasibility of using the candidate local field potential (LFP) biomarker to program storage of LFP snapshots using the off-the-shelf RNS® System will be assessed. This biomarker has been identified in a preclinical model and a human anticipating financial rewards, and will be re-examined and refined in obese humans. Participants will be asked to signify onsets of LOC by swiping a magnet over the neurostimulator to store LFPs. Counts of magnet swipes (described herein) will also provide timestamps of LOC events in the ambulatory setting, as is routine when seizures are sensed by epilepsy patients. Concordance of magnet swiping as an event recorder (no stimulation) to other methods for logging LOC events, such as ecological momentary assessment (EMA) and a wrist-worn bite counter will be examined. Correspondence of these ambulatory assessments' impact on LFPs and the controlled laboratory evaluations will be reviewed. Subjects will be monitored for stimulation-induced adverse effects with strict go/no-go criteria. It is noted that there are no known efforts elsewhere examining responsive stimulation for LOC eating.

Minimally acceptable and ideal results. Minimally acceptable results include an adverse event rate consistent with DBS (though the overall rate is expected to be less) and the RNS® System for epilepsy. An eating disorder battery is utilized to assess adverse effects and non-futility. Futility is defined as no decrease in the #LOC episodes/week in at least 50% of subjects during the 6-month stimulation period following optimization in Stage 1 (or an increase in LOC episodes in no more than 50% of subjects). Effects of implantation and stimulating the NAc will be monitored. Feasibility of magnet swiping as an event recorder and the computer tasks and LOC lab as valuable correlates to ambulatory data is examined. Ideal outcomes include feasibility of implementing controlled and ambulatory assessments to refine the biomarker and safety/tolerability of NAc stimulation.

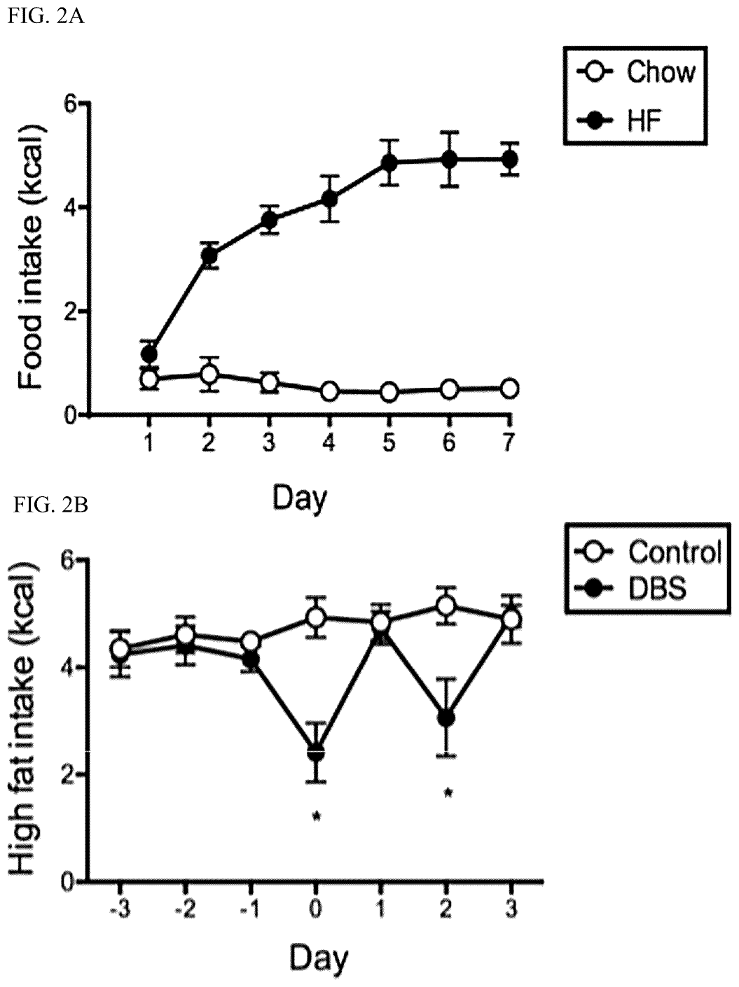

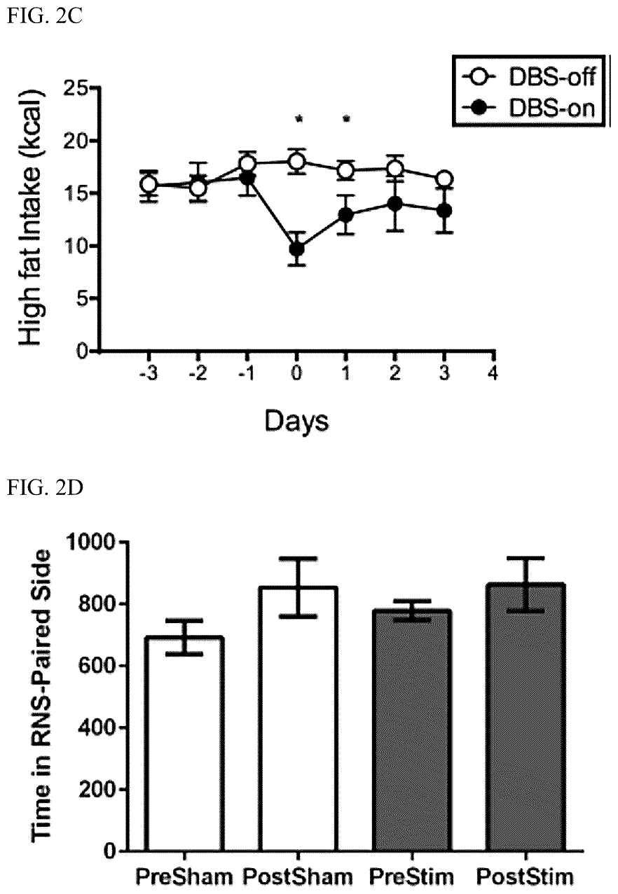

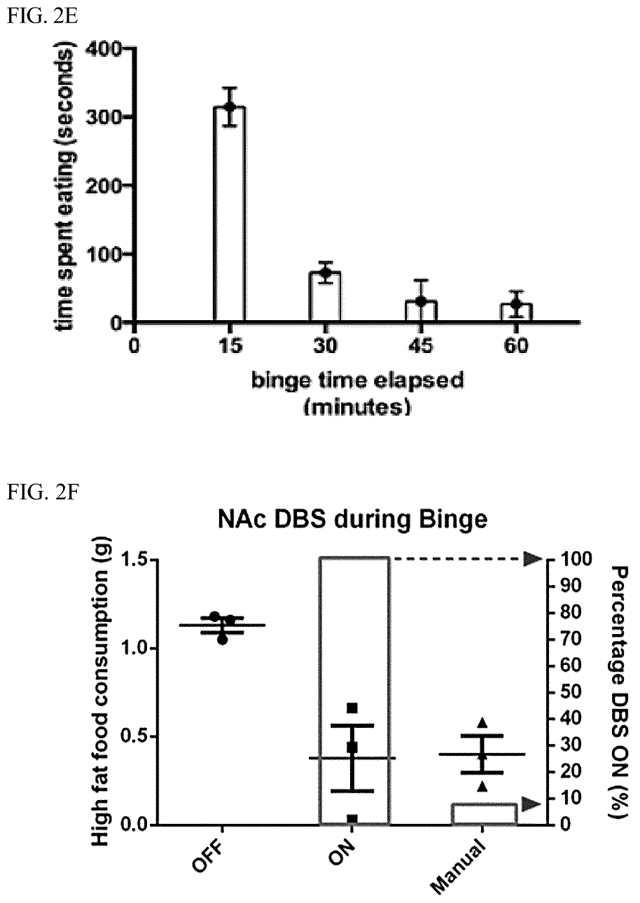

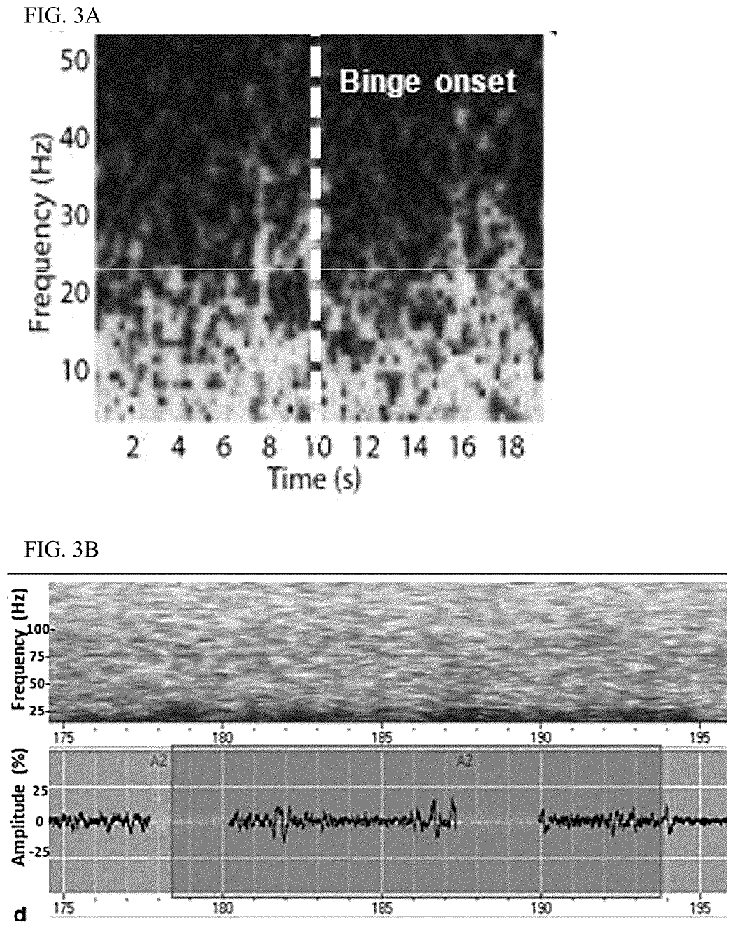

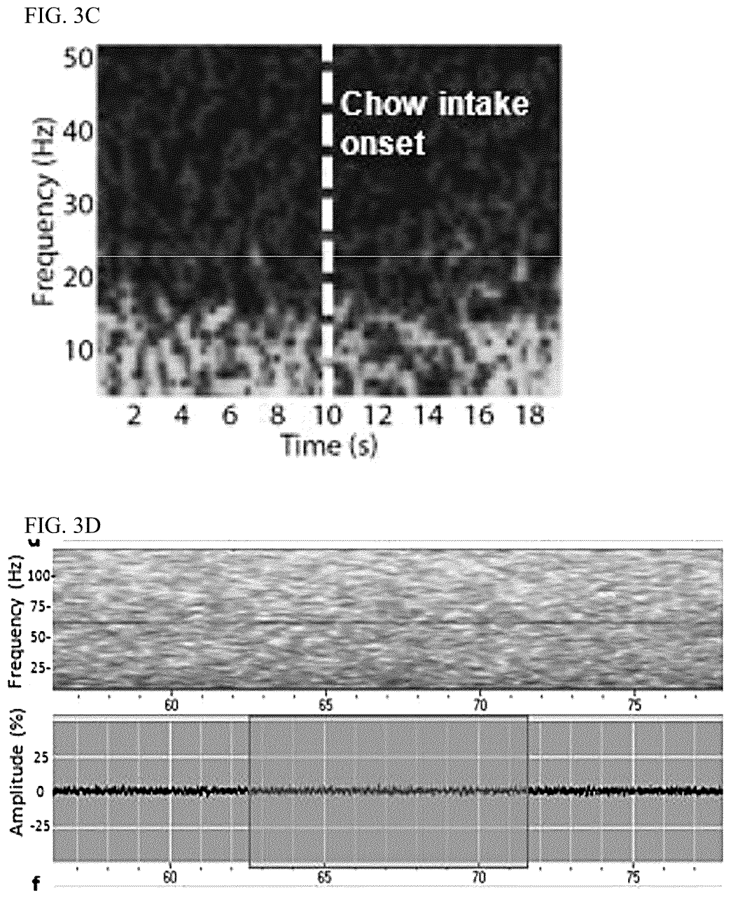

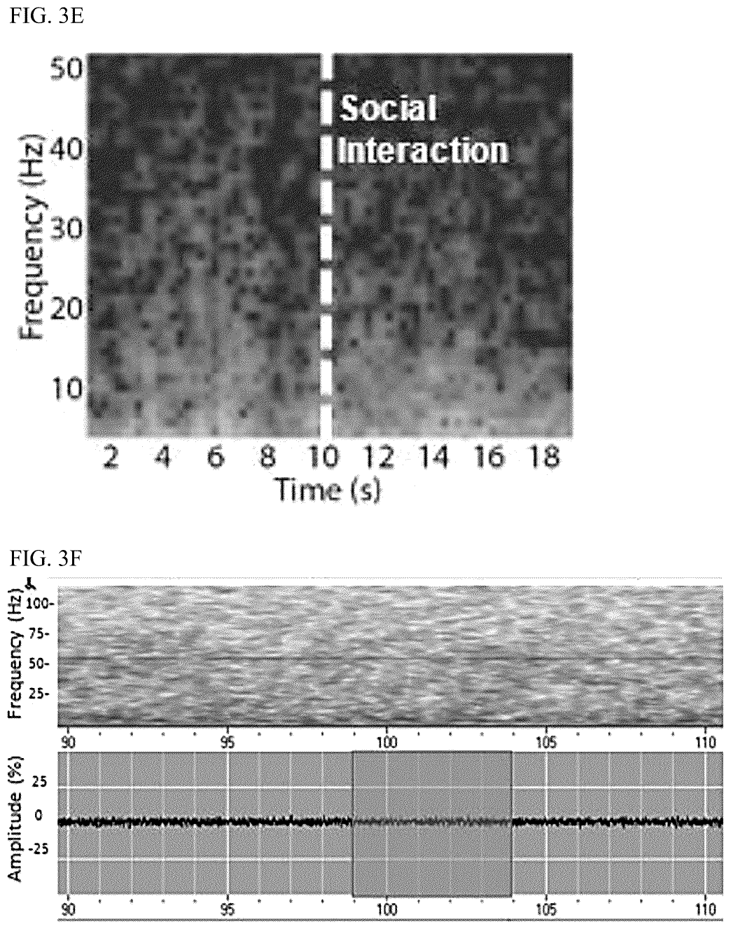

Intermittent NAc stimulation in mice (FIG. 2A). It was found that electrically stimulating the NAc (130 Hz, 60 us, 150 uA) immediately before and during a high-fat meal attenuates binge eating (FIG. 2B), and this effect exhibited dose response. Chronic studies in obese mice revealed reduced intake with continuous stimulation, but a tolerance effect was evident (FIG. 2C). NAc stimulation using these parameters did not induce a place preference, suggesting binge blockade did not involve reward substitution (FIG. 2D). Binge eating is episodic in mice much like LOC eating in humans (FIG. 2E). This episodic behavior, combined with an apparent tolerance effect to continuous stimulation supports stimulating only in the presence of a binge. Indeed, binge blockade was seen in preliminary studies with manually administering stimulation only during approach to high fat food with >90% decrease in the cumulative dose (i.e. percent time-on) (FIG. 2F). However, in order to translate this to humans, a device capable of anticipating an upcoming binge is needed. The closed-loop rig integrated a prototype NeuroPace device to test whether this already approved system could effectively detect and block binge eating in mice (FIGS. 3A and 3B).

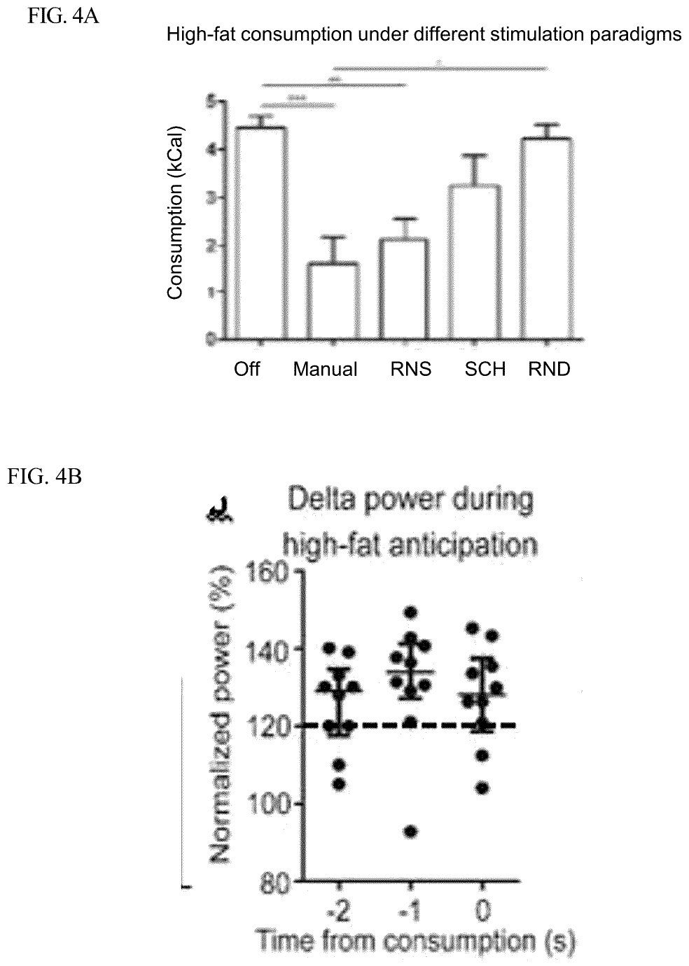

Rodent study with RNSprototype. Recording of real-time LFP activity from the mouse NAc using a RNS prototype (FIGS. 4A-4C). Increased power in low frequency oscillations (delta) prior to binge onset was detected. Delta's specificity was assessed by examining LFPs prior to chow intake and social interaction, which were not associated with increased delta power. Multiple stimulation protocols were assessed, including RNS optimized by delta power (RNS), scheduled (Sch), and random stimulation delivery (Rnd) (FIG. 4A). Delta peaked during a 2 s interval immediately prior to a binge. Any power crossing a 20% threshold increase was used to trigger RNS (FIG. 4B). RNS did not impact social interaction (FIG. 4C).

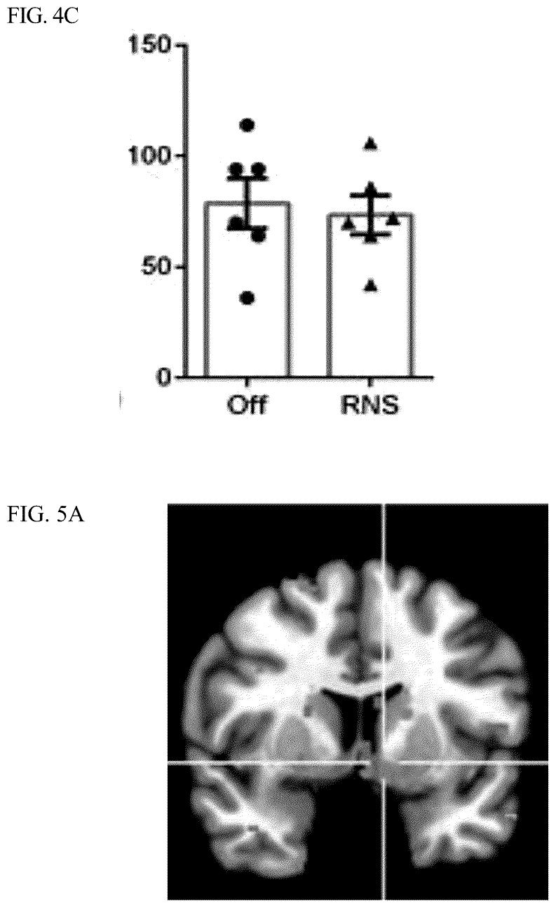

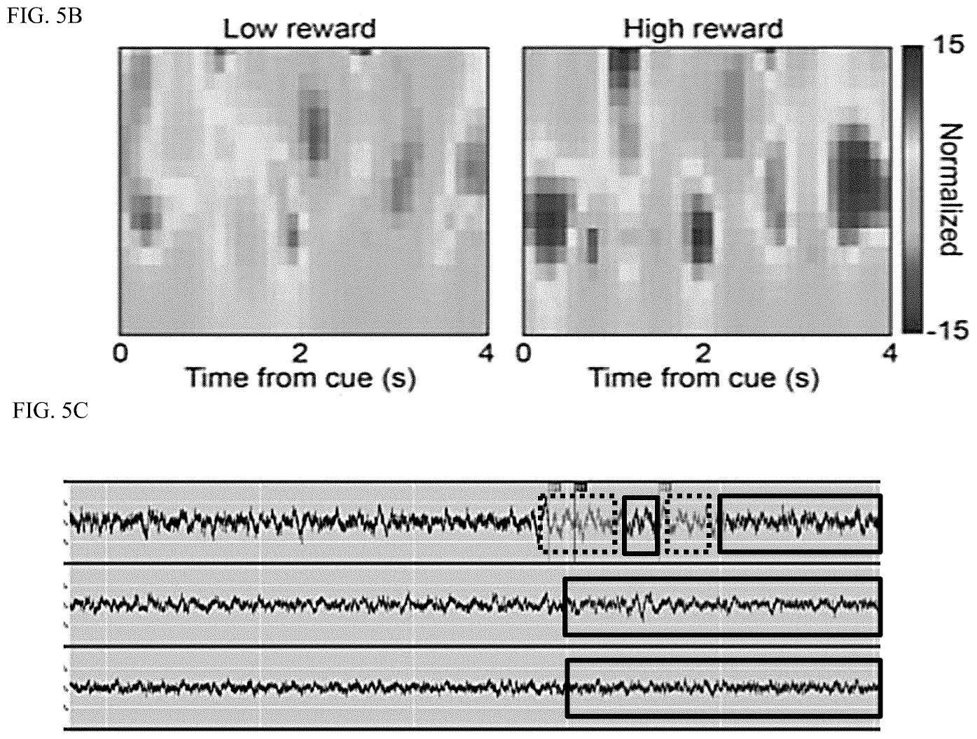

Human NAc LFP recordings. Complementing these data are intraoperative recordings from the human NAc. In an IRB-approved study, an OCD patient partook in a financial task that was confirmed to recruit NAc activity with fMRI (FIG. 5A). This study revealed changes in delta during anticipation of gain, which like BOLD signal were proportional to the magnitude of reward gain (FIG. 5B). This BOLD signal in the human NAc has been reported across multiple studies to be robust during reward anticipation in humans, and its intensity appears specific to highly rewarding stimuli, much like what was seen in binge eating mice. It was confirmed that existing detection algorithms could sense delta using a benchtop RNS (FIG. 5C), suggesting that LFPs from the mouse NAc can reflect the human NAc despite different electrodes, impedances, and sensing hardware. Moreover, changes in delta proportional to reward magnitude suggest potential for behavioral specificity of RNS for LOC eating. Further work is needed to assess biomarker specificity, reproducibility, and RNS detection algorithms.

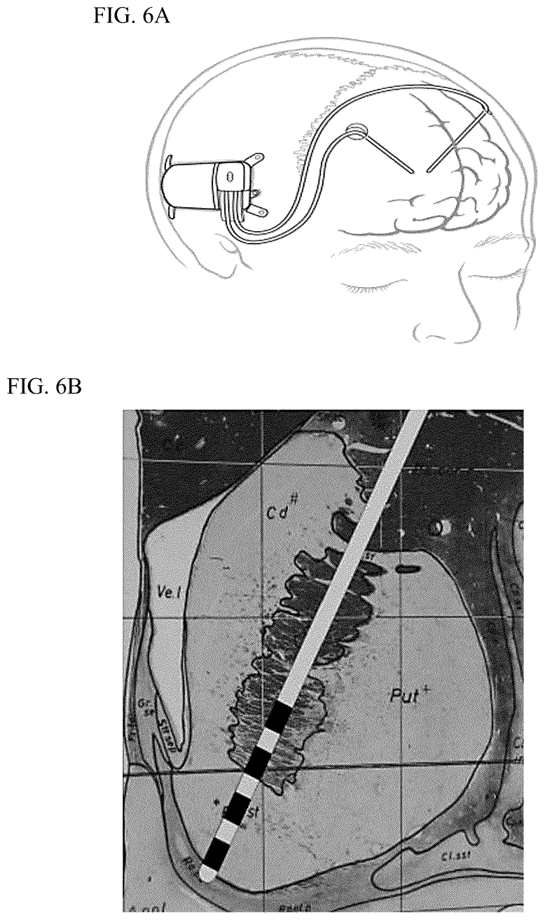

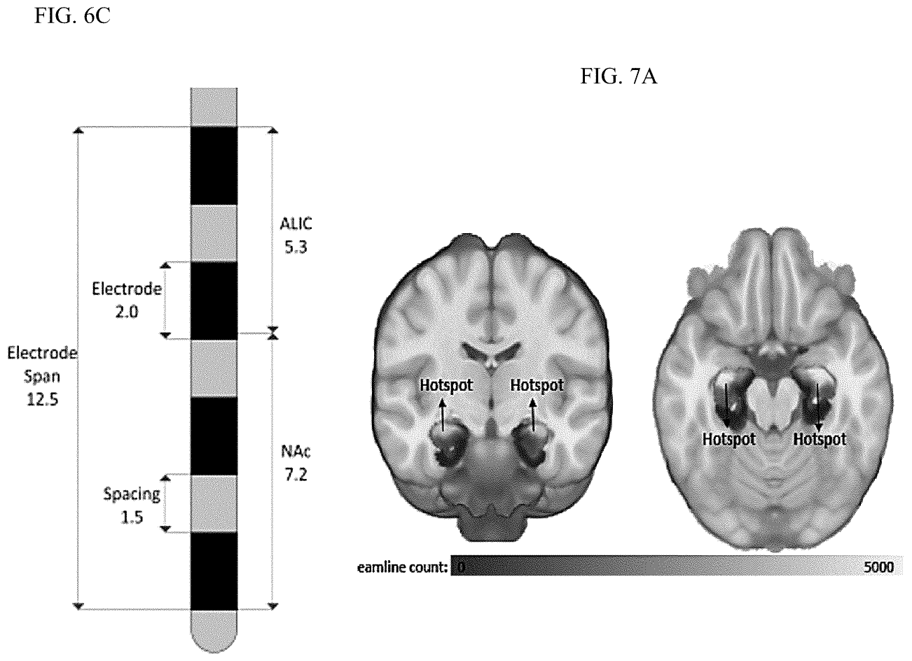

The RNS System. This is the first FDA-approved, intracranial, closed-loop system, and includes a cranially-implanted responsive neurostimulator connected to 2 leads, a physician programmer, patient remote monitor, and an internet-based data repository for physicians to review stored LFPs. The proposed system here includes approved depth leads targeted to the NAc (FIGS. 6A-6C). The depths (DL-330-3.5) have 4 electrodes with 2 mm spacing. Using this system for the proposed studies will leverage: (1) FDA-approved hardware; (2) prior clinical safety data; (3) chronic implantation; (4) responsive stimulation capabilities; (5) ability to collect a rich LFP dataset; and (6) a proven user interface. Should a wireless software update be needed based on Stage 1 of the EFS, a research RNS (rRNS) System will incorporate new software features only.

Clinical Safety Experience. The safety profile of the RNS® System is complemented by safety/efficacy data on stimulating the NAc with DBS for OCD and TRD. Moreover, OCD studies have demonstrated that significant improvements can be predicted by a positive affect induced during monopolar assessments. The most common adverse effect is transient hypomania at high voltages. Thus, current amplitude will be kept low, starting at 0.5 mA, and psychiatrists with expertise in NAc DBS will closely monitor subjects during stimulation phases. Given that the NAc is only intermittently stimulated, it is expected that the overall “dose” (i.e. time-on) is significantly less than DBS (FIG. 2F). In fact, even using a duty cycle (1 min on, 5 mins off paradigm) leads to 4 hrs/day of stimulation as was attempted in the SANTE DBS epilepsy trial, which is far higher than the average time-on in NeuroPace's pivotal trial of about 5 mins/day total (delivered intermittently throughout the day in 100 ms bursts only when electrographic seizures are detected). Last, as NAc stimulation has never been performed in treatment refractory obese patients with LOC eating, a comprehensive eating disorder and bariatric monitoring approach is planned to enhance objectivity and complement surveillance visits performed by psychiatrists.

Real-Time Continuous LFPs with RNS® System During controlled assessments, NeuroPace accessories will be used to record real-time continuous LFPs while patients participate in videoed behavioral lab studies (e.g. Milkshake Paradigm, LOC Lab Study). This multimodal approach was of interest to the FDA during a pre-IDE submission meeting as they are expected to facilitate off-line LFP analyses, optimize specificity for LOC eating, control for normal meals and natural rewards, and activate detectors for LOC in the ambulatory setting to ensure storage of LOC bouts automatically. Subjects will be able to magnet swipe to timestamp LOC sensations as well.

Chronic Implantation. One approach used to develop neurotechnologies has been initial testing in the subacute setting. There are concerns about generalizability of subacute responses to stimulation. During the NeuroPace pivotal trial, median seizure reduction increased from 38% at 3 months to 66% at year 3. 6, 47 Similar improvements over time have been observed for other neuromodulatory therapies. There is also a subacute implant effect causing seizure reduction, as has been noted in movement disorder, OCD and depression patients in which temporary relief is noted post-implantation. Thus, stimulation effects are expected to be more stable 4 months after implantation, when recording labs and the stimulation phase is initiated.

Responsive stimulation. The RNS® System has also been used in an ongoing investigator-initiated trial to examine other episodic disorders, such as Tourette Syndrome. Stimulation resulted in tic reduction, and LFP recordings revealed a potential predictor of tic occurrence. LOC eating is another episodic disorder, and the RNS® System's success will help identifying biomarkers of epilepsy and Tourette to define for the first time an LFP signal that predicts LOC and effectively triggers stimulation to block a subsequent binge.

Stage 1 of EFS (n=2) and as needed software development. Stage 1 of the EFS will be conducted in a small, initial cohort (n=2) following Stanford TRB approval. This stage will test the safety and feasibility of implanting the existing RNS System into the human NAc, its ability to identify an electrographic biomarker of LOC eating, and the tolerability/safety of intermittent NAc stimulation. Pending confirmation that the existing capabilities of the system are sufficient to identify an indication-specific biomarker, Stage 2 of the EFS will be performed for the remaining 4 implants. This staged approach was the agreed upon EFS design at a FDA PreSubmission meeting.

Implant Safety and Feasibility. The initial two subjects will be implanted (staggered by 1 month) with the neurostimulator and leads according to the procedure described below (Table 1). Clinical Protocol Synopsis details the EFS. Only a brief overview is provided here. All subjects will undergo psychiatric interviews, involving eating disorder and bariatric assessments, as well as a diagnostic evaluation by a psychiatrist with expertise in NAc stimulation. After surgery, subjects will be seen at least monthly by the contact PI, a psychiatrist, a clinical research coordinator, and a NeuroPace field engineer, where neurological/physical and general psychiatric exam, RNS functioning, adverse events, an eating disorder battery, body weight, lab tests, as well as EMA, bite-counter, and LFP data will be assessed. This phase of the EFS is designed so that a substantial recording optimization phase with controlled and ambulatory assessments precedes intermittent stimulation testing.

TABLE 1
Timeline of events.
No Device Changes

| | Timeline | | | | | | | | | |
|---|---|---|---|---|---|---|---|---|---|---|
| | Year 1 | | | | Year 2 | | | | Year 3 | |
| Calendar Quarter | 0.418 | 0.119 | 0.219 | 0.319 | 0.419 | 0.120 | 0.220 | 0.320 | 0.420 | 0.121 |

Regulatory

| | |
|---|---|
| IRB Submission | X |
| IRB Approval | X |

Aim 1: Clinical - Initial 2 Subjects (1-2)

| | | | | |
|---|---|---|---|---|
| Implant | X | X | X | X |
| Recording Only | X | X | X | X |
| Interim Report on | X | X | X | X |
| biomarker | | | | |
| Stimulation | X | X | X | X |
| Interim Report | X | X | X | X |

Aim 2: Clinical - Remaining Subjects (3-6)

| Implant Subjects | | | | | X | | | X | X | X |
|---|---|---|---|---|---|---|---|---|---|---|
| Recording Only | | | | | X | | | X | X | X |
| Stimulation | | | | | X | | | X | X | X |
| Interim Report | | | | | X | | | X | X | X |
| Implant Subjects | | | | | X | | | X | X | X |
| Recording Only | | | | | X | | | X | X | X |
| Stimulation | | | | | X | | | X | X | X |
| Final Report | | | | | X | | | X | X | X |
| | Timeline | | | | | | | | | |
| | Year 3 | | Year 4 | | | | Year 5 | | | |
| Calendar Quarter | 0.221 | 0.321 | 0.421 | 0.122 | 0.222 | 0.322 | 0.422 | 0.123 | 0.223 | 0.323 |

Regulatory

IRB Submission
IRB Approval

Aim 1: Clinical - Initial 2 Subjects (1-2)

| | | | |
|---|---|---|---|
| Implant | X | X | X |
| Recording Only | X | X | X |
| Interim Report on | X | X | X |
| biomarker | | | |
| Stimulation | X | X | X |
| Interim Report | X | X | X |

Aim 2: Clinical - Remaining Subjects (3-6)

| | | | | |
|---|---|---|---|---|
| Implant Subjects | X | X | X | |
| Recording Only | X | X | X | |
| Stimulation | X | X | X | |
| Interim Report | X | X | X | |
| Implant Subjects | X | X | X | |
| Recording Only | X | X | X | |
| Stimulation | X | X | X | |
| Final Report | X | X | X | X |

Data Analysis. Results will be described using standard summary statistics, evaluating outcomes at each visit and assessing changes from baseline. Standard summary statistics will also be generated across the 6 subjects. This study will be used to inform future power calculations for a formal evaluation of safety and efficacy, but the primary intention here is feasibility and non-futility testing, as no prior data has ever been collected on the use of this device for this indication. Descriptive statistics will be provided to evaluate changes within subject for LOC and mood assessments taken before and after an eating episode. Results obtained from multimodal ambulatory assessments of LOC episodes will be assessed for congruence and evaluated graphically. Spearman and Pearson correlation coefficients will be calculated. Feasibility of calculating the performance of LFPs as predictors of LOC episodes will be examined using each of these 3 separate approaches. Diagnostic accuracy will be calculated for each method.

Surgical Procedure. A standard frameless stereotactic approach will be used to target the NAc. Each depth lead has 4 independently programmable cylindrical electrode contacts. Per a targeting protocol for OCD surgery, the distal-most contact (0) is expected to be in ventral NAc. Given the distance between contacts on this depth lead, contacts 1 and 2 will be in central and dorsal NAc and partially the anterior commissure. The dorsal-most contact (3) will be in the anterior limb of the internal capsule. Sedation is administered, and the head is prepped and draped. Incisions are made, and disposable stereotactic navigation hardware is placed. The outer cannula is placed into the brain 20 mm above the target on the first side. A microelectrode is advanced stepwise, continuously recording single- and multi-unit activity for 1 min every 1 mm to physiologically define the NAc region. This is repeated contralaterally. Once the depth leads are implanted, intraoperative imaging is obtained to confirm accuracy (<2 mm radial error). Leads are then secured in place, and a small right parietal craniotomy (2×4 cm) is made for the neurostimulator. The depth leads are connected to the device, and an impedance check and real-time LFPs are performed to confirm a functioning system.

Monopolar Assessment. Immediately prior to initiating stimulation in the EFS, a monopolar stimulation assessment will be performed. Stimulation is initiated and titrated using current intensity ranges from OCD protocol (initial parameters: 130 Hz, 90 μs, and 0.5 mA). The anticipated and desired responses are elevations in mood, and facial expressiveness. Patients who will be blinded to test conditions are asked to report their mood, anxiety, and alertness verbally using 10-point scales. Stimulation is tested for approximately 2 min with the subjects blinded, interspersed with periods of no stimulation. Initially, the current amplitude will be set at 0.5 mA less than the lowest amplitude needed to induce a positive affective response at a single contact (to be assessed up to 4 mA). Stimulation will be initiated unilaterally.

After this monopolar assessment, initial 2 subjects will enter a 6-month recording period, during which continuous, real-time NAc LFPs will be performed in the ambulatory setting using multiple modalities to target timestamped LFP for off-line analyses and in the LOC laboratory with video surveillance. Congruence of the controlled and ambulatory assessments of LOC eating and LFPs will be measured. During this phase, recording/detection parameters will be set based on prior experience with RNS and signals obtained in prior mouse and human studies. Indeed, these algorithms have already exhibited effective detection of the candidate LFP biomarker, suggesting promise for using the unmodified off-the-shelf system.

Ambulatory assessments: Multiple 1-3 min snapshots of LFP activity will be recorded daily; recordings will be triggered by detection of the candidate LFP biomarker (FIGS. 3A-3F and 5A-5C) to be refined during this recording phase based on individual data. Snapshots of LFP recordings will also be stored in response to magnet swipes and at scheduled times-of-day. These will allow confirmation that the automatic detectors are optimized for the biomarker. Snapshots EMA and bite-counter timestamps will also approximate times-of-day of LOC and normal meals as well as periods without eating to direct LFP analyses for an indication-specific biomarker. The subjects magnet swipe when LOC is sensed, as is routine for EMA logging: Fidelity of magnet swiping will be assessed by reviewing EMA timestamps. The bite-counter may also provide precise timestamps of meal onsets. Such multimodal timestamps will facilitate a targeted offline LFP analysis.

Controlled assessments: Laptop tasks (Milkshake Paradigm, Monetary Incentive Delay-described in Clinical Protocol Synopsis) that evoke NAc BOLD signal will be used to assay NAc LFPs (see Clinical Protocol Synopsis). LFPs will be streamed to the programmer for offline analyses. Laptop commands will be sent to the programmer and video acquisition computer to introduce markers for synchronization. These computer tasks are intended to induce LFP changes during anticipation phases of nonfood and food reward receipt, such that it can assess specificity and the biological redundancy of the candidate biomarker in a controlled setting parallel to offline analyses of ambulatory data. Behavioral laboratory assessment of LOC eating (validated LOC eating lab with multi-item buffet, see Clinical Protocol Synopsis for details) with real-time recording and LFP streaming. Commands will be sent from the video acquisition computer to the programmer to initiate LFP recording and to introduce timestamps in the LFP records. Commands will trigger LED lights on the RNS accessories that are included in the video to allow for synchronization. These studies will optimize detection settings for the candidate biomarker as is typically done for epilepsy with seizure induction in epilepsy monitoring units (FIGS. 3A-3F and 5A-5C).

Biomarker identification. LFP snapshots will be recorded and downloaded daily from the device by each subject and uploaded to a secure web-based data management system as is routine for epilepsy patients. Clustering algorithms may be used to differentiate LFP features associated with LOC eating. Analyses will update as new data are uploaded providing near real-time biomarker identification. A similar analysis has been successfully implemented by NeuroPace for epilepsy patients. Identical methods will be used to identify biomarkers in the continuous real-time LFP data collected during controlled assessments. The LOC multi-item buffet lab is intended to induce LOC in a controlled, videoed setting much like seizures are induced in epilepsy monitoring units in patients undergoing synchronized electrographic analyses. The laptop-based tasks will be utilized to assess specificity of the candidate biomarker for an anticipated palatable meal preceded by LOC. Tasks known to recruit NAc activity will be utilized to probe this region and assess electrographic anticipatory responses to conventional rewards like money. LFP analyses informed by LOC eating logs will be conducted in all stages of the clinical study. LFP activity is assessed between stimulations. Over the course of the day, the total amount of stimulation is anticipated to be less than 5 mins based on mouse and clinical epilepsy experience. During stimulation, the RNS settings will be adjusted to minimize sample loss during stimulation allowing for near concurrent sense and stimulation. This capability for simultaneous sensing and stimulation may also provide a physiologic read-out for titrating stimulation parameters in the clinic using laptop-based tasks.

Safety and Tolerability. To enhance the objectiveness of behavioral ratings and measuring side effects of stimulation initially, a randomized blinded staggered-onset design adapted from prior studies of DBS will be performed for psychiatric disease. At the planned stimulation start date, subjects will be randomized to either RNS or sham. Half of the patients will have stimulation turned on, and the other half will have stimulation initiated 2 months later. The Maintenance of the Blind Plan will involve blinding all subjects and the study team members to the assigned grouping until the second cohort has had at least 2 months of follow-up to assess at least initial safety and placebo effects. All programming will be performed by an unblinded physician with expertise in NAc stimulation. In the event of a serious side effect, the involved subject is unblinded.

Stimulation will be initiated in the ambulatory setting for 1-week duration, followed by a 1-week evaluation period by the study's interventional psychiatrist and PI's. If there are no serious adverse effects, stimulation will be re-initiated for 4-weeks, followed by another 1-week duration. If there are no serious adverse effects, stimulation will be re-initiated for the duration of the assessment. Safety assessments include treatment-emergent adverse event documentation, weekly-monthly monitoring visits, psychiatric assessments, the Columbia-Suicide Severity Rating Scale, vital signs, body weight, labs, and overall nutrition throughout the study. Weight is recorded using a calibrated scale in shoeless participants, rounded to the nearest 0.5 pounds, and converted to kilograms. Telemetry and CPAP will be available as needed for all subjects in house. In addition to weekly-monthly visits with the contact PI and psychiatrist, a postoperative clinical visit in the bariatric clinic is planned at 3 months and 1 year postoperatively. Labs and an electrocardiogram will be obtained, and vital signs, urinalysis, and serum levels of iron, B12, B1, calcium, acid-base disturbances, amylase, glucose tolerance testing, and a lipid panel will be evaluated. Three additional clinical evaluations will be conducted during the stimulation phases to support any untoward side effects, as well as neuropsychology and nutrition visits for unanticipated effects, including worsening of LOC or even overly restrictive eating consistent with post-surgical eating avoidance disorder. Routine eating disorder assessments ensure these are detected early.

LFP-Responsive Stimulation (12 months). If a biomarker has been identified, responsive stimulation will be initiated randomly in half the sample as described above (see Table 1). The initial stimulation parameters will be informed as described in Monopolar Assessment. Laptop tasks and the LOC eating lab will be used to assay detector and stimulation specificity as well as safety prior to initiating stimulation in the ambulatory setting. Stimulation and detection settings may be adjusted at weekly-monthly clinic visits based on clinical judgment, LOC logs, eating disorder questionnaires, and adverse effects. The contralateral lead's optimal contact defined during the monopolar assessment will be activated after the first week of stimulation, as is a standard protocol for OCD. Stimulation will be delivered only when a biomarker is detected, and magnet swipe, EMA and bite-counting logs will continue.

### Example 2: Multimodal Elucidation of the Feeding Circuit Involving the Human Hippocampus

State-of-the-art techniques allow modulations of the feeding circuit to provoke predictable changes in feeding behavior in animal models. Food cues activate neuronal populations in the hippocampal area, which receives orexigenic projections from the lateral hypothalamus (LH) feeding circuit. While direct modulation of LH is challenging in humans, the hippocampal area may be an alternative area to modulate the feeding circuit. Here, a multimodal approach is used to characterize the human hippocampal involvement in the feeding circuitry. Probabilistic tractography-defined connections between the hippocampal area and LH converged in a dorsolateral hippocampal subregion (dlHPC). Resting-state functional connectivity and probabilistic tractography between the dlHPC and the LH revealed differences in this circuit between obese binge eaters and healthy controls. Furthermore, for the first time, LH projections were identified in feeding within the dlHPC using brain-clearing 3D histology. Finally, in a group of subjects implanted with depth electrodes, consistent increase in low-frequency responses during anticipation of a highly palatable compared to a tasteless solution was observed in the dlHPC.

Obesity represents one of the most challenging public health crises worldwide, and current treatment options have limited long-term efficacy. Dysfunctional control of feeding behavior is a primary cause of this limited treatment efficacy in subjects with refractory obesity.

Food cues activate hippocampal neuronal populations that regulate food intake and encode food-place memory. Hippocampal function play a role the decision making involved in the pre-consummatory component of feeding behavior, what has been hypothesized to be related to orexigenic projections it receives from the LH. Targeting the hippocampal area to indirectly modulate this circuit may have extraordinary potential to change the course of the binge eating and obesity public health crisis.

The lateral hypothalamus (LH) has been, for decades, implicated in controlling feeding and consummatory behavior. Human investigations with functional MRI (fMRI) have revealed activation of the hypothalamus and a related feeding circuitry in response to high-fat food cues, which was significantly greater prior to high-fat food ingestion. This is likely to be in part due to orexigenic neuropeptides, such as melanin-concentrating hormone (MCH), which is produced in the LH and distributed to several brain regions. Preclinical studies have demonstrated that stimulation and inhibition of LH signaling respectively increases and suppresses food intake and consummatory reward. Hence, the modulation of this circuit may hold relevance for the treatment of obesity and binge eating. Invasive clinical trials for refractory morbid obesity have found directly modulating the LH extremely challenging and imprecise due to its functionally dense organization, which is incompatible with current clinically available hardware. Alternatively, targeting long-distance projections has proven to be viable for modulation of brain circuits.

Diffusion (dMRI) and functional MRI (fMRI) are the standard methods for indirectly rendering structural and functional brain circuits for targeting fiber pathways in clinical research settings. High-resolution dMRI tractography may allow in-vivo localization of where projections to and from the lateral hypothalamic feeding circuitry converge within the hippocampal area. Combined, these methods can shed light on the role clinical role of this LH-hippocampal circuit in the obesity and binge eating. One major limitation of this method, however, is that it is prone to false positives and negatives, once it relies on geometric patterns of water diffusion to indirectly infer on the orientation of axonal bundles and render fiber projections. This limitation is addressed by supplementing the approach with post-mortem brain clearing 3D histology. This modality has emerged as a possible key for reliable application of dMRI by allowing direct visualization and characterization of neuronal projections within circuit-based hotspots. These methods can also inform targeting of invasive electrophysiology investigation in human subjects undergoing stereoencephalography (stereo-EEG), which represent a unique opportunity to examine anatomically distinct brain responses during the pre-consummatory phase (anticipation) of feeding behavior using controlled feeding-related assays.

To investigate the role of the hippocampus in the feeding circuitry and to define a potential target within the human hippocampal area to modulate feeding behavior, the followings are combined: (1) ultra-high resolution probabilistic tractography from 178 healthy subjects from the Human Connectome Project (HCP); (2) clinical imaging data, including resting-state functional connectivity (rsFC) and probabilistic tractography connectivity indices (tractography-CI) from a distinct cohort of 34 subjects with at least one weekly binge eating episode and 24 controls; (3) iDISCO brain-clearing 3D histology in a post-mortem sample of the left human hippocampal area; and (4) intracranial electrophysiology from 8 subjects implanted with hippocampal depth electrodes during anticipation of palatable food in a controlled task.

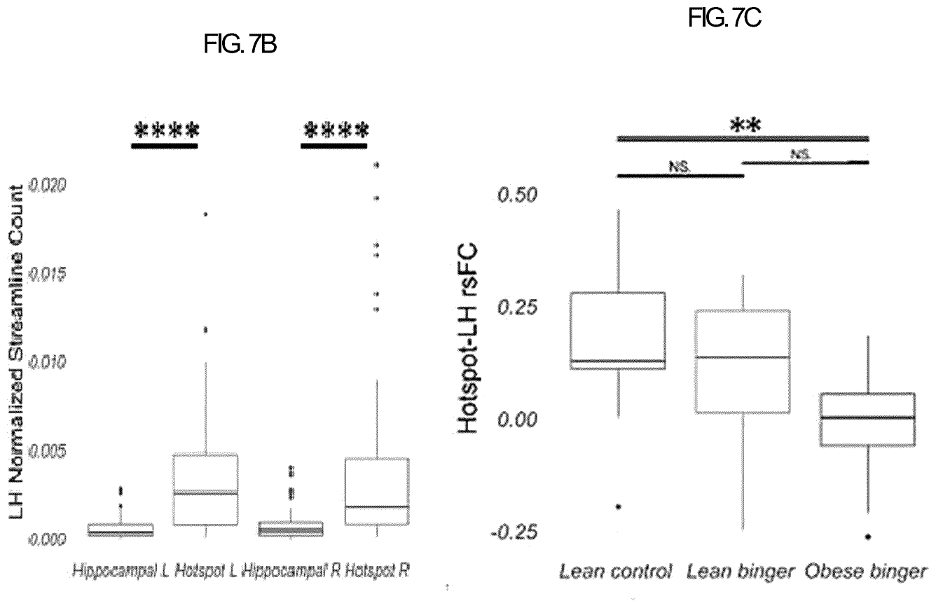

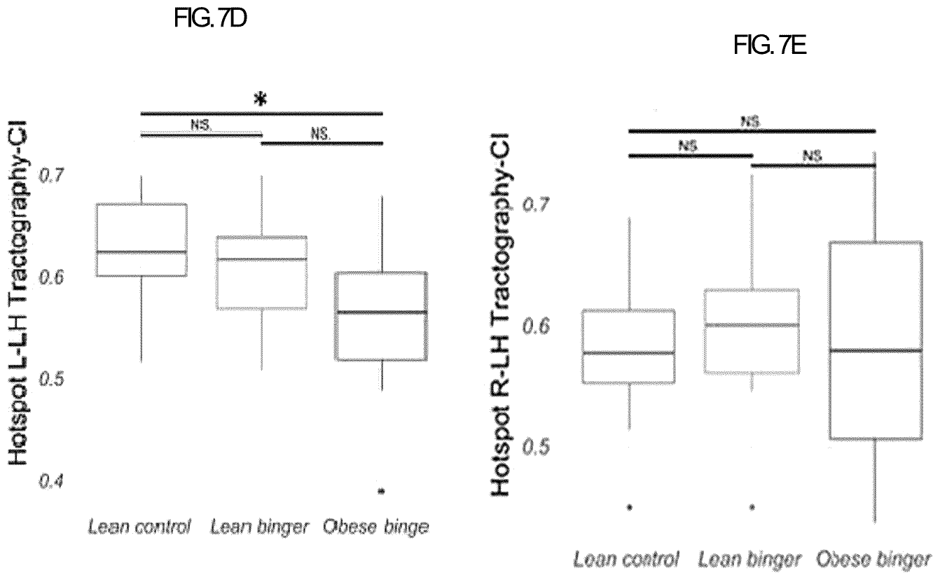

Probabilistic tractography in HCP subjects' high-resolution data revealed that tractography-defined LH connections (streamlines) converge in a dorsolateral hippocampal subregion (dlHPC) (FIG. 7A). Indeed, a similar analysis performed in clinical data confirmed a significantly higher normalized streamline count (i.e. tractography-defined connections) between the LH and the left (p<0.0001) and right (p<0.0001) dlHPC as compared to the whole hippocampal area, indicating a higher relative probability for the algorithm to track streamlines to the LH from dlHPC voxels as compared to the all hippocampal voxels (FIG. 7B).

To investigate the clinical relevance of the dlHPC, its functional and structural relationship with the LH specifically in obese (i.e. BMI>30 m²/kg, n=12) and lean (i.e. body mass index, BMI <25 m²/kg, n=17) binge eaters (i.e. more than 1 weekly episode of binge eating) were assessed and compared to healthy controls (i.e. lean subjects with less than 1 weekly episode of binge eating, n=16). Outliers' measurements that were outside the 0.05 and 0.95 percentiles were removed, assuming a normal distribution of the data (confirmed with Shapiro-Wilk normality test, p>0.05). Interestingly, rsFC between the dlHPC and LH was significantly different between obese binge eaters, lean binge eaters and lean controls (Kruskal-Wallis chi-squared (H)=8.3919, df=2, p=0.015) (FIG. 7C). Post hoc testing revealed significantly decreased rsFC in obese binge eaters compared to lean controls and the (p=0.009, adjusted for false discovery rate (FDR) of multiple comparisons) and marginally significant decreased rsFC in obese compared to lean binge eaters (p=0.054, FDR-adjusted). Tractography-CI between the dlHPC and the LH was significantly different between groups for the left (F=3.418, p=0.043; Tukey's post hoc test: lean control vs. obese binger, p=0.034, revealing decreased tractography-CI in obese bingers) but not right (F=0.385, p=0.683) hemispheres (FIGS. 7D and 7E).

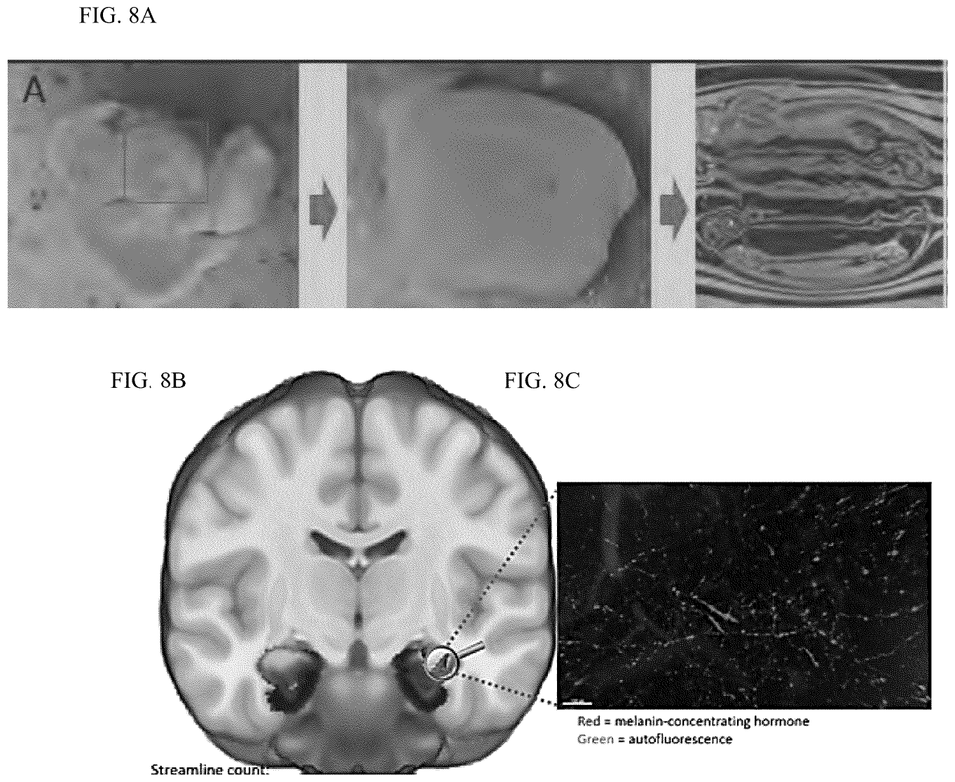

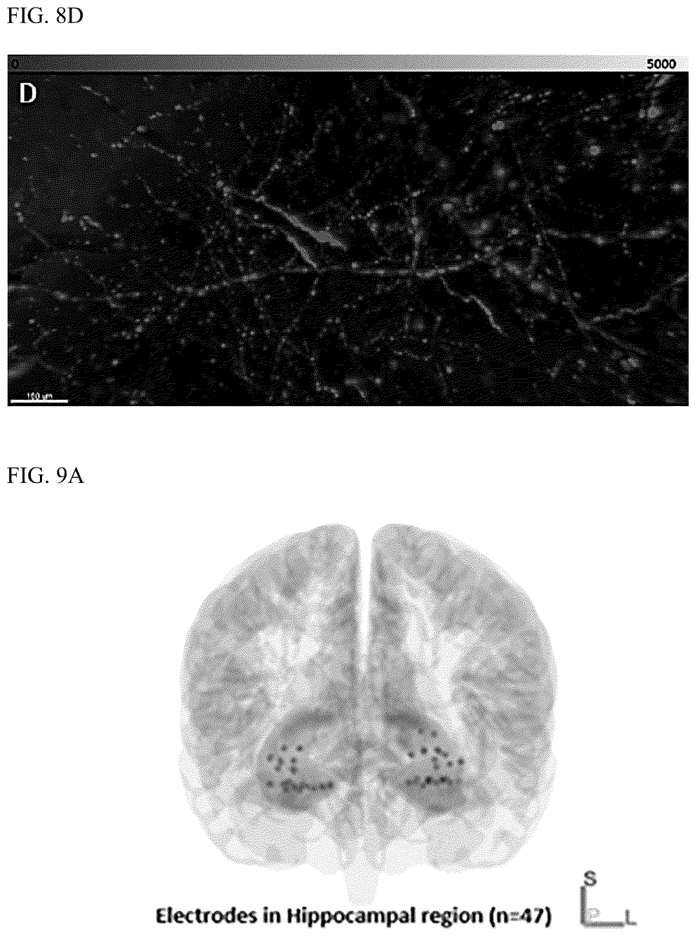

To further characterize the uncovered dlHPC, a post-mortem sample of the hippocampal area (including human entorhinal cortex and hippocampus) was obtained and a smaller subsection was selected for the immunolabeling-enabled 3D imaging of solvent cleared organs (iDISCO) procedure (FIG. 8A). This 3D histological brain-clearing method was used to identify the presence of lateral hypothalamic projections containing MCH within the human dlHPC. After validating the anti-MCH antibody for the iDISCO protocol, it was utilized to visualize LH projections in cleared hippocampal tissue. The MCH-stained sample was then overlaid to the high-resolution tractography-based MRI atlas derived from the dMRI data analysis defining hippocampal subregions with higher streamline probability to the LH (FIG. 8B). Finally, it was found that the location of higher number of streamlines between these two structures correspond to the subregions where MCH+ orexigenic projections could be visualized using the 3D brain-cleared histology (FIGS. 8C and 8D). These findings further corroborate the LH MCH-connectivity in the human LH-hippocampal circuit involved in obesity and binge eating.

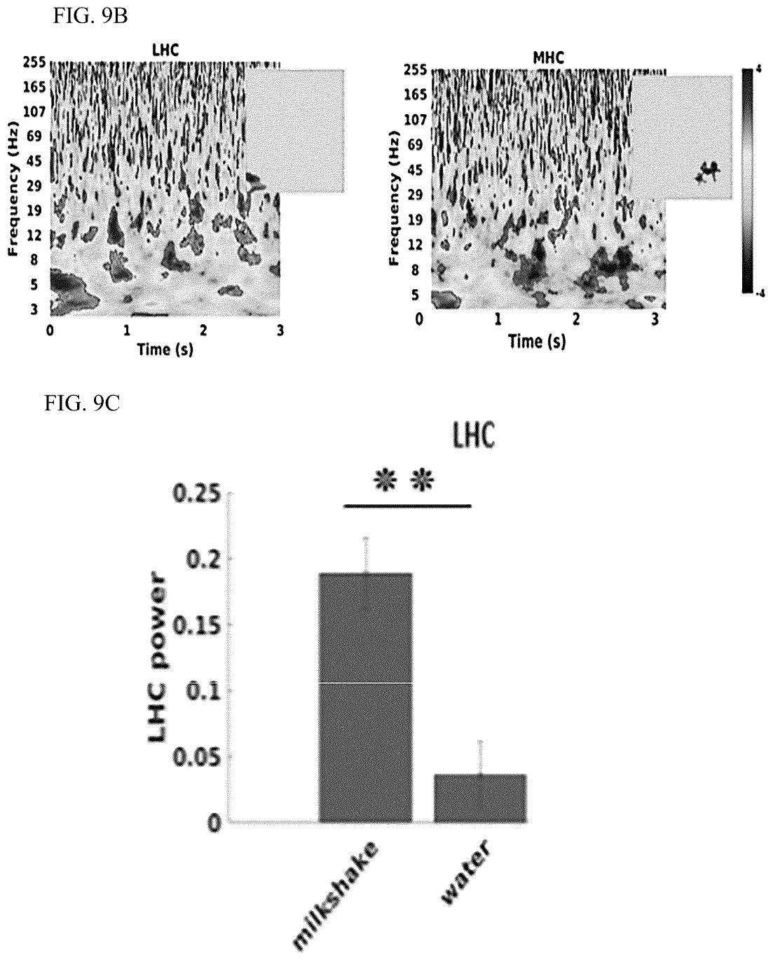

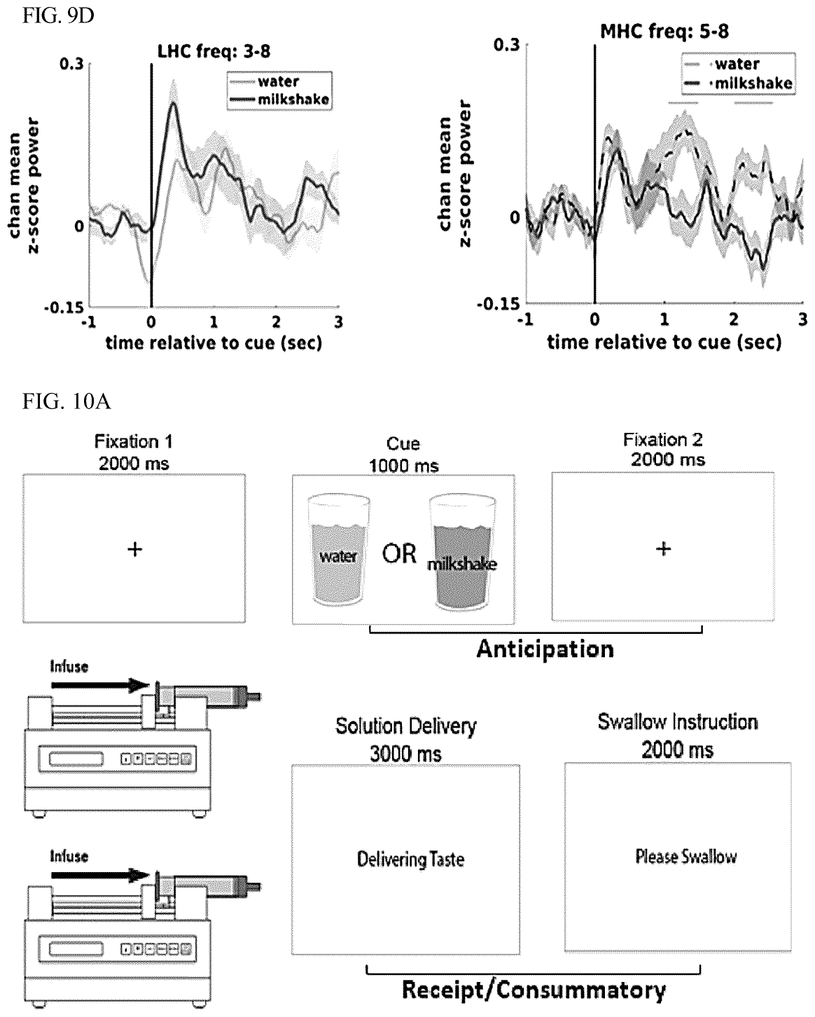

After identification of structural and resting-state functional links between the LH and the dlHPC, it was next investigated whether the dlHPC electrophysiological profile is differentially modulated with feeding behavior. More specifically, the tested hypothesis is that hippocampal hotspot spectral dynamics will differ between anticipation of palatable and neutral items. To do this, local field potential was recorded from electrodes (n=46) in the human hippocampus of subjects undergoing stereo-EEG (n=8), while they performed the Milkshake Paradigm. In this paradigm, individuals were cued with an image of either a milkshake or a water solution to be subsequently delivered via a mouthpiece for consumption. It was found that slow frequency power (˜3-8 Hz) in the dlHPC (cluster-based permutation testing using null-distribution cluster size to correct for multiple comparisons) was significantly higher (p=0.002, paired non-parametric permutation testing using channels as observations) in anticipation of a palatable compared to a neutral solution (FIGS. 9B-9D). This profile was observed immediately following the cue (˜110 ms) (FIG. 9D). A later response (˜2-3 sec post cue) was observed in the vmHPC contacts (FIGS. 9B and 9D), in which a significant decrease. however, in slow frequency power (˜5-8 Hz) was observed during anticipation of a palatable solution. These findings suggest that hippocampal slow-frequency power differentially supports anticipation of palatable versus neutral items. Moreover, the temporal dynamics and condition specificity of this hippocampal rhythm point to functionally distinct subregions within the hippocampal area, one of which falls in the hippocampal-LH circuit.

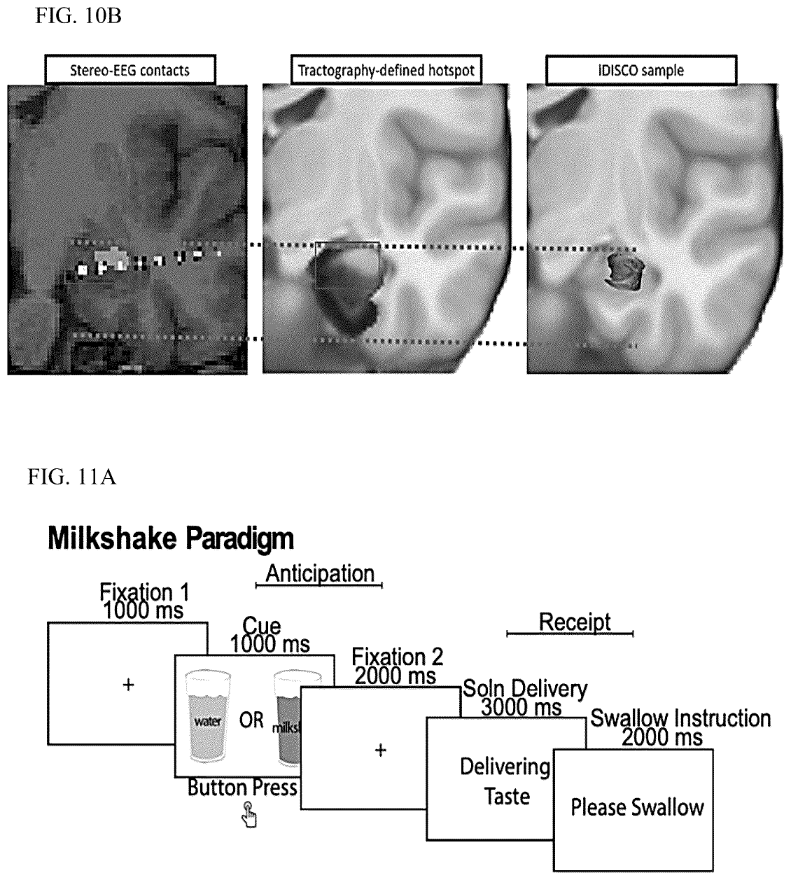

In summary, multimodal circuit-based imaging and neurophysiological analyses (FIG. 10B) implicated the human dlHPC in feeding and binge eating. This subregion can potentially represent a therapeutic target to modulate the feeding circuit for severe obesity and binge eating.

### Example 3: Discriminatory Food Processing in the Insulo-Opercular Cortex

Abstract

The insulo-opercular network is implicated to function critically in gustation. Animal studies have found an involvement of this network to not only encode a sense of taste, but also guide behavior based on anticipated food availability. However, there remains no direct measurement of insulo-opercular activity in humans expecting a taste. Direct intracranial recordings in the insula and frontal operculum in eight human subjects were collected during presentation of visual cues signaling subsequent delivery of palatable or taste-neutral solutions and during ad lib consumption of meals. Cue-specific high-frequency broadband (70-170 Hz; HFB) activity was found predominantly in the left posterior insula and showed preference to taste-neutral cues. In contrast, sparse cue-specific regions in the anterior insula preferred palatable cues. Latency analysis revealed this insular activity was preceded by non-discriminatory activity in the frontal operculum. During ad lib meal consumption, time-locked HFB activity in the left posterior insula at the time of food intake discriminated food types and was significantly associated with task-based cue-specific activity. Thus, it is described for the first time a novel spatiotemporally restricted pattern of neural signals within the human insulo-opercular cortex which underlie the expectant evaluation of food across both controlled and naturalistic settings.

Introduction

To make perceptual inferences and guide behavior, the human brain relies on discriminatory processing of sensory information across domains. Environmental cues which indicate food availability thus drive food-seeking and eating behavior¹. Moreover, the presentation of highly palatable foods such as high-fat and high-sugar items can provoke food intake even in periods of relative energy abundance². Not surprisingly, dysregulation of this process can result in pathologic conditions such as eating disorders and obesity^{3,4}.

A number of rodent studies have shed light on the real-time spatiotemporal dynamics of taste and food processing and its link to eating behavior. Information from various visceral and gustatory inputs has been reported to converge on the insular cortex, which then generates a homeostatic response to food cues⁵. These inputs include the lateral hypothalamus, amygdala and nucleus accumbens⁶⁻⁹. Temporally specific inactivation of the insular cortex using optogenetics abolished cue-provoked food-seeking activity¹⁰. Additionally, human neuroimaging studies have suggested involvement of insular cortex and the overlying frontal operculum in taste evaluation and representation of cues associated with food availability, a process that is dysregulated in the obese state^{11,12,13}.

Although in vivo rodent studies reveal the critical involvement of this cortical region in a process required for survival, they do not readily allow examination of neural processing of complex symbolic and/or language cues encountered in society. Current neuroimaging techniques in humans has limited temporal resolution, thus limiting precise characterization of insulo-opercular dynamics during eating behavior.

The aim of the present study was to address this gap in knowledge by measuring the activity of human frontal opercular and insular cortices underlying food processing. Specifically, there is an interest in a better understanding of the real-time physiologic responses during food anticipation. Both food-specific and topology-specific anticipatory responses within the insulo-opercular cortex were hypothesized. To test this hypothesis, invasive brain recordings were obtained from depth electrodes in epilepsy patients as they performed a measure of anticipatory and consummatory food reward. To assess whether findings related to this task could be generalized to a naturalistic setting, where phases of food anticipation and consummation are not distinctly separated, recordings were also utilized during epochs of ad libitum consumption captured by video recordings of regular meals. It is hypothesized that regions of the insulo-opercular cortex that exhibit food cue-specific activity would also be involved in an expectant evaluation of food during regular meal consumption.

Methods

Participants. Eleven human participants (two female) were implanted with at least one depth electrode in the insular/frontal opercular cortex for electrographic monitoring at Stanford University Hospital. The exact placement of electrodes (AdTech Medical) varied among participants and was determined solely based on clinical grounds following approval of a multi-disciplinary epilepsy surgery review board. Patient characteristics are described in Table 2. All patients provided informed consent as monitored by the Stanford University Institutional Review Board (IRB #: 11354).

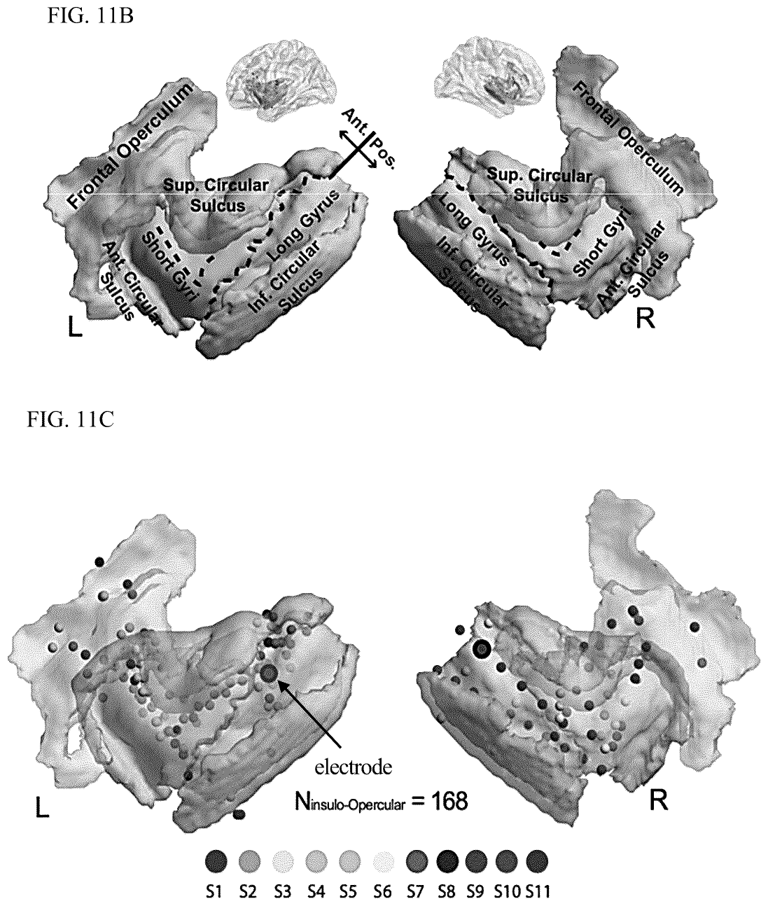

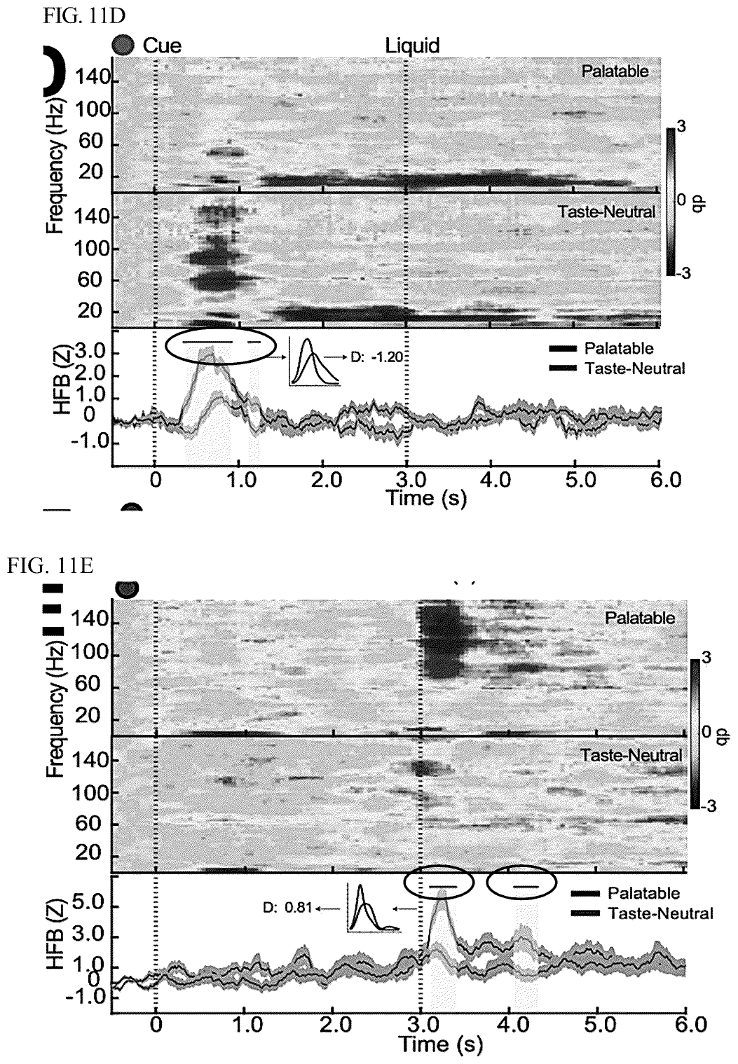

Task Paradigm. Participants completed a food-reward task known as the milkshake paradigm, which is a widely used computer-based task utilized in fMRI research to assay anticipatory and consummatory response to palatable food^{11,14-18}. The task consisted of an anticipation phase and a receipt phase (FIG. 11A). During anticipation, participants saw either an image of a glass of milkshake or a glass of water that served as a visual cue signaling subsequent taste delivery. The cue lasted 1 second (s), followed by an appearance of a fixation cross for 2 s. To maintain participants' attention through the task, they were also instructed to press a button when they saw the cue. At the end of 3 s, the receipt phase occurred, which involved the delivery of either 3 milliliters (ml) of a highly palatable solution (McDonald's Chocolate Shake) or water-based solution. The water-based solution consisted of 25 mM KCl and 2.5 mM NaHCO₃, which was designed to mimic the natural taste of saliva¹⁸. Solution delivery lasted 3 s and was achieved using programmable syringe pumps (BS-8000; Braintree Scientific) attached to 60 mL syringes. Fluids traveled in sterile tygon tubing from syringe tips to a 3D-printed mouth manifold attached to a rigid monitor arm. Participants were subsequently instructed to swallow when presented with “swallow” text. The trial order was randomized during the task, with 80 to 100 trials evenly split between palatable and taste-neutral conditions. Following completion of the task, participants were asked to rate the quality of the palatable solution (Likert scale, 1-10) and which solution (palatable vs taste-neutral) they preferred.

Electrode Registration and Cortical Segmentation. Locations of electrodes in 3D space were extracted from post-implant CT, which were co-registered with patients' pre-operative MRI as described previously¹⁹. Automated cortical parcellation was performed on each individual's MRI to determine anatomical location of the electrodes²⁰. Subregions of the insular and frontal opercular cortices were divided according to the 2010 Destrieux parcellation scheme²⁰: the short insular gyri, the long gyrus and the central sulcus, the anterior segment of the circular sulcus, the superior segment of the circular sulcus, the inferior segment of the circular sulcus, the pars orbitalis, the pars triangularis, and the pars opercularis (FIG. 11B). As the insular cortex has been demonstrated to have a consistent anterior-posterior division²¹, the insular cortex is categorized into anterior-posterior regions relative to the central sulcus. The anterior insular cortex included the short insular gyri, the anterior segment of the circular sulcus, and the superior segment of the circular sulcus, whereas the posterior insular cortex included the central sulcus, long gyri, and the inferior segment of the circular sulcus. The frontal opercular cortex was defined to include the three subdivisions of the inferior frontal gyrus: pars orbitalis, triangularis, and opercularis.

Data Acquisition and Electrophysiological Preprocessing.

Stereoelectroencephalography (SEEG) recordings from implanted depth electrodes were sampled at 1024 Hz. Data preprocessing and analyses were performed using the FieldTrip toolbox²². Line noise (60 Hz and harmonics at 120 Hz and 180 Hz) was attenuated using a notch filter. A laplacian re-referencing scheme of flanking electrode contacts was performed as described previously to minimize far-field volume conducted contributions to the local field potential²³. Time-frequency spectrograms were calculated using Hannings tapers. To extract the high-frequency broadband (HFB) activity, a bandpass filter (Butterworth, two-pass, 8th order) was first applied. Next, the absolute value of the Hilbert transform was obtained to further obtain the analytic signal, which was smoothed using boxcar averaging of 200 millisecond (ms) windows. The analytic signals of delta (1-4 Hz), theta (4-8 Hz), alpha (8-12 Hz), beta(15-25 Hz) and gamma bands (25-50 Hz) were extracted in a similar fashion to the HFB, except 4^{th} order Butterworth filter was used as the bandpass filter. The frequency ranges of these bands were predetermined based on prior literature {24129341}. For task analyses, the data were epoched according to the onset of the cue (−2 to 6 s, cue: 0 s, solution delivery: 3 s) and were Z-transformed against the pre-cue baseline (−0.6 to −0.1 s). For analyses of standard meals, the data were epoched according to the video-stamped time that immediately preceded food entering the mouth (−5 to 5 s, food prior to entering the mouth: 0 s) and were Z-transformed against a pre-timelock baseline (−5 to −4.5 s). Further, for each channel, the effect size between palatable and taste-neutral conditions for the cue period was calculated by averaging band activity between 0 and 1 s, and for the receipt period by averaging band activity between 3 and 4 s.

Cue-Responsive or Receipt-Responsive Site Identification. To determine if a particular electrode was responsive to cues during taste anticipation (0 to 3 s) or delivery (3 to 6 s) during taste receipt, we tested whether the HFB activity in the post-stimulus window was significantly larger than the baseline period (−0.6 to −0.1 s). Differences in the post-stimulus window from baseline were tested in five consecutive non-overlapping 500 ms windows using a cluster-based non-parametric approach²⁴. This was done separately for palatable and taste-neutral conditions. Two-sample t-statistic was obtained at every time point and significant clusters were formed based on temporal adjacency at an alpha level of 0.05. The null distribution for the cluster t-statistic was produced by randomly shuffling data between the baseline window and the post-stimulus window for 1000 iterations and computing the cluster t-statistics. The cluster t-statistic was compared to this null distribution and the post-stimulus period was considered significantly different from baseline using a p-value of 0.05. Subsequently, a channel was considered to be either cue- or receipt-responsive if a significant post-stimulus response of at least 200 ms in any time periods was observed in either palatable or taste-neutral conditions.

Cue-Specific or Receipt-Specific Site Identification. After identification of responsive channels during anticipation or receipt as described above, responsive electrodes were considered either cue-specific or receipt-specific if their activity trace during anticipation (0 to 3 s) or receipt (3 to 6 s) was significantly different between palatable and taste-neutral conditions. To determine differences, a cluster-based, non-parametric approach was employed to compare the HFB activity between palatable and taste-neutral conditions in 500 ms time windows that previously showed a significant post-stimulus response from baseline as defined above. Two-sample t-statistic was obtained at every time point comparing palatable and taste-neutral conditions to form significant clusters based on temporal adjacency at an alpha level of 0.05. The null distribution for the cluster t-statistic was produced by randomly shuffling trials between the palatable and taste-neutral conditions for 1000 iterations and computing the cluster t-statistics. The cluster t-statistic was compared to this null distribution and the time period was considered significantly different between palatable and taste-neutral conditions based on a p-value of 0.05. Lastly, to prevent misclassifying potential large drifts in the signal as different in the absence of any evoked responses, a channel is considered cue- or receipt-specific if the time period determined to be different between the two conditions must overlap with significant post-stimulus time periods by 100 ms. To account for multiple comparisons, the p-threshold was adjusted via Bonferroni correction based on the number of time windows that were used for cluster-based permutation analysis (up to 5). In channels with task-specific response, band power activity during time periods found to be significantly different on cluster-based, non-parametric testing as above, were used to calculate effect size (Cohen's d).

Classification Analysis. To determine if HFB activity was sufficient to differentiate anticipation for palatable or taste-neutral taste on a single trial basis, binary classification was performed using the classification learner toolbox in MATLAB. All classifiers were initially tested using 5-fold cross validation, default classifier parameters and PCA to keep enough components to explain 95% of variance. Of the tested classifiers, K^{th} nearest neighbor (KNN; weighted) yielded the highest performance and was fixed for all subsequent analyses. Briefly, the KNN algorithm involves measuring the distance (euclidean) between the test observation vector and all other prototypes (labeled observations) in feature space. Since the utilized model here is a weighted KNN, weights are applied to the K nearest prototypes, with lower weights applied to more distant prototypes (squared inverse distance weight). Then, the class assigned to the test observation is determined using distance-weighted voting, whereby closer prototypes contribute more to the majority vote. Default model parameters were utilized; euclidean distance metric, squared inverse distance weight and 10 neighbors.

Data from posterior-insula taste-neutral selective electrodes displaying significant increases from baseline were utilized (3 subjects, 6 channels, and 250 taste-neutral and 250 palatable trials). The feature vector was defined as HFB activity in four anticipation time windows: 0-0.5, 0.5-1, 1-2, and 2-3 seconds. These features captured the first and second halves of the cue presentation (0-0.5, 0.5-1 sec) and fixation (1-2, 2-3 sec) periods, thereby approximating the transient and steady state phases of the HFB power response dynamics for each stimulus.

KNN 5-fold cross validation was first performed using the group data (inter-individual classification). Each fold likely contained observations from each individual's class distributions, therefore, the test set was not composed of observations from a single patient whose data was not included in training sets (external validation). A complete external validation was not performed due to a limitation in sample size. After generation of a group KNN model, a separate KNN model was generated for each of the three subjects (intra-individual classification).

Following the cross-validation procedure for the inter-individual classification, permutation testing was performed to assess the significance of the observed classifier performance measures (overall accuracy, and true and false positive rates (TPR, FPR respectively). Statistical testing was performed by generating a new model on data with shuffled values for a single feature between the two conditions. This was done 100 times, and separately for each feature. A p-value was obtained by examining the number of times model performance on the shuffled data was greater than (TPR) or lower than (FPR) the observed value. To investigate differences in model dependency on a particular feature compared to all other features, unpaired permutation testing (1000 permutations) was performed on overall accuracy measures associated with feature in question shuffled data compared to those of permuted data of each other feature, with FDR correction for multiple comparisons.

Response Onset Latency Analysis. The time of onset for the HFB activity was determined using a technique previously described to robustly estimate response onset on a single trial level². Briefly, for a given single trial, contiguous time points of the HFB activity (minimum 100 ms) above 2 standard deviations of the baseline activity were identified. At the first time point above the threshold, a 200 ms window was extracted which was further divided into 10 segments of 100 ms time series with 90% overlap. Linear regression was performed on each of the 20 segments to obtain slope and residual error. Segments with the top five slopes were selected, and the segment with the least mean squared error was defined as the “onset” segment. The first time point of onset segment was used to define the response onset latency (ROL) for the single trial. This procedure was carried out separately for anticipation and receipt periods. For each region of interest, the ROL was computed for all trials, irrespective of palatable or taste-neutral condition, and for all channels that fell within the anatomical region. Hence, the cumulative number of ROL per region is the product of the individual trial and channel numbers.

ad libitum Consumption Analysis. It is hypothesized that regions demonstrating cue-specific response might exhibit similar behavior under a naturalistic setting. Hence, subjects were selected with coverage in regions showing cue-specific response to examine simultaneous video and SEEG recordings as they ate their daily meals. In order to obtain high signal-to-noise (SNR) responses outside of a task paradigm, a consistent behavior during ad libitum consumption is selected for electrophysiologic time-locking. During the process of eating, a universal movement is to bring the food up to the mouth for consumption. Hence, it is defined that the time point immediately preceding food entry (just as the food is about to enter the mouth) as a visual time-lock for off-line analyses. To test this, a meal video segment was chosen for analysis using the following inclusion criteria: (1) the subject's face must be visible in the video to allow for tracking of food movement; (2) the subject must be eating a meal which consists of at least two types of food (e.g. entrée or dessert) for comparison, such that each one may serve as a control for common motor signals that may be time-locked; (3) there must be at least 10 repetitions in each food type to allow for trial-averaging; (4) there must be no seizures or epileptic activity both 5 hours before and after the video file to avoid preictal or postictal activity; (5) when multiple videos are eligible, the video meal segment that was closest in time to when the task recording was performed was chosen to avoid mismatch in signal quality between task and naturalistic conditions²⁶. Two-independent reviewers (AF, RS) evaluated the eating content of the video segment by time-stamping the immediate timepoint prior to every food bite.

Identification of Food Specific Sites during Natural Eating. To determine if there was a significant food-specific response in the insular/frontal opercular cortices during regular meals, the time-locked HFB activity was compared between two food types near the time preceding food entry into the mouth. Due to quality of the recorded videos, only entrée versus non-entrée food items were clearly discernable. Non-entrée food items included either pudding or fruit. To compare the HFB activity between two food types, cluster-based non-parametric testing was used to obtain two-sample t-statistic at every time point from −1 s to 1 s between entrée or non-entrée trials. Significant clusters were formed based on temporal adjacency at an alpha level of 0.05. The null distribution for the cluster t-statistic was produced by randomly shuffling trials between the entrée and non-entrée trials for 1000 iterations and computing the cluster t-statistics. The cluster t-statistic was compared to this null distribution and the time period was considered significantly different between the two conditions based on a p-value of 0.05. Food type-specific HFB activity response during meal consumption was investigated in effort to control for potential stereotypic responses representative of motor system activation leading up to food bites. Finally, to understand what the difference in the HFB activity between eating two food types may represent, a chi-squared analysis was performed to test for associations in the responses between task-based and ad libitum consumption.

Results

Eleven subjects participated in the task paradigm (Table 2). All subjects were right-handed. All subjects reported they preferred the palatable solution over the taste-neutral solution, with an average rating of 6.1.

Dynamics of Neural Activity During Task Paradigm

The task consisted of two phases: an anticipatory phase that evaluates neural representation of food expectation and a receipt phase that assesses neural activity of the sensory and evaluative aspects of food consumption (FIG. 11A). SEEG electrode recording was performed during these two phases with coverage of bilateral insular and frontal opercular cortices across eleven subjects (FIG. 11C). There were 103 electrodes in the left hemisphere and 65 electrodes in the right hemisphere with a total of 168 electrodes covering the insulo-opercular cortex. In the insular cortex, electrodes encoding the robust HFB responses to cue or receipt of the solution were found (FIGS. 11D and 11E). For example, in the left posterior insula, an increase was observed in the HFB activity within is of cue onset, which was greater in response to the taste-neutral cue compared to the palatable cue (FIG. 11D, p<0.05, cluster-based permutation testing, alpha <0.05, d=−1.20). This was also evident on time-frequency spectrogram. Interestingly, although there was a strong response to the cue at this site, there was no activity upon solution delivery. At a separate site in the anterior insula, instead of an anticipatory response, an increase was observed in the HFB activity at 3 to 3.5 s, corresponding to the time of solution delivery. The palatable solution elicited greater increase in the HFB activity upon receipt (FIG. 11E, p<0.05, cluster-based permutation testing, alpha <0.05, d=0.81).

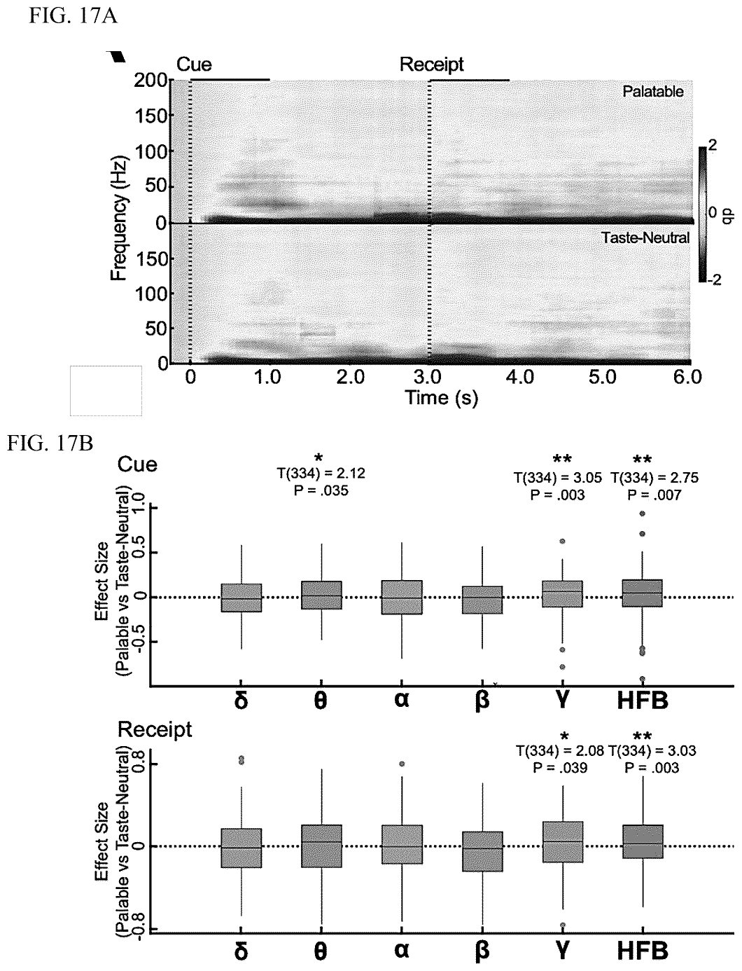

To examine the overall activity of the insulo-opercular cortex during the task, the time-frequency spectrogram was also computed by averaging across all electrodes (N=168, FIG. 17A). Again, an increase was observed in broadband power primarily within the first second following either cue presentation (onset at 0 s) or solution receipt (onset at 3 s). There was qualitatively higher broadband power roughly 25-200 Hz in the palatable condition as compared to the taste-neutral condition after cue and receipt. To quantify the difference in neural activity between these two conditions, the effect size in the six traditional band power frequencies and across all channels in the first second of stimulus onset was calculated (FIG. 17B). In the anticipatory period, the palatable, compared to taste-neutral, cue elicited significantly greater average insulo-opercular activity in gamma (p=0.003, two-sample t-test, t (334)=3.05) and high-frequency broadband powers (p=0.007, t (334)=2.75). Out of the lower frequency power ranges, only the theta range was associated with higher activity in the palatable condition (p=0.035, t(334)=2.12). Similarly in the receipt period, delivery of palatable, compared to taste-neutral, liquid to the subjects' mouth resulted in higher insulo-opercular activity in gamma (p=0.039, two-sample t-test, t (334)=2.08) and high-frequency broadband powers (p=0.003, t (334)=3.03). Overall, these results suggest discriminatory neural activity exist in the insulo-opercular region and can be captured by the high-frequency power ranges. Given HFB activity has been shown to directly correlate with underlying spike activity, HFB activity is used in the remaining analyses.

Topology of Insular and Frontal Opercular Responses

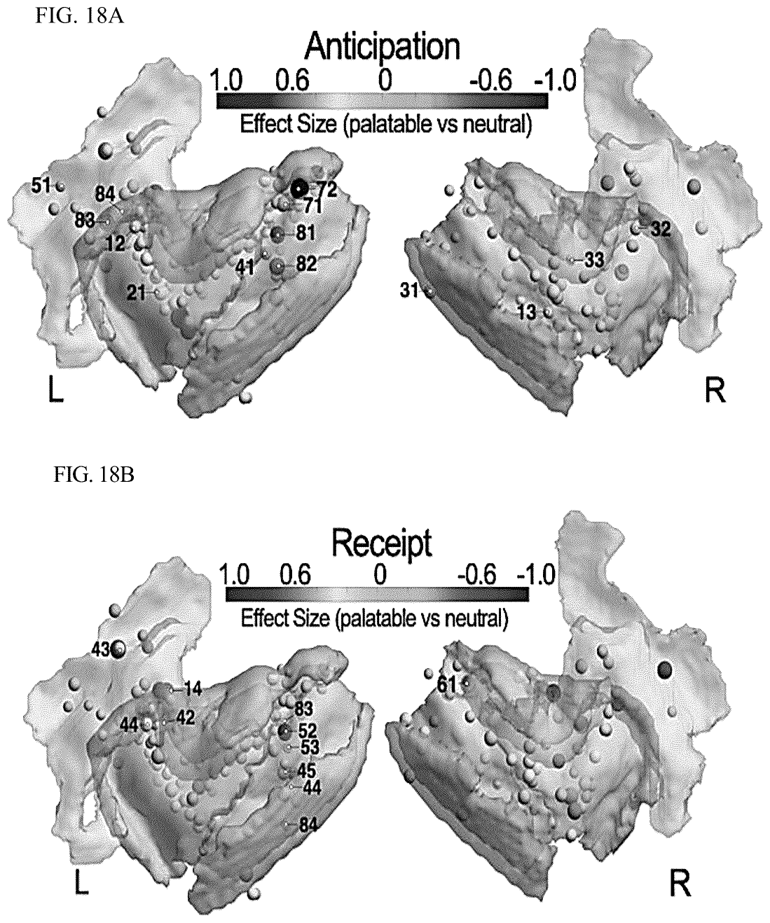

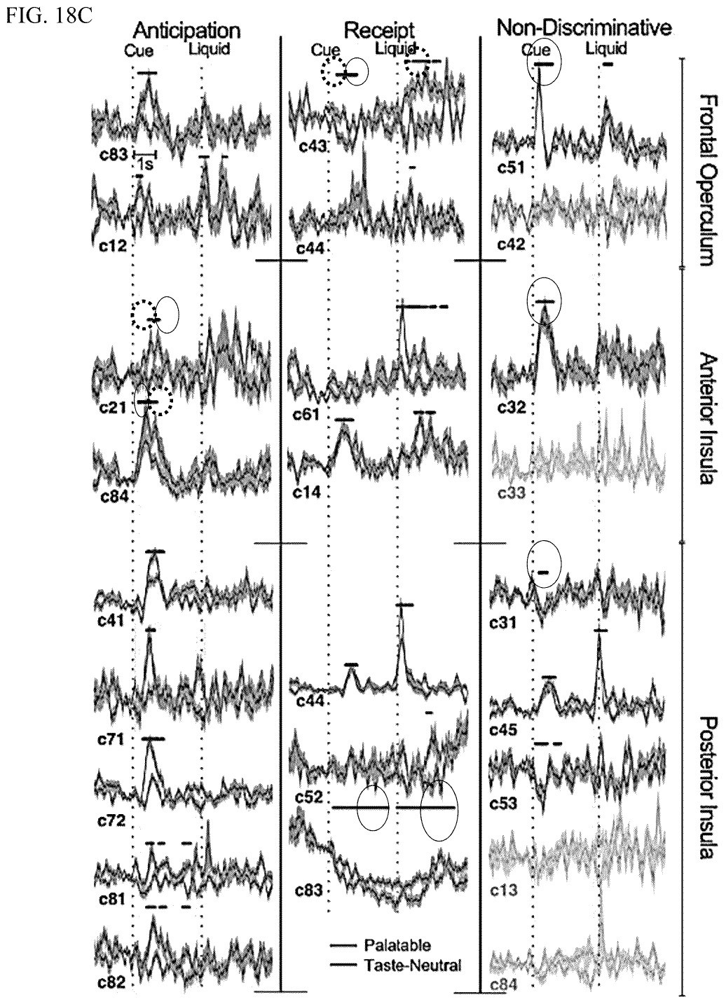

Given the overall increase in insulo-opercular activity in the palatable condition (FIGS. 17A and 17B) but variable pattern of response profile in individuals channels (FIGS. 11D and 11E), site-by-site responses driving the group discriminatory activity in the insulo-opercular cortex are to be determined. The HFB effect size map on a per channel basis was qualitatively visualized and automatic cortical parcellation was used to determine cortical subregions (FIGS. 18A-18C). In the anticipatory period, higher HFB was observed for the palatable cue (mostly warm colors) as compared to the taste-neutral cue (FIG. 18A). Interestingly, there was a localized area of cold colors in the left posterior insular along the dorsal central sulcus, denoting higher HFB activity for the taste-neutral cue. In the receipt period, a heterogenous distribution of discriminatory HFB profile was observed, with a predominance of channels showing higher HFB activity for the palatable cue (FIG. 18B). Next, the corresponding HFB activity trace of the labeled electrodes (FIG. 18C) was visualized to show examples denoting the variation in pattern of discriminatory responses in the insulo-opercular cortex. These traces were stratified in three columns by whether the electrode was cue-specific, receipt-specific or non-discriminatory/inactive. Again, during the anticipation period, HFB activity generally increased to peak within the first second (FIG. 18C, first column). Notably, the left posterior insular responses were largely uniform during anticipation, showing lower HFB activity to the taste-neutral cue as compared to the palatable cue. In contrast, the HFB activity trace in receipt-specific were largely heterogeneous, characterized by variable peak latency and waveform morphology (FIG. 18C, second column). For reference, the HFB activity trace of cue-responsive but not cue-specific sites and of non-active sites was visualized (FIG. 18C; third column).

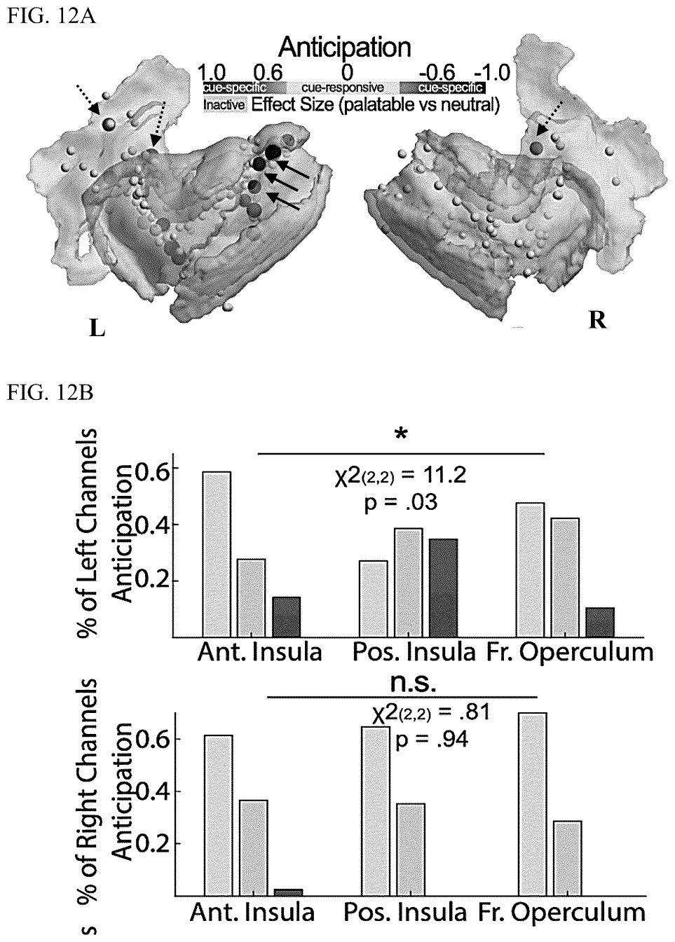

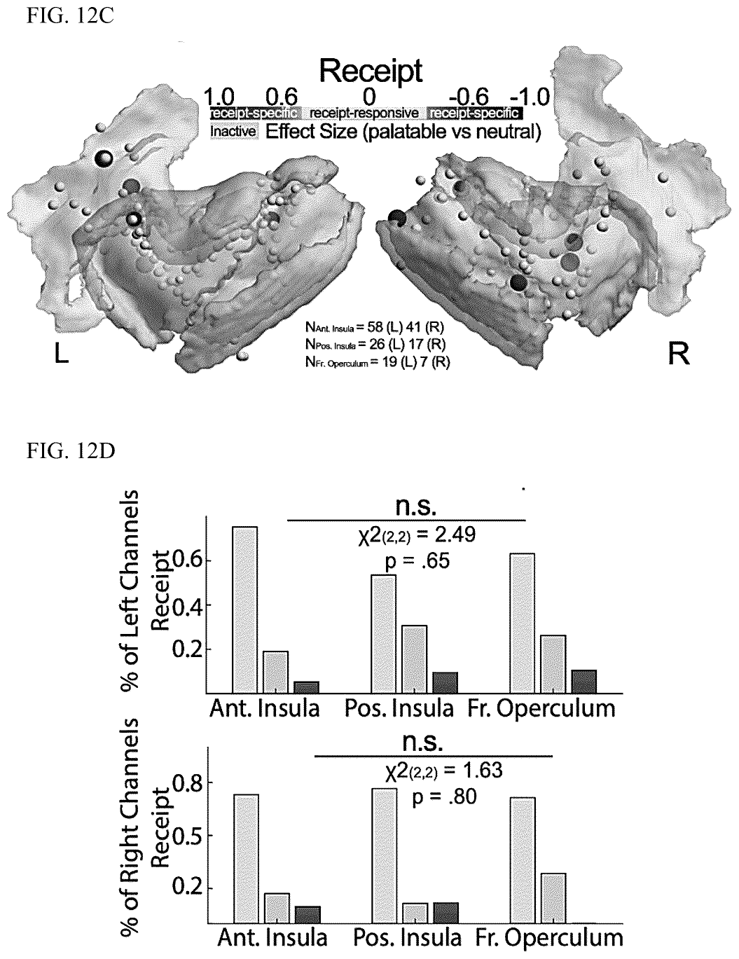

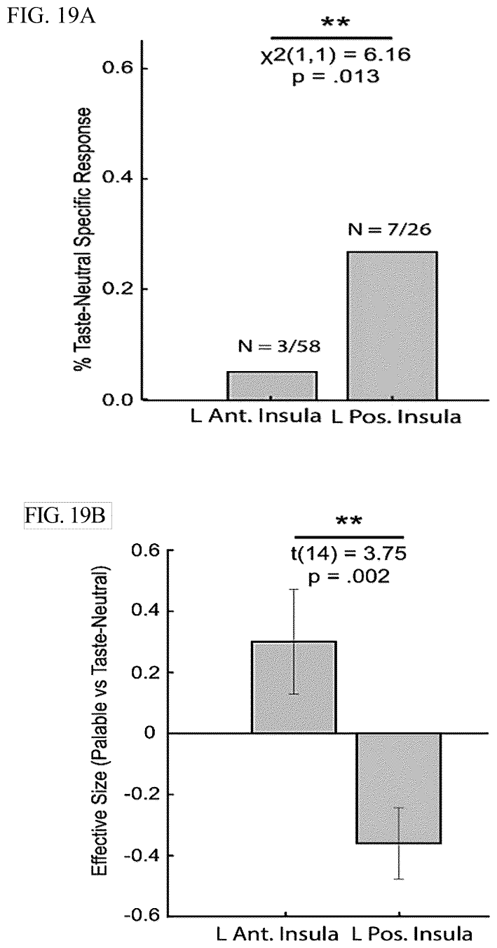

To understand the topology of insulo-opercular activity, a cluster-permutation-based threshold of HFB activity was applied to categorize each electrode as cue/receipt-responsive, cue/receipt-specific or non-active (FIGS. 12A-D). During task anticipation, a cluster of cue-specific sites showing greater activity for taste-neutral conditions (cold colors, such as blue spots indicated by the solid arrows) along the dorsal aspect of the left posterior insula was found (FIG. 12A left panel). In contrast, the left anterior insula showed several channels with cue-specific activity biased towards palatable conditions (warm colors, such as red spots indicated by the dotted arrows). The right insular and frontal opercular cortices showed minimal cue-specific activity, with only one channel noted to be cue-specific (FIG. 12A right panel, indicated by the dotted arrow). The left insulo-opercular region was observed to have a significantly different distribution of cue-responsive, cue-specific or non-active channels, with the highest percentage of cue-specific channels found in the left posterior insula (FIG. 12B top panel; shades of blue (the top section of the right-most bar in each region) or red (the bottom section of the right-most bar in each region) electrodes; p=0.03, chi-squared test, χ2 (2,2)=11.2). Specifically, the left posterior insula cortex was associated with the highest proportion of taste-neural cue-specific sites (FIG. 19A; p=0.013, χ2 (1,1)=6.16). Amongst cue-specific channels in the anterior and posterior insula, the effect size between palatable and taste-neutral conditions was significantly different within these two insular subregions (FIG. 19B; p=0.002, two-sample t-test, t(14)=3.75). The left posterior insula showed greater activity to the taste-neutral cue, whereas the left anterior insula showed greater activity to the palatable cue. On the right insulo-opercular side, there was no difference in the proportion of distribution in cue-responsive, cue-specific or non-active sites (FIG. 12B bottom panel; all p>0.05, shades of blue or red electrodes; chi-squared test).

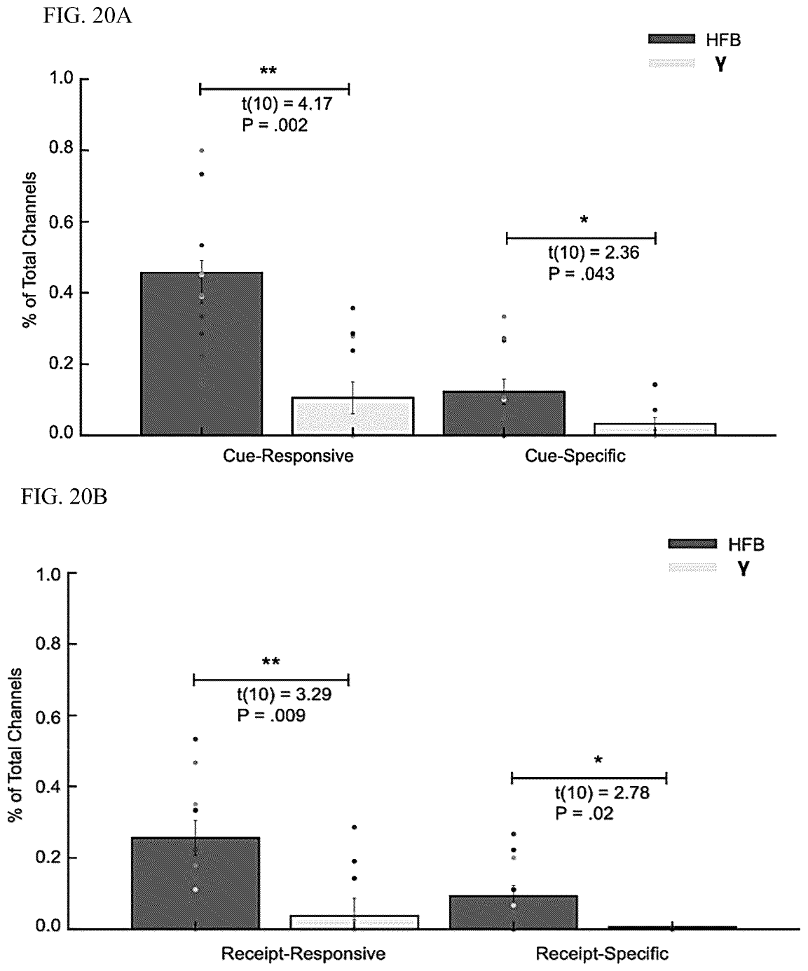

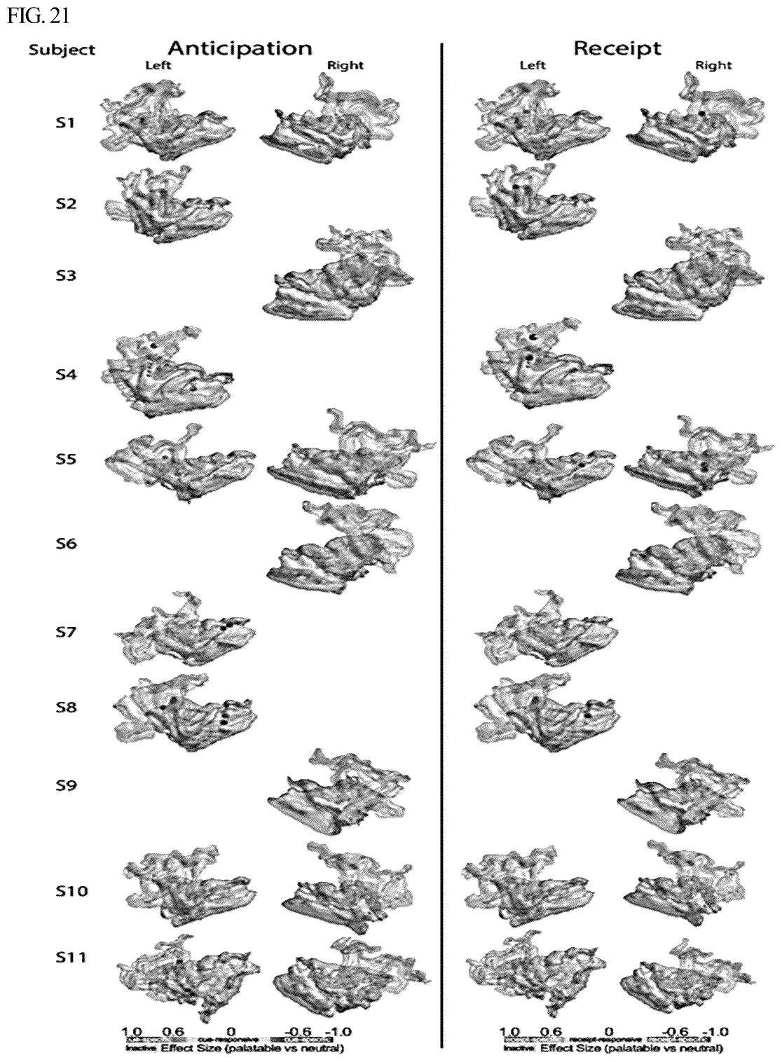

Next, the distribution of responses during task receipt was examined (FIG. 12C). Receipt-responsive and receipt-specific HFB activity was found in a distributed pattern across bilateral insular and frontal opercular cortices. Distribution of receipt-responsive, receipt-specific and non-active channels were distributed in an equal manner across the anterior insula, the posterior insula and the frontal operculum (FIG. 12D; p>0.05, chi-squared test). Additionally, Applicant computed the proportion of task-responsive and task-specific channels using gamma power (25-50 Hz) as average insulo-opercular activity also indicated higher gamma power in the palatable condition. Compared to HFB activity, significantly lower number of channels were found to be cue-responsive (FIG. 20A; p=0.002, paired t-test, t(10)=4.17) or cue-specific (p=0.043, t(10)=2.36). Similarly, the number of receipt-responsive (FIG. 20B; p=0.009, t(10)=3.29) or receipt-specific (p=0.02; t(10)=2.78) channels were also significantly lower using gamma power.

Individual subject topology is available in FIG. 21. Additionally, MNI coordinate of every cue-specific and/or receipt-specific electrodes has been provided in Table 3.

Finally, neural responses to food have been previously reported to depend on individual body mass index (BMI)¹¹. It is thus investigated if the proportion of responses found in the current study cohort was confounded by individual BMI and rating of milkshake. Neither the proportion of cue-specific or cue-responsive channels were associated with subject BMI or post-task rating of milkshake (Pearson's Correlation test; all P>0.05; Table 3). Additionally, no relationships in receipt responses and subject attributes were found (Pearson's Correlation test; all P>0.05; Table 3). Similarly, no correlation between age of subjects and task responses were found (Pearson's Correlation test; all P>0.05; Table 3).

Taken together, evidence of food-specific cue encoding predominantly in the left posterior cortex was identified. The left posterior insula cortex tended to favor taste-neutral cue whereas the left anterior insular cortex showed preferential activity for the palatable cue. The encoding of solution receipt was more heterogeneous across the insular and opercular cortices.

Classification of Anticipatory Response on a Single Trial Basis

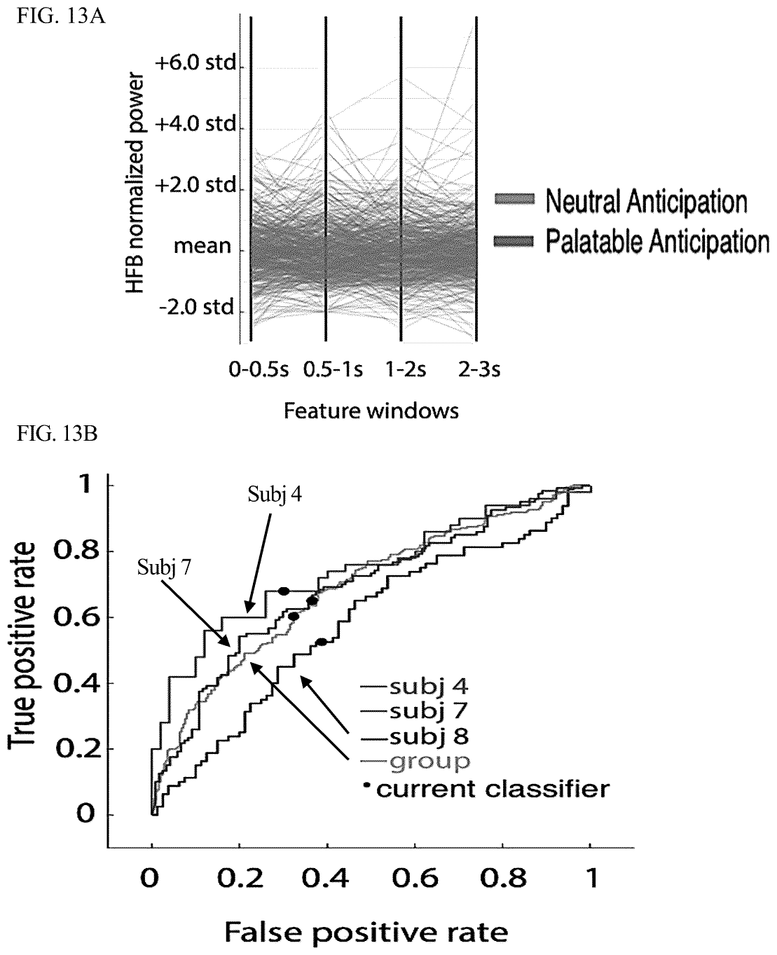

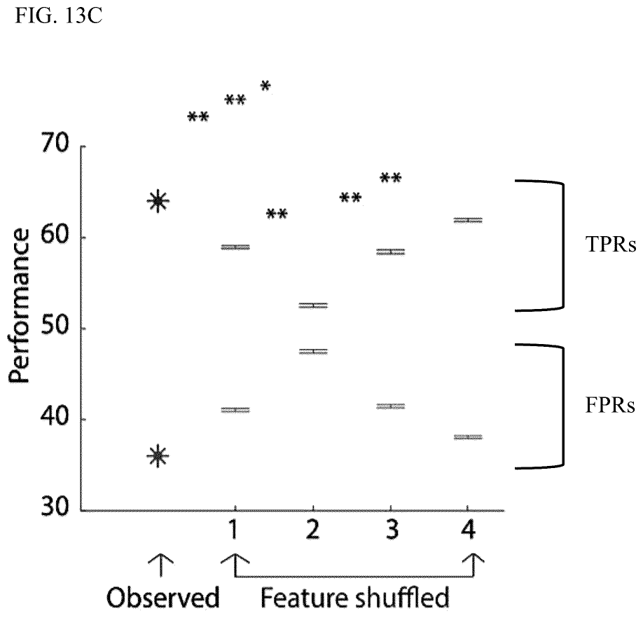

Taste-neutral HFB responses were relatively localized to the posterior insula. It was therefore tested whether posterior insular HFB activity was sufficient to classify between the two anticipatory conditions on a single trial basis (FIGS. 13A-13C). To do so, a weighted KNN classifier was trained and tested on data from responsive (displaying significantly increased HFB power from baseline) and selective (displaying significantly higher power for taste-neutral compared to palatable anticipation) posterior insula channels. The parallel coordinate plot displays all utilized observations (n=500) stratified by palatable and taste-neutral trials, as a function of the features utilized (FIG. 13A). A 4-D feature vector used which included mean HFB power averaged in four anticipatory time epochs (FIG. 13A; 0-0.5 s, 0.5-1.0 s, 1.0-2.0 s, 2.0-3.0 s). PCA was used for dimension reduction; after training, four components were kept to explain 95% of the variance (explained variance per component, in order, was 61.8%, 16.3%, 13.6%, and 8.2%). Notably, a cluster of taste-neutral observations in the 0.5-1.0 s feature window was observed (FIG. 13A).

Inter-individual (group) classification performance yielded 64% mean TPR and 37% mean FPR across the two classes (FIG. 13B), with an AUC of 69%. An individual subject model was also generated, separately for each of the three subjects. Mean TPR and FPR and AUC for each of the three subjects were (4) 69%, 31%, and 74% (7) 64%, 36%, and 69% and (8) 57%, 43%, and 58%, respectively (FIG. 13B). Note that for all classifiers, mean TPR across classes and overall accuracy are equal due to a matched observation number in both classes. The classifier depended significantly on three of the four time periods (p<0.01, p<0.01 and p=0.01, respectively), while reliance on the fourth time period did not reach significance (p=0.08). Classification performance was significantly more diminished when permuting the second time period compared to any other time period (FIG. 13C; p=0.002 feature 2 vs. 1, p=0.002 feature 2. vs 3, p=0.002 feature 2. vs 4, P_{FDR}=0.002). This suggests that HFB activity during food anticipation is sufficient to classify between palatable and taste-neutral conditions, with the 0.5 to 1.0 s period following a food cue being most contributory to classification.

Sequence of Insular and Frontal Opercular Activity

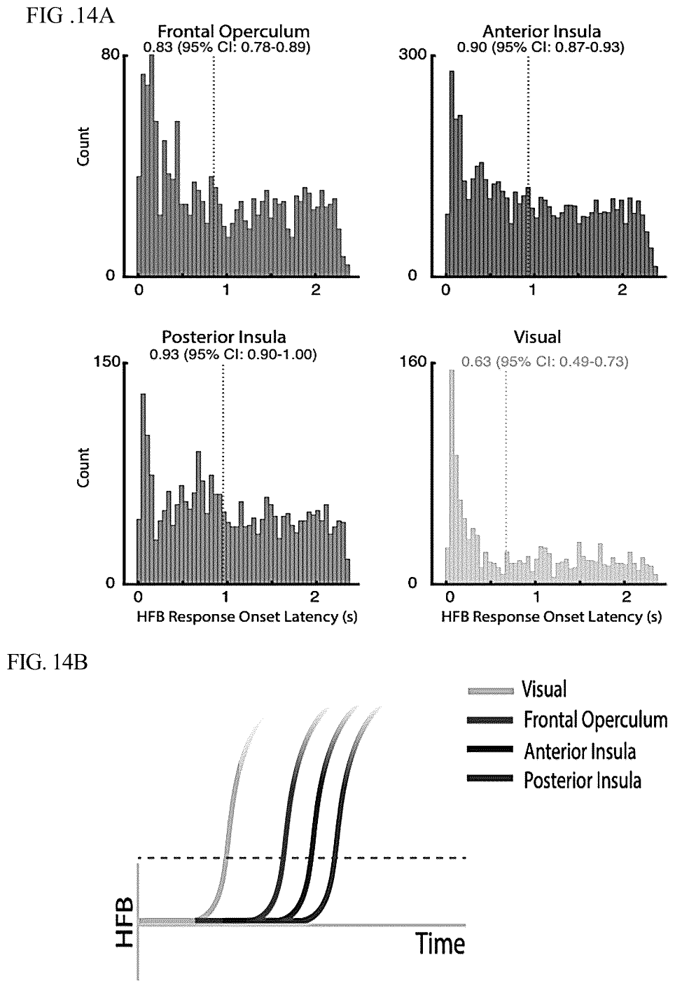

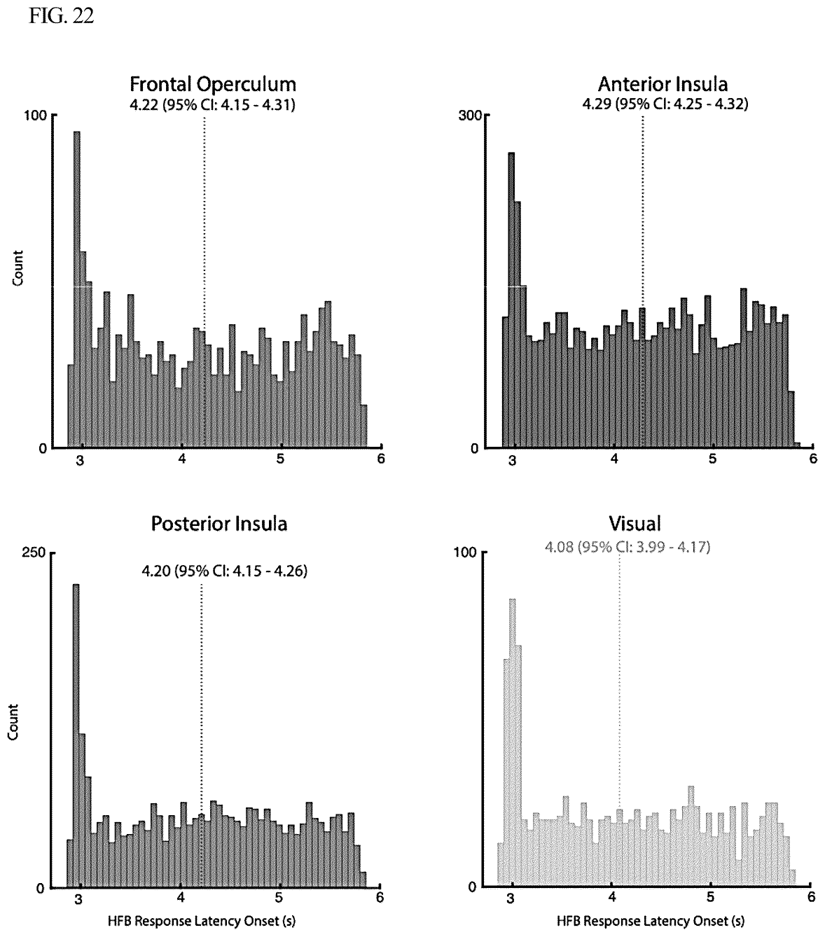

Next, to better define the latency of HFB activity at each anatomic region, the HFB response onset latency (ROL) was characterized during anticipation and receipt. Specifically, using a single trial approach, the HFB ROL at the frontal operculum, the anterior insular cortex, and the posterior insular cortex were determined. During anticipation, earlier response onset in the frontal operculum (FIG. 14A, median, 95% CI: 830 ms, 780-890 ms), compared to the posterior insular cortex (920 ms, 890-960 ms), was found. The anterior insula had a response onset in between (920 ms, 890-960 ms). As a control, the HFB ROL in visual sites was determined, which would be expected to respond to the visual cue²⁵. As expected, the visual sites showed the earliest onset time (640 ms, 500-730 ms). In contrast, during task receipt, the distribution of the HFB ROL across sites was flat with no obvious peak onset time (Frontal Operculum: 4220 ms, 4160-4300 ms; Anterior Insula: 4290 ms, 4250-4320 ms; Posterior Insula: 4200 ms, 4150-4260 ms; Visual 4080 ms, 3990-4170 ms; FIG. 22).

Naturalistic Examination of Dynamic, Time-Locked Neural Activity During Eating

As most human studies involving feeding behavior are performed within the confines of a controlled task setting, little is known about how activity during a task may be relevant to neural dynamics during eating in the natural setting. As subjects in the epilepsy monitoring unit were continuously video monitored, this afforded a unique opportunity to investigate neural activity when subjects consumed meals during their hospital stay. It is hypothesized that the left posterior insular cortex, which was found to be consistently cue-specific during task anticipation, may encode similar activity during consumption of a regular meal.

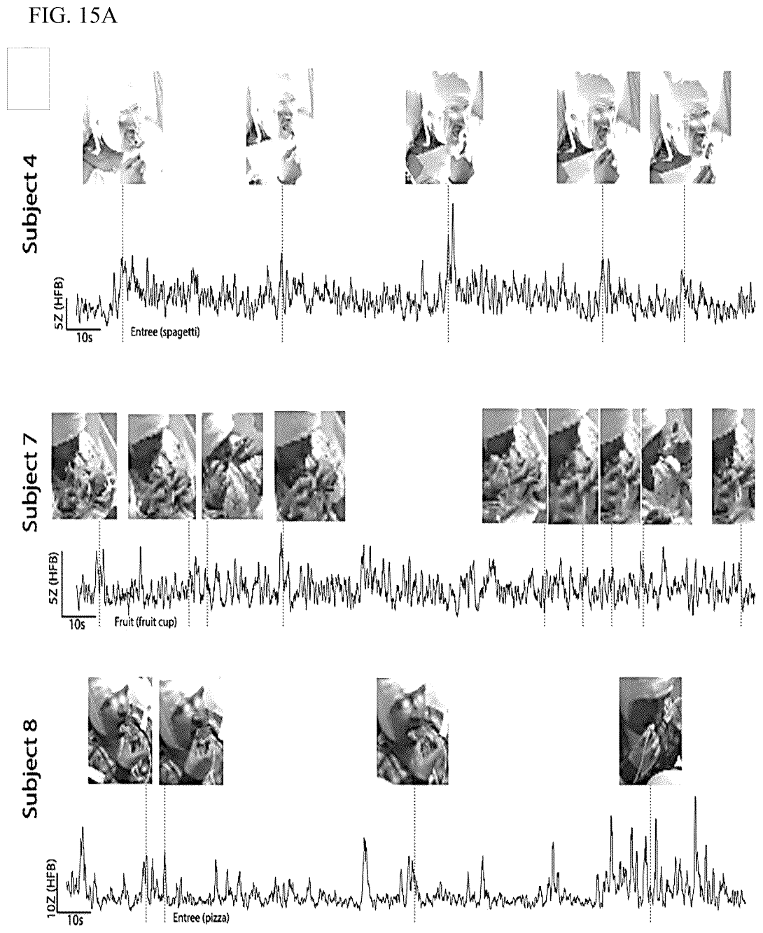

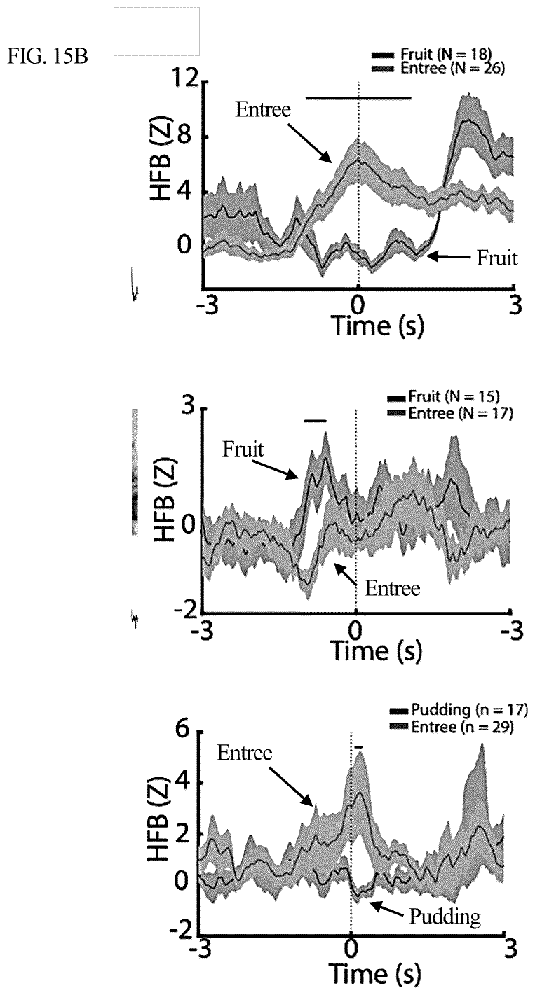

Three subjects (subjects 4, 7, 8) were identified with at least two or more cue-specific channels in the left posterior insular cortex. In these three subjects, video segments for analysis of ad libitum consumption were identified (FIGS. 15A and 15B). It is reasoned a repeated anticipatory moment during eating might be when food is brought from the plate to the mouth, at which time subjects might preemptively perceive the various sensory aspects of the food. As such, the time point immediately preceding food entering the mouth was used as a time-lock (FIG. 15A; vertical dotted lines). In the included meal sessions, subject 4 took 26 bites of an entrée dish (rice with assorted vegetables and meat) and 18 bites of a fruit cup; subject 7 took 17 bites of an entree dish (pasta with assorted meat) and 15 bites of a fruit cup; subject 8 took 29 bites of an entree dish (pizza) and 17 bites of a pudding. In the continuous HFB activity trace, an HFB increase at the time of food entering the mouth was observed (FIG. 15A). However, an increase in the HFB activity near the time of food entering the mouth can be attributed to a number of factors, such as motor behavior of raising one's arm, or the act of opening one's mouth. These behaviors are likely to occur at the chosen time-lock. Thus, to evaluate food-specific responses in the insular cortex, the time-locked HFB activity between eating two types of food in the same meal was compared (FIG. 15B). Interestingly, significant differences in the time-locked HFB activity after individual bites of an entrée dish as compared to eating a non-entrée dish across the three subjects (p<0.05, cluster-based permutation testing, alpha <0.05) were found. These sites were thus characterized as “food-specific”.

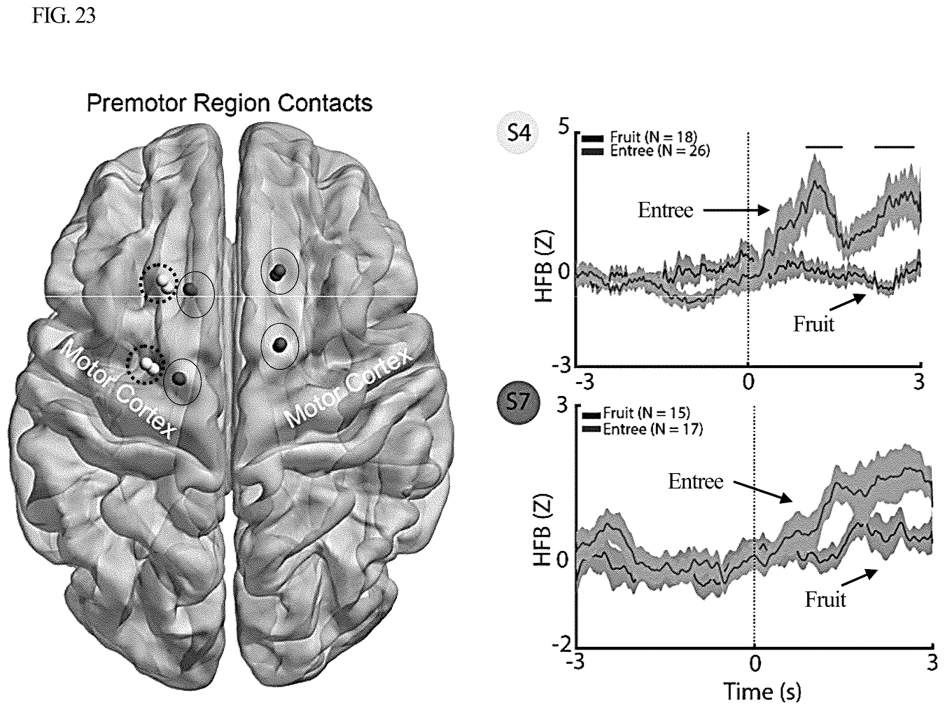

To assess if the changes in HFB near the time of food intake can be attributed to motor differences such as slight differences in arm positioning for eating various food, electrodes positioned in the pre-motor region were identified (FIG. 23). No electrodes were found in actual motor cortex in this limited cohort of subjects as depth electrode traversing the motor cortex is generally avoided. In both subject 4 and 7, there were no pre-bite differences in HFB activity in the pre-motor region between food groups. Not surprisingly however, there was a dissociation in HFB activity after chewing, which likely reflects food-specific motor differences during chewing.

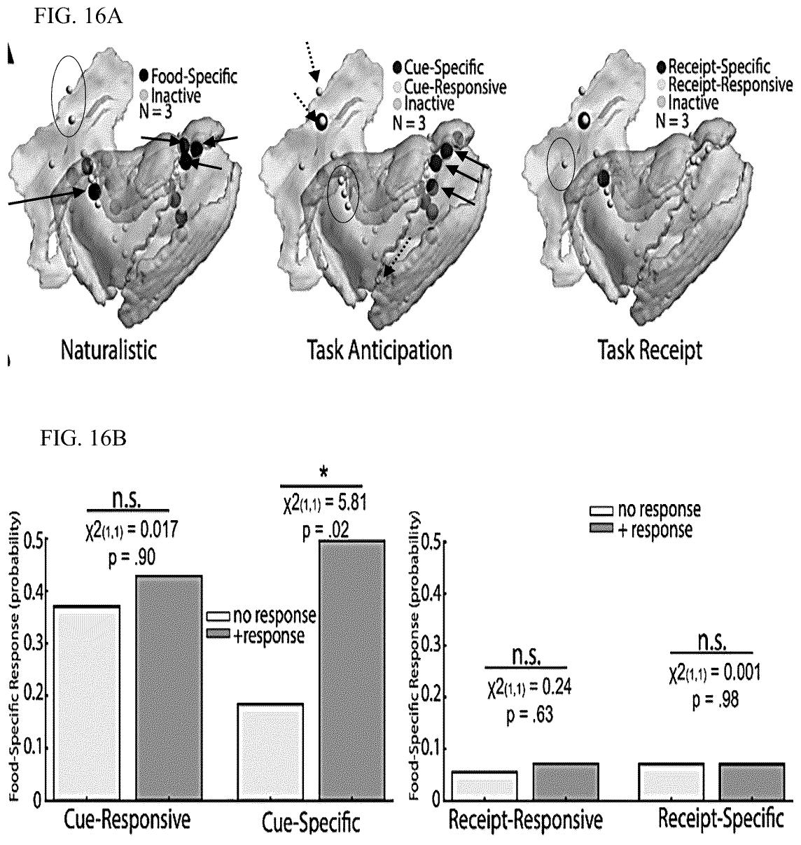

To understand what the food-specific sites might represent, task responsive sites were compared with food-specific sites during a regular meal (FIGS. 16A and 16B). The sites of food-specific eating responses and cue-specific responses were similar (FIG. 16A, indicated by solid arrows in the left and the middle panels, respectively). Indeed, food-specific sites were significantly associated with regions demonstrating cue-specific responses (FIG. 16B, p=0.02, chi-squared test, χ2 (1,1)=5.81). Sites that were only cue-responsive were not associated with a food-specific eating response (FIG. 16B; p>0.05, chi-squared test). Additionally, receipt-responsive or receipt-specific sites were not associated with food-specific eating responses (FIG. 16B; p>0.05, chi-squared test).

Discussion

The principal objective of this study was to better understand the involvement of the insulo-opercular cortical representations in eating behavior. SEEG electrode recordings were used during a task integrating food delivery designed to elicit anticipatory and consummatory responses to food. Evidence was found for food-specific encoding in the left posterior insular cortex during anticipation but distributed and heterogeneous responses in the bilateral frontal opercular and insular cortices during receipt. Specifically, the left posterior insula showed increased activity for taste-neutral cue. In contrast, sparse cue-specific channels in the left anterior insula showed selectivity towards the palatable cue. Single trial classification using posterior insula HFB power dynamics during the anticipatory period yielded 64% mean TPR (and accuracy) and 69% AUC in classifying the item of anticipation. Latency analysis revealed early activation in the frontal operculum which was minimally discriminatory to food type, but this was followed by insular activation with high food-type specificity. During ad libitum meal consumption, time-locked differences in the HFB activity at the time of food was about to enter the mouth was observed between food types. In the same subjects, this response was associated with task response during anticipation, but not during receipt, providing support that the posterior insular cortex is intimately involved in food-specific expectations under both task and natural contexts. To the best of knowledge, this study represents the first-time the human insulo-opercular cortex has been directly measured during food intake.

Although the insulo-opercular cortex is generally considered the site of the primary gustatory cortex^{12,27}, mounting evidence suggests the insular cortex is involved in processing of food expectation^{10,28-30}. Several studies of single-unit activity in the insular cortex of rodents have shown neurons in the area can be excited or inhibited during an auditory cue that signals food availability^{10,31,32}. Additionally, modulation of insular activity during a cue can subsequently alter food-oriented behavior¹⁰. Results provide evidence that the human insular cortex shares similar features of cue processing. Specifically, it was observed that the palatable cue could elicit both higher and lower HFB activity compared to the taste-neutral cue. Interestingly, the majority of the cue-specific responses were localized to the left posterior insula with lower HFB activity during the palatable cue. Given cue-specific HFB activity was able to correctly predict subsequent trials, there may be a translational opportunity leveraging food-specific electrographic activity to guide a neuro-modulatory approach for pathologic eating behavior, as prior preclinical studies have attempted^{10,33}.

A secondary objective of the study was understanding the responses following food receipt. It is important to note that the consummatory response observed in the task paradigm is a composite sensory response, encompassing factors including taste, viscosity and temperature. The results are consistent with recent neuroimaging and electrophysiological studies that have found taste-responsive regions in the insular and opercular corticesi^{12,34,35}. In particular, 7T fMRI studies^{12,35} of taste regions in the insular cortex suggested taste encoding might be a distributed process with no insular topography specific to tastes. This is consistent with the finding that the discriminatory response during the receipt period is largely homogeneously distributed across the insulo-opercular cortex. On the contrary, the study suggests that cue-representation in the human insula is likely more localized. Evidence was found that the posterior insula showed greater HFB activity for taste-neutral conditions, whereas the anterior insula showed greater HFB for palatable conditions. Interestingly, the posterior insula has been reported to respond more generally to aversive stimuli³⁶ and hence the taste-neutral solution in the study may represent a relatively more aversive stimulus than the palatable solution. Similar to these studies, it was found that the receipt responses were less localized and distributed across the insular and opercular regions. As milkshake has mixed macronutrients compared to the taste-neutral solution, one cannot rule out that the wide distribution of responses was due to regional encoding of other sensory aspects of the liquid presented such as viscosity and temperature.

It needs to confirm if the observed task responses are relevant to a more naturalistic setting. As neuroimaging experiments rely on a task design for repeated scans for high SNR and are limited by the confined scanner space, there is currently no knowledge of the activity of the human insular cortex during ad libitum eating. Taking advantage of the high temporal resolution of SEEG recordings, high SNR was found by using the time point immediately preceding food entering the mouth as a natural time-lock. Indeed, differential neural activity were found when the subject was eating an entrée as compared to non-entrée items including fruit or pudding. Additionally, regions that showed discriminatory activity were significantly associated with anticipatory activity during task, but not receipt activity. These results suggest that the insular cortex is predictably involved in preemptive evaluation of the food a person consumes prior to each bite.

The laterality of insular function in food processing is debated³⁷. Evidence was found to support insular laterality, as the left posterior insula has previously shown anticipatory cue-specific HFB activity changes. Similarly, several task-based fMRI studies have demonstrated asymmetric responses. One study found the left posterior insula's response to food images was associated with serum glucose levels³⁸. Another has revealed preferential left frontal operculum activation with symmetric (tip-of-tongue) application of taste stimuli³⁹. In right-handed subjects, the left (dominant hemisphere) inferior insular region has been associated with taste-stimuli associated activation⁴⁰. However, primate studies have suggested ipsilateral stimulation of nerves involved in taste sensory perception lead mainly to ipsilateral insular-opercular activation⁴¹. In humans, the precise connectivity of taste receptors with the insular-opercular cortices is unknown, complicated by dense innervation of the tongue gustatory papilla and other oral sensory surfaces by branches of cranial nerves V, VII, IX, and X⁴². The functional necessity of insular processing has been suggested by individual reports of patients with left posterior insular stroke⁴³, leading to differential laterality in sensitivity to variations in taste intensity. These studies suggest aspects of food-related processing may be lateralized in the human insular cortex.

Prior work using gustatory and/or cue-based tasks have generated insular parcellations based on functional results. Huerta el al.⁴⁴ aggregated the results of three food-cue paradigms, which includes the milkshake task. This study identified the left anterior insula as more consistently responsive to food cues. Similarly, a recent fMRI study incorporating food cues in addition to concurrent glucose sampling found the left dorsal insula as cue-responsive, and specifically sensitive to circulating glucose level³⁸. Finally, from a structural connectivity perspective, Ghaziri et al.⁴⁵ found that the left dorsal anterior and posterior insula is connected to the nucleus accumbens, a reward center heavily implicated in modulating hedonic aspect of food intake. The present study adds to the literature by again demonstrating predominance of the left insula in responding selectively to food cues. Specifically, the left mid to posterior insula was found as primarily selective, which overlaps with the ROI in many of these studies.

There is growing evidence that activity in the insular cortex is gated by physiologic needs⁵. It was not able to control for the effects of hunger and satiety on insular responses. To minimize these confounds, the task was conducted generally in the afternoon (13:00-17:00) prior to dinner. Further, while hunger ratings were not measured, food orders were initiated and placed by the patient during standard meal times. Future naturalistic studies with built in questionnaires to assess subjective satiety level and meal rating, as well as electromyography and eye tracking should be pursued to provide additional levels of behavioral control. In addition, the study cohort consists of individuals with medication-intractable epilepsy, and as such, cognitive processes may not generalize to the healthy population. Nevertheless, the regions of interest in the study were confirmed to be outside the seizure onset zone. In addition, similar responses were observed in individuals with different epilepsy types and severities, which support the generalizability of the present findings. Only one patient in the sample was female, which may limit generalizability due to potential sex differences. However, prior neuroimaging studies have been conducted on female samples with similar findings⁵, suggesting that these results in male patients converge with data from female samples. Lastly, electrodes were placed according to clinical indication for seizure mapping directed by safely accessible trajectories to sample insulo-opercular cortices. Thus, anatomic variations in the regions sampled exist and are unavoidable in a study of this type. According to clinical need, not all patients had bilateral or symmetric coverage of the anterior and posterior insular or associated opercular structures. To mitigate this, group and individual subject analyses were performed, which revealed consistent activity in the left posterior insula on both a group and individual level.

Here, to the best of the knowledge, insular and opercular electrophysiology underlying food intake was characterized for the first-time. The results of a broadly used task paradigm were extended to aspects of ad libitum eating, suggesting an integral role of the insular cortex in expectant evaluation of food. Taken together, the work provides key insight into the spatial and temporal dynamics of the human insula-opercular network during food intake.

### REFERENCES IN THIS EXAMPLE

1. Saper, C. B., Chou, T. C. & Elmquist, J. K. The need to feed: Homeostatic and hedonic control of eating. Neuron vol. 36 199-211 (2002).
2. Lutter, M. & Nestler, E. J. Homeostatic and Hedonic Signals Interact in the Regulation of Food Intake. J. Nutr. 139, 629-632 (2009).
3. Morris, M. J., Beilharz, J. E., Maniam, J., Reichelt, A. C. & Westbrook, R. F. Why is obesity such a problem in the 21st century? The intersection of palatable food, cues and reward pathways, stress, and cognition. Neuroscience and Biobehavioral Reviews vol. 58 36-45 (2015).
4. Reiter, A. M. F., Heinze, H. J., Schlagenhauf, F. & Deserno, L. Impaired Flexible Reward-Based Decision-Making in Binge Eating Disorder: Evidence from Computational Modeling and Functional Neuroimaging. Neuropsychopharmacology 42, 628-637 (2017).
5. Livneh, Y. et al. Homeostatic circuits selectively gate food cue responses in insular cortex. Nature 546, 611-616 (2017).
6. Gehrlach, D. A. et al. Aversive state processing in the posterior insular cortex. Nat. Neurosci. 22, 1424-1437 (2019).
7. Schiff, H. C. et al. An insula-central amygdala circuit for guiding tastant-reinforced choice behavior. J. Neurosci. 38, 1418-1429 (2018).
8. Livneh, Y. et al. Estimation of Current and Future Physiological States in Insular Cortex. Neuron (2020) doi:10.1016/j.neuron.2019.12.027.
9. Spierling, S. et al. Insula to ventral striatal projections mediate compulsive eating produced by intermittent access to palatable food. Neuropsychopharmacology 45, 579-588 (2019).
10. Kusumoto-Yoshida, I., Liu, H., Chen, B. T., Fontanini, A. & Bonci, A. Central role for the insular cortex in mediating conditioned responses to anticipatory cues. Proc. Natl. Acad. Sci. U.S.A. 112, 1190-1195 (2015).
11. Stice, E., Spoor, S., Bohon, C., Veldhuizen, M. G. & Small, D. M. Relation of Reward From Food Intake and Anticipated Food Intake to Obesity: A Functional Magnetic Resonance Imaging Study. J. Abnorm. Psychol. 117, 924-935 (2008).
12. Chikazoe, J., Lee, D. H., Kriegeskorte, N. & Anderson, A. K. Distinct representations of basic taste qualities in human gustatory cortex. Nat. Commun. 10, (2019).
13. De Araujo, I. E. & Simon, S. A. The gustatory cortex and multisensory integration. Int. J. Obes. 33, S34-S43 (2009).
14. Stice, E., Spoor, S., Bohon, C. & Small, D. M. Relation between obesity and blunted striatal response to food is moderated by TaqIA A1 allele. Science 322, 449-52 (2008).
15. Stice, E. & Yokum, S. Gain in body fat is associated with increased striatal response to palatable food cues, whereas body fat stability is associated with decreased striatal response. J. Neurosci. 36, 6949-6956 (2016).
16. Babbs, R. K. et al. Decreased caudate response to milkshake is associated with higher body mass index and greater impulsivity. Physiol. Behav. 121, 103-111 (2013).
17. Bohon, C. & Stice, E. Negative affect and neural response to palatable food intake in bulimia nervosa. Appetite 58, 964-970 (2012).
18. Stice, E., Yokum, S., Blum, K. & Bohon, C. Weight gain is associated with reduced striatal response to palatable food. J. Neurosci. 30, 13105-13109 (2010).
19. Liu, S. & Parvizi, J. Cognitive refractory state caused by spontaneous epileptic high-frequency oscillations in the human brain. Sci. Transl. Med. 11, (2019).
20. Destrieux, C., Fischl, B., Dale, A. & Halgren, E. Automatic parcellation of human cortical gyri and sulci using standard anatomical nomenclature. Neuroimage 53, 1-15 (2010).
21. Kelly, C. et al. A convergent functional architecture of the insula emerges across imaging modalities. Neuroimage 61, 1129-1142 (2012).
22. Oostenveld, R., Fries, P., Maris, E. & Schoffelen, J. M. FieldTrip: Open source software for advanced analysis of MEG, EEG, and invasive electrophysiological data. Comput. Intell. Neurosci. 2011, (2011).
23. Li, G. et al. Optimal referencing for stereo-electroencephalographic (SEEG) recordings. Neuroimage 183, 327-335 (2018).
24. Maris, E. & Oostenveld, R. Nonparametric statistical testing of EEG- and MEG-data. J. Neurosci. Methods 164, 177-190 (2007).
25. Foster, B. L., Rangarajan, V., Shirer, W. R. & Parvizi, J. Intrinsic and task-dependent coupling of neuronal population activity in human parietal cortex. Neuron 86, 578-590 (2015).
26. Dastjerdi, M., Ozker, M., Foster, B. L., Rangarajan, V. & Parvizi, J. Numerical processing in the human parietal cortex during experimental and natural conditions. Nat. Commun. 4, 1-11 (2013).
27. Levitan, D. et al. Single and population coding of taste in the gustatory cortex of awake mice. J. Neurophysiol. 122, 1342-1356 (2019).
28. Hollander, J. A., Lu, Q., Cameron, M. D., Kamenecka, T. M. & Kenny, P. J. Insular hypocretin transmission regulates nicotine reward. Proc. Natl. Acad. Sci. U.S.A. 105, 19480-19485 (2008).
29. Contreras, M., Ceric, F. & Torrealba, F. Inactivation of the interoceptive insula disrupts drug craving and malaise induced by lithium. Science (80-.). 318, 655-658 (2007).
30. Balleine, B. W. & Dickinson, A. The effect of lesions of the insular cortex on instrumental conditioning: Evidence for a role in incentive memory. J. Neurosci. 20, 8954-8964 (2000).
31. Gardner, M. P. H. & Fontanini, A. Encoding and tracking of outcome-specific expectancy in the gustatory cortex of alert rats. J. Neurosci. 34, 13000-17 (2014).
32. Samuelsen, C. L., Gardner, M. P. H. & Fontanini, A. Effects of Cue-Triggered Expectation on Cortical Processing of Taste. Neuron 74, 410-422 (2012).
33. Wu, H. et al. Closing the loop on impulsivity via nucleus accumbens delta-band activity in mice and man. Proc. Natl. Acad. Sci. U.S.A. 115, 192-197 (2018).
34. Crouzet, S. M., Busch, N. A. & Ohla, K. Taste quality decoding parallels taste sensations. Curr. Biol. 25, 890-896 (2015).
35. Avery, J. A. et al. Taste quality representation in the human brain. J. Neurosci. 1751-19 (2019) doi:10.1523/JNEUROSCI.1751-19.2019.
36. Gehrlach, D. A. et al. Aversive state processing in the posterior insular cortex. doi:10.1038/s41593-019-0469-1.
37. Duerden, E. G., Arsalidou, M., Lee, M. & Taylor, M. J. Lateralization of affective processing in the insula. Neuroimage 78, 159-175 (2013).
38. Simmons, W. K. et al. Category-specific integration of homeostatic signals in caudal but not rostral human insula. Nat. Neurosci. 16, 1551-1552 (2013).
39. Ogawa, H. et al. Functional MRI detection of activation in the primary gustatory cortices in humans. Chem. Senses 30, 583-592 (2005).
40. Faurion, A. et al. Human taste cortical areas studied with functional magnetic resonance imaging: Evidence of functional lateralization related to handedness. Neurosci. Lett. 277, 189-192 (1999).
41. Benjamin, R. M. & Burton, H. Projection of taste nerve afferents to anterior opercular-insular cortex in squirrel monkey (Saimiri sciureus). Brain Res. 7, 221-231 (1968).
42. Witt, M. Anatomy and development of the human taste system. in Handbook of Clinical Neurology vol. 164 147-171 (Elsevier B.V., 2019).
43. Mak, Y. E., Simmons, K. B., Gitelman, D. R. & Small, D. M. Taste and olfactory intensity perception changes following left insular stroke. Behav. Neurosci. 119, 1693-1700 (2005).
44. Huerta, C. I., Sarkar, P. R., Duong, T. Q., Laird, A. R. & Fox, P. T. Neural bases of food perception: Coordinate-based meta-analyses of neuroimaging studies in multiple modalities. Obesity 22, 1439-1446 (2014).
45. Ghaziri, J. et al. Subcortical structural connectivity of insular subregions. Sci. Rep. 8, (2018).

### Example 4: Human Bi-Accumbens Network Analysis Dissociates Naturalistic Behavioral States

Introduction: A current first-in-human clinical trial is underway using deep responsive neurostimulation (RNS) of the bilateral Nucleus Accumbens (NAc) as an emerging therapy for loss of control (LOC) eating. Ambulatory connectivity and graph architecture of the bilateral human NAc network has never been explored, and yet could greatly improve conventional signals (i.e. low-frequency spectral power) which could be limited in their resolution and specificity for behavioral states. Here, data from two initial subjects implanted with NAc RNS system investigating connectivity and architecture within the bilateral ventral and dorsal NAc as it relates to naturalistic behavioral states was investigated.

Objective: Explore connectivity and architecture patterns of the bilateral ventral and dorsal NAc that can distinguish behavioral states.

Methods: Data were obtained from two participants with binge-eating disorder who were surgically implanted with bilateral NAc RNS (NeuroPace, Inc) leads. Ambulatory recordings were acquired and time-locked using the magnet-swipe feature of the RNS system. Electrographic recordings were triggered by the patient in ambulatory periods of cravings with hunger, cravings without hunger, random sleep, and random awake states. Phase-Locking Value (PLV)-based connectivity and communicability (Qexp)-based graph architecture were compared during the 60 seconds preceding these behavioral states. Specific analyses tested whether these metrics could differentiate cravings without hunger from two key behavioral states—cravings with hunger and the sleep state—which share behavioral and spectral features with cravings without hunger, respectively.

Results: Bilateral ventral NAc connectivity was elevated specific to episodes of craving in the absence of hunger. Similarly, bilateral network communicability was specifically elevated during cravings without hunger compared to states without craving. Further, ventral bilateral NAc connectivity was elevated in sleep compared to awake states, but was significantly reduced compared to cravings without hunger.

Conclusion: This is the first analysis of bilateral network activity during ambulatory, naturalistic behavior in humans. Evidence for a role of ventral bilateral NAc connectivity and graph architecture in differentiating craving vs. hunger states was observed. Moreover, connectivity differentiated craving from sleep, which is unique to this method given shared predominance of low frequency rhythms in these states. This analytical framework could inform how NAc connectivity/architecture patterns mediate various behavioral states and refine the control signal for NAc RNS in LOC eating, and closed-loop approaches in general.

TABLE 2
Participant characteristics

| | | | | | | | Preferred | | | |
|---|---|---|---|---|---|---|---|---|---|---|
| | | | | | | Palatable | Palatable | Number of Contacts | | |
| | | | Handed- | | | Liquid | over | Frontal | Ant. | Pos. |
| ID | Age | Sex | ness | Seizure Focus | BMI | Rating | Neutral? | Operculum | Insula | Insula |
| S1 | 59 | M | R | Bitemporal | 26 | 5/10 | Yes | 2(L) 2(R) | 6(L) 5(R) | 1(L) 2(R) |
| S2 | 34 | F | R | L. Lateral | 32 | 3/10 | Yes | 0 | 9(L) | 0 |
| | | | | Temporal | | | | | | |
| S3 | 24 | M | R | R. Mesial | 51 | 3/10 | Yes | 0 | 9(R) | 6(R) |
| | | | | Temporal | | | | | | |
| S4 | 49 | M | R | L. Frontal and | 31 | 8/10 | Yes | 7(L) | 1(L) | 7(L) |
| | | | | L. Mes. | | | | | | |
| | | | | Temporal | | | | | | |
| S5 | 51 | M | R | L. Mesial | 27 | 10/10 | Yes | 2(L) | 9(L) 5(R) | 4(L) |
| | | | | Temporal | | | | | | |
| S6 | 20 | M | R | R. Parietal/ | 19 | 5/10 | Yes | 0 | 9(R) | 0 |
| | | | | Occipital | | | | | | |
| S7 | 27 | M | R | R. Parietal | 24 | 8/10 | Yes | 1(L) | 7(L) | 3(L) |
| S8 | 36 | M | R | Bitemporal | 23 | 7/10 | Yes | 2(L) | 7(L) | 6(L) |
| S9 | 46 | M | R | R Mesial | 28 | 5/10 | Yes | 4(R) | 3(R) | 0 |
| | | | | Frontal | | | | | | |
| S10 | 29 | F | R | R Frontal | 41 | 3/10 | Yes | 2(L) 1(R) | 8(L) 4(R) | 6(R) |
| | | | | Dysplasia | | | | | | |
| S11 | 62 | M | R | No sz, pain | 29 | 10/10 | Yes | 3(L) | 10(L) 6(R) | 6(L) 3(R) |
| | | | | mapping | | | | | | |

TABLE 3
Correlation between task responses and subject attributes

| | | | |
|---|---|---|---|
| Type of Correlation | N (subjects) | R | P |

Cue-Responses

| | | | |
|---|---|---|---|
| % Responsive - BMI | 11 | −0.06 | 0.87 |
| % Specific - BMI | 11 | −0.38 | 0.24 |
| % Responsive - MS rating | 11 | 0.46 | 0.16 |
| % Specific - MS rating | 11 | 0.58 | 0.06 |
| % Responsive - Age | 11 | 0.21 | 0.54 |
| % Specific - Age | 11 | 0.10 | 0.77 |

Receipt- Responses

| | | | |
|---|---|---|---|
| % Responsive - BMI | 11 | 0.06 | 0.86 |
| % Specific - BMI | 11 | −0.37 | 0.29 |
| % Responsive - MS rating | 11 | 0.18 | 0.60 |
| % Specific - MS rating | 11 | −0.03 | 0.93 |
| % Responsive - Age | 11 | 0.09 | 0.79 |
| % Specific - Age | 11 | −0.28 | 0.44 |

TABLE 4
MNI coordinates of cue and/or receipt-specific responses

| # | Contact Location | Response | x | y | z |
|---|---|---|---|---|---|
| 1 | L Anterior Circular Sulcus | Cue-Specific | −27.72 | 17.69 | −3.39 |
| | | Receipt-Specific | | | |
| 2 | R Superior Circular Sulcus | Cue-Specific | 33.67 | 15.36 | 13.84 |
| 3 | L Short Insular Gyri | Cue-Specific | −40.19 | 14.94 | −5.21 |
| 4 | L Frontal Operculum | Cue-Specific | −44.86 | 26.26 | 19.93 |
| | | Receipt-Specific | | | |
| 5 | L Long Gyrus and Central Sulcus | Cue-Specific | −37.87 | −6.78 | 1.83 |
| 6 | L Long Gyrus and Central Sulcus | Cue-Specific | −37.02 | −5.16 | 4.87 |
| 7 | L Long Gyrus and Central Sulcus | Cue-Specific | −36.16 | −3.54 | 7.92 |
| | | Receipt-Specific | | | |
| 8 | L Short Insular Gyri | Cue-Specific | −34.28 | 13.10 | −4.16 |
| 9 | L Superior Circular Sulcus | Cue-Specific | −27.02 | 21.73 | 9.12 |
| 10 | L Long Gyrus and Central Sulcus | Cue-Specific | −38.93 | −10.27 | 10.54 |
| 11 | L Long Gyrus and Central Sulcus | Cue-Specific | −39.07 | −13.73 | 13.22 |
| 12 | L Long Gyrus and Central Sulcus | Cue-Specific | −39.20 | −17.19 | 15.91 |
| 13 | L Frontal Operculum | Cue-Specific | −32.97 | 25.96 | 7.28 |
| 14 | L Inferior Circular Sulcus | Cue-Specific | −42.12 | −8.95 | −5.99 |
| 15 | L Inferior Circular Sulcus | Cue-Specific | −41.01 | −9.09 | −0.50 |
| 16 | L Long Gyrus and Central Sulcus | Cue-Specific | −40.07 | −9.16 | 5.086 |
| 17 | L Anterior Circular Sulcus | Cue-Specific | −30.59 | 15.90 | 12.35 |
| 18 | L Short Insular Gyri | Cue-Specific | −36.47 | 9.16 | −11.71 |
| 19 | L Short Insular Gyri | Cue-Specific | −35.83 | 10.91 | −9.51 |
| 20 | R Long Gyrus and Central Sulcus | Receipt-Specific | 40.60 | 3.52 | −8.37 |
| 21 | L Superior Circular Sulcus | Receipt-Specific | −31.68 | 20.54 | 13.72 |
| 22 | L Frontal Operculum | Receipt-Specific | −38.26 | 20.02 | 6.57 |
| 23 | L Superior Circular Sulcus | Receipt-Specific | −28.81 | 19.51 | 5.81 |
| 24 | L Long Gyrus and Central Sulcus | Receipt-Specific | −36.38 | −8.65 | 6.45 |
| 25 | R Superior Circular Sulcus | Receipt-Specific | 36.55 | 13.86 | −4.22 |
| 26 | R Superior Circular Sulcus | Receipt-Specific | 35.26 | 14.58 | −0.16 |
| 27 | R Short Insular Gyri | Receipt-Specific | 41.69 | 4.69 | 2.59 |
| 28 | R Superior Circular Sulcus | Receipt-Specific | 34.51 | −8.13 | 10.80 |
| 29 | R Inferior Circular Sulcus | Receipt-Specific | 38.01 | −21.08 | 4.72 |

1. A method of detecting a low frequency modulation in the insular cortex and the hippocampus of a subject, wherein said subject is diagnosed with, or suspected of having, an impaired inhibitory control disorder (ICD), the method comprising:
inserting at least one electrode into each the insular cortex and the hippocampus of the subject; and
recording brain wave activity in the insular cortex and the hippocampus of the subject.

2. The method of claim 1, wherein said low frequency modulation comprises a modulation having a frequency between about 0 hertz-8 hertz.

3. The method of claim 1, wherein said low frequency modulation comprises a modulation having a frequency between about 3.5 to 7.5 Hz.

4. The method of claim 1, wherein said low frequency modulation comprises a modulation having a frequency between about 0.1-4 Hz.

5. The method of claim 1, wherein said low frequency modulation is an increase of a frequency between about 0 hertz-8 hertz relative to a standard control.

6. The method of claim 1, wherein the ICD comprises a disorder that is associated with a lack of impulse control, and wherein the ICD includes one or more of binge eating, substance abuse, sex addiction or compulsive sexuality, kleptomania, pyromania, trichotillomania, panic disorder, Intermittent Explosive Disorder, compulsive behaviors including gambling, night eating, loss of control eating, emotional or stress eating, compulsive eating, purge behaviors, or suicidal ideation/attempt.

7. The method of claim 1, further comprising administering, in response to the low frequency modulation, a stimulation to the insular cortex and the hippocampus of the subject.

8. The method of claim 7, wherein said stimulation is transcranial direct current stimulation (TDCS), transcranial magnetic stimulation (TMS), or low intensity ultrasound stimulation.
